(19) United States
(12) Patent Application Publication
(10) Pub. No.: US 2005/0009743 A1
(43) Pub. Date: Jan. 13, 2005

(54) In vitro assays for inhibitors of hiv capsid conformational changes and for hiv capsid formation

(76) Inventors: Wesley I Sundquist, Salt Lake City, UT (US); Hui Wang, Salt Lake City, UT (US); Christopher P Hill, Salt Lake City, UT (US); Timothy L Stemmler, Livonia, MI (US); Darrell R Davis, Salt Lake City, UT (US); Steve Alam, Midvale, UT (US)

Correspondence Address: NEEDLE & ROSENBERG, P.C., SUITE 1000, 999 PEACHTREE STREET, ATLANTA, GA, 30309-3915, US

(21) Appl. No.: 10/484,784

(22) PCT Filed: Jul. 26, 2002
(86) PCT No.: PCT/US02/23875

Related U.S. Application Data
(60) Provisional application No. 60/307,998, filed on Jul. 26, 2001.

(51) Int. Cl. A61K 38/17; G01N 33/53; C07K 14/47

(52) U.S. Cl. 514/12; 435/7.1; 530/409

(57) ABSTRACT

Disclosed are methods and compositions for assays related to particle formation of the HIV virus.

### I. CROSS REFERENCE TO RELATED APPLICATIONS

This application claims benefit of U.S. Provisional Application No. 60/333,553, filed Nov. 26, 2001, and U.S. Provisional Application No. 60/307,998, filed Jul. 26, 2001, both of which are hereby incorporated herein by reference in their entirety.

### II. ACKNOWLEDGMENTS

This invention was made with government support under Grants NIH RO1 AI45405 and AI43036. The government has certain rights in the invention.

### III. BACKGROUND OF THE INVENTION

Disclosed are methods and compositions which can be used in high-throughput screening (HTS) for inhibitors of conformational changes that accompany HIV capsid maturation, inhibitors of CA protein dimerization, and for inhibitors of HIV-1 assembly.

Viruses must be packaged and processed before they become infective. The packaging and processing process for viruses, such as HIV-1, involves many steps. For example, HIV-1 packaging involves formation of a particle by assembly of approximately 4000 copies of the HIV Gag protein. This Gag protein is then proteolytically processed to produce a number of other proteins and peptides, including CA, or capsid protein. In addition to many other activities, the CA protein must go through a maturation step which involves structural rearrangements.

The disclosed methods and compositions allow for the identification of inhibitors of the various processing maturation events that must take place for infectious viral production.

### IV. SUMMARY OF THE INVENTION

In accordance with the purposes of this invention, as embodied and broadly described herein, this invention, in one aspect, relates to compositions and methods for in vitro maturation assays and compositions and methods that inhibit capsid maturation and in another aspect relates to compositions and methods for in vitro assembly assays.

Additional advantages of the invention will be set forth in part in the description which follows or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### V. BRIEF DESCRIPTION OF THE DRAWINGS

The accompanying drawings, which are incorporated in and constitute a part of this specification together with the description serve to explain the principles of the invention.

FIG. 1A shows schematic illustrations of the immature and mature HIV-1 virions. Structures formed by the CA polypeptide are highlighted, with the N- and C-terminal domains represented as hollow squares and spheres, respectively. Locations of the viral RNA, envelope proteins (SU and TM), and other Gag-derived polypeptides (MA and NC) are also shown. FIG. 1B shows the domain organization of HIV-1 Gag, and locations of the 5 viral protease cleavage sites (vertical lines). Amino acid numbering schemes for HIV-1_{NL4-3} Gag and the MA-CA protein constructs as disclosed herein except where noted or obviously using a different scheme are shown. FIG. 1C shows a comparison of ¹⁵N filtered HSQC spectra for deltaMA-CA_{278, 129}MA-CA₂₇₈, and CA₂₈₃, superimposed on each other. FIG. 1D shows a schematic representation of the proteolytic processing of HIV-1 Gag. The ordered processing of HIV-1 Gag and the relative rates of proteolysis at the different processing sites are depicted.

FIG. 2 shows structures of ₁₂₉MA-CA₂₇₈ and CA₂₇₈ FIG. 2A shows the primary sequence, secondary structures, and coding for ₁₂₉MA-CA₂₇₈ (I) and CA₂₇₈ (II). FIG. 2B shows the stereoview of the best-fit superposition of the backbone atoms of the 20 lowest penalty ₁₂₉MA-CA₂₇₈ structures. FIG. 2C shows a ribbon diagram of the ₁₂₉MA-CA₂₇₈ structure.

FIG. 3 shows β-hairpin “Switch” of HIV-1 CA. FIG. 3A shows packing interactions between the N-terminal β-hairpin and helices 1 and 3 that stabilize the hairpin down conformation of ₁₂₉MA-CA₂₇₈. This interface is well defined by a total of 65 long range NOE's between the hairpin and adjacent helices. Apparent H-bonding or salt bridges (dashed lines) and van der Waals contacts between the hairpin and helices I and III (arrows) are shown. Note that additional long range contacts between strand 1 and helix 6 are not shown. FIG. 3B shows a superposition of the P-hairpin regions of ₁₂₉MA-CA₂₇₈ (darker) and CA₂₇₈ (lighter). Xxx need to find this figure. FIG. 3C shows a summary of the structural changes that convert ₁₂₉MA-CA₂₇₈ into CA₂₇₈ upon viral protein proteolytic cleavage at the MA-CA junction of Gag (scissors). Changes include: inversion of the N-terminal CA β-hairpin (curved arrow), unfolding of the type II turn, replacement of the Asp 183 . . . His 144 salt bridge with the Pro133-Asp183 salt bridge (dashed lines), and shifting of the register between helices 1 and 2 by one helical repeat (green arrow). This shift positions these two helices to oligomerize into a 12 helical bundle in the mature CA hexamer (represented by red arrows)⁴⁰.

FIG. 4 shows the pH dependence of CA structure and assembly. FIG. 4A shows a His Nε2 nitrogen chemical shifts in ₁₂₉MA-CA₂₇₈ as a function of pH. Chemical shift changes for the five histidine Nε2 nitrogens (H144(♦), H195(◯), H217(▴), H219 (▪), and H252(●)) are displayed for pH values (uncorrected for 90% H₂O, 26 C) ranging from 5.25 to 7.8. Hε1, Nε, Hδ2 and Nδ1 shifts were taken from a series of long-range HSQC spectra collected at ten pH values between 5.3 and 7.9. FIG. 413 shows higher order CA assemblies formed at pH 6.0 (left panel) and 8.0 (right panel). Assembly conditions are given in the text.

FIG. 5 shows conformational states of the N-terminal domain of HIV-1 CA. FIG. 5A shows a summary of the different conformations of the CA N-terminal domain and the conditions that favor their formation. FIG. 5B shows a space filling model showing a potential inhibitor binding pocket in the hairpin down conformation of HIV-1 CA. Net electrostatic charges are coded, and the Asp183 side chain is shown explicitly.

FIG. 6 shows the different accessibility of Ile247 in the immature (₁₂₉MA-CA₂₇₈) and mature (CA₁₃₃₋₂₇₈) CA structures. The side chain of Ile247 is set forth. The first strand of the β-hairpin is translucent in the mature structure.

FIG. 7A shows Protein expression and purification by SDS-PAGE analysis of the expression and purification of ₁₀₅MA-CA₂₇₈(His)₆ protein. Lane 1, molecular weight standards; lane 2, total cellular BL21(DE3) E.coli proteins prior to induced expression of the ₁₀₅MA-CA₂₇₈(His)₆ protein; lane 3, total cellular BL21(DE3) E.coli proteins following induction of the ₁₀₅MA-CA₂₇₈(His)₆ protein; lane 4, purified ₁₀₅MA-CA₂₇₈(His)₆ protein. FIG. 7B shows the SDS-PAGE analysis of the expression and purification of CA₁₃₃₋₂₇₈(His)₆ protein. Lane 1, molecular weight standards; lane 2, total cellular BL21(DE3) E.coli proteins prior to induced expression of the CA₁₃₃₋₂₇₈(His)₆ protein; lane 3, total cellular BL21 (DE3) E. coli proteins following induction of the CA₁₃₃₋₂₇₈(His)₆ protein; lane 4, purified CA₁₃₃₋₂₇₈(HiS)6 protein.

FIG. 8. Chemical reactivity of Cys247 in ₁₀₅MA-CA₂₇₈(His)₆ and CA₁₃₃₋₂₇₈(HiS)₆. The proteins were mixed in equimolar concentrations, labeled with [³H] N-Ethylnaleimide (NEM), and separated by SDS-PAGE. A) The protein mixture was detected by Coomassie blue staining and quantitated. B) The protein mixture was detected by fluorography and quantitated.

FIG. 9 shows the expression and purification of HIV-1 CA-NC(G94D). Lane 1, molecular weight standards; lane 2, total cellular BL21(DE3) E.coli proteins prior to induced expression of the CA-NC(G94D) protein; lane 3, total cellular BL21(DE3) E.coli proteins following induction of the CA-NC(G94D) protein; lane 4, purified CA-NC(G94D) protein.

FIG. 10 shows negatively stained TEM image of HV-1 CA-NC(G94D)/d(TG)₅₀ assembled in vitro. (A) Low magnification (3000×), bar=2 microns. (B) Higher magnification (30000×), bar=500 mn.

FIG. 11 shows protein expression and purification of (CA-CTD)₂ protein. A) SDS-PAGE analysis of the expression and purification of (CA-CTD)₂ protein. Lane 1, molecular weight standards; lane 2, total cellular BL21 (DE3) E. coli proteins prior to induced expression of the (CA-CTD)₂ protein; lane 3, total cellular BL21(DE3) E.coli proteins following induction of the (CA-CTD)₂ protein; lane 4, purified (CA-CTD)₂ protein. B) SDS-PAGE analysis of the expression and purification of (CA-CTD)₂-FLAG protein. Lane 1, molecular weight standards; lane 2, total cellular BL21(DE3) E.coli proteins prior to induced expression of the (CA-CTD)₂-FLAG protein; lane 3, total cellular BL21(DE3) E.coli proteins following induction of the (CA-CTD)₂-FLAG protein; lane 4, purified (CA-CTD)₂-FLAG protein.

FIG. 12 shows dimerization of CA(CA-CTD)₂, tested by A) Superdex 75 gel filtration chromatograph of (CA-CTD). B) Equilibrium sedimentation profile and fit residuals for (CA-CTD).

FIG. 13 shows negative-stain EM images of HIV-1 CA-NC (G94D)/d(TG)so assembled in vitro. Magnification (10000×), bar=500 nm (A) CA-NC (CA G94D). (B) CA-NC (wild-type).

FIG. 14 shows negative-stain EM images of HIV-1 CA-NC/Oligonucleotide assembled in vitro. Magnification (10000×), bar=500 mn (A) d(TG)₂₅. (B) d(TG)₃₈. (C) d(TG)₅₀. (D) d(N)₁₀₀. At higher concentrations assembly will occur with random oligonucleotides, for example, above 100 uM).

FIG. 15 shows negative-stain EM images of HIV-1 CA-NC/d(TG)₅₀ assemblies. Magnification (10000×), bar=500 nm (A) CA-NC(G94D) (control). (B) CA(A42D) mutant (C) CA(W184A/M185A) mutant.

### VI. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific compositions , or to particular formulations, as such may, of course, vary. It is also to be understood that the terminology used herein is for the purpose of describing particular embodiments only and is not intended to be limiting.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon. Furthermore, references may be cited along with a letter, such as (3B). This letter refers to particular reference list disclosed herein, designated with the letter. Furthermore, should a letter not be associated with a reference number, it will be clear to the slilled artisan, from the context and the potential references, which reference is being relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular CA protein is disclosed and discussed and a number of modifications that can be made to a number of molecules including CA proteins are discussed, specifically contemplated is each and every combination and permutation of CA protein and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus means for example, that combinations A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

A. Compositions and Methods

1. Viral Processing

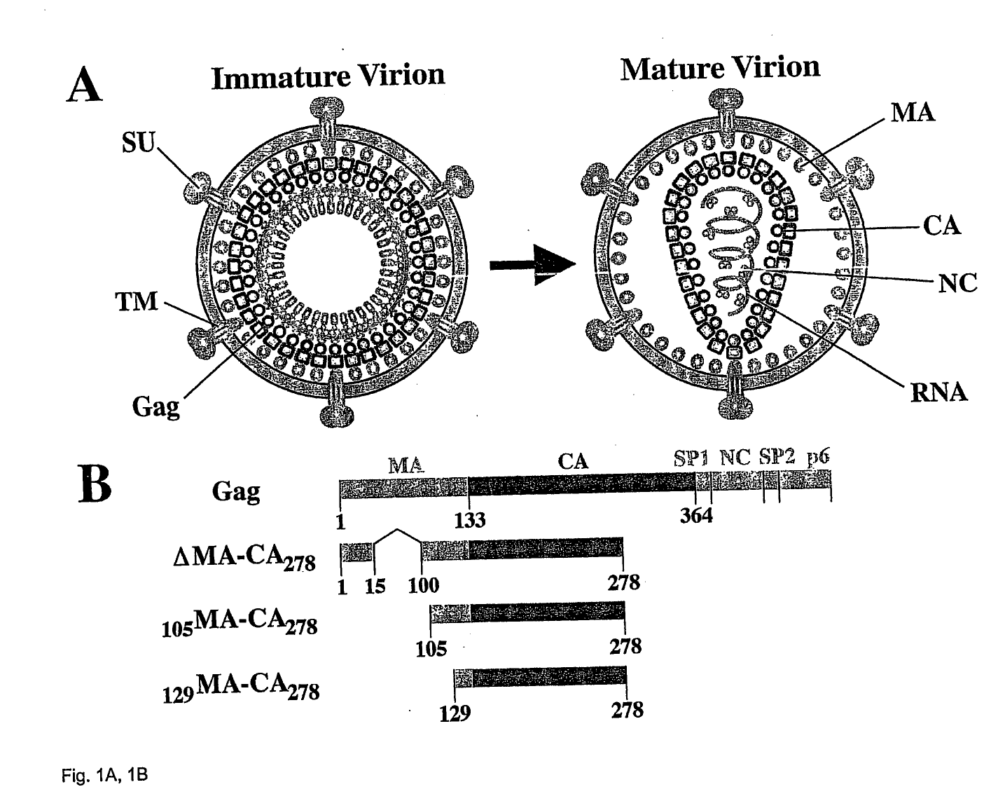

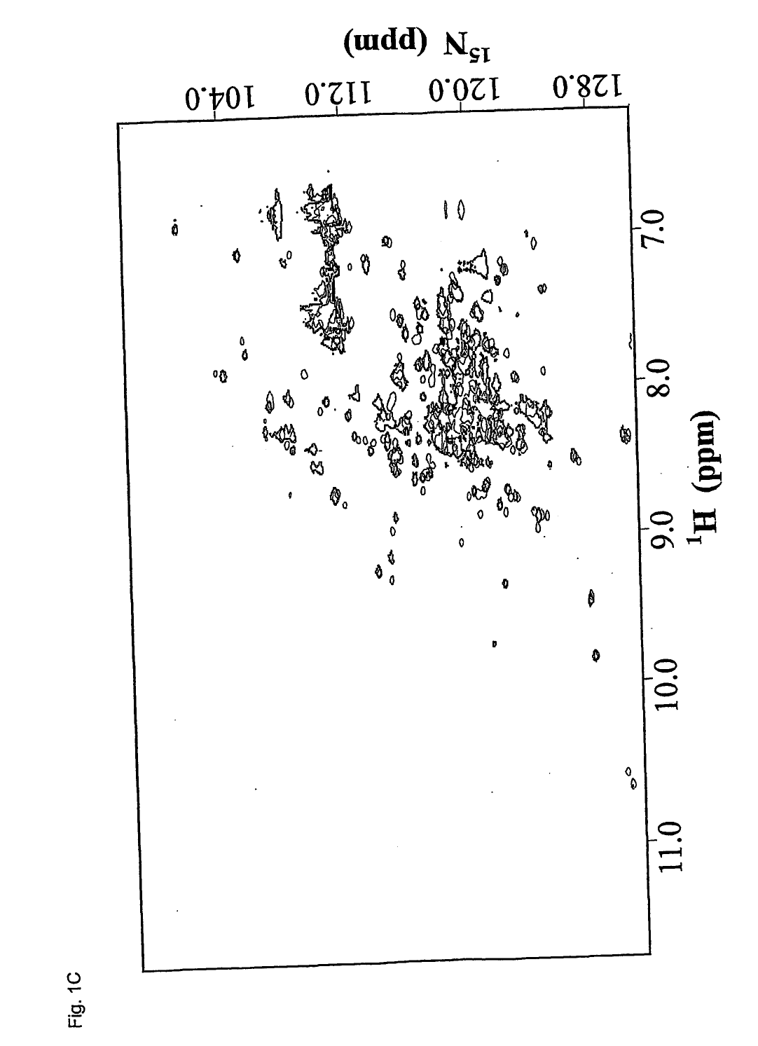

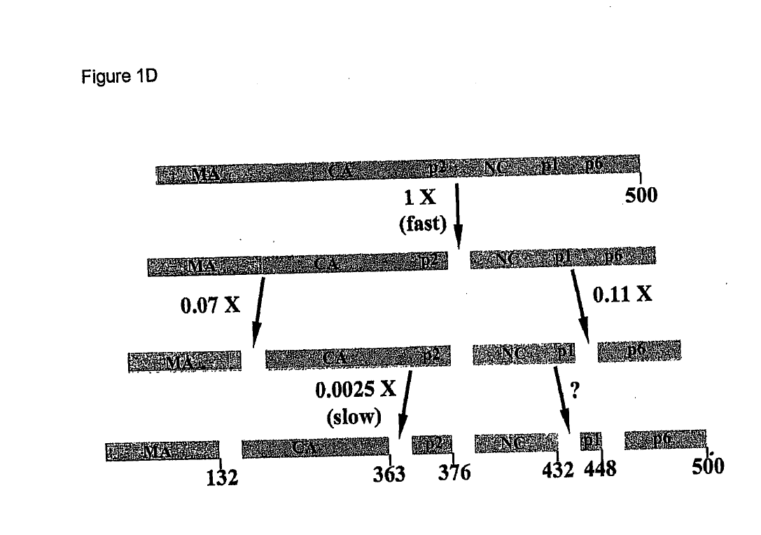

The human immunodeficiency virus type 1 (HIV-1) initially assembles as an immature viral particle, containing a spherical shell composed of Gag polyproteins underneath the viral inner membrane. Before HIV can become an infectious particle, the coat proteins and nucleic acids must be assembled together. This assembly begins by the polymerization of the Gag polyprotein (approximately 4000 copies) (FIG. 1). Concomitant with budding, the Gag protein is proteolytically processed at five sites to form three distinct structural proteins. These proteins are, starting at the NH₂ terminal end of Gag, matrix protein (MA) which binds to the membrane, capsid (CA), which directs major protein contacts necessary for assembly, and the COOH terminal end of Gag contains the nucleocapsid protein (NC) which packages the RNA genome. Also produced are three small peptides: p2, p1, and p6 after these cleavage events (reviewed by Krausslich, 1996) (or SP2 and SP1)(1). Maturation of the HIV-1 virion involves a series of complex transformations, including: 1) rearrangement of the dimeric RNA genome into a more stable conformation(2), 2) condensation of the NC/RNA complex (and its associated nucleic acid processing enzymes) into a dense central mass, and 3) reassembly of the processed CA protein into a conical shell (the “capsid”) that surrounds the RNA/NC complex(3). The process of viral maturation thus creates a new large (˜100 MDa) ribonucleoprotein complex that organizes the genome for uncoating and replication in a new host cell.

The HIV-1 Gag processing is temporally controlled, and the rates of cleavage at the different Gag sites differ dramatically both in vivo and in vitro (Erickson-Viitanen et al., 1989; Konvalinka et al., 1995; Krausslich et al., 1988; Tritch et al., 1991) (FIG. 1D). The initial cleavage of Gag occurs at the p2-NC junction, forming MA-CA-p2 and NC-p1-p6 intermediates. Gag is then cleaved at an approximately 10-fold slower rate at the MA-CA and p1-p6 junctions (FIG. 1). The final cleavage occurs at the CA/p2 junction, at a 400-fold slower rate. The sequential processing of the Gag polyprotein, particularly at the N- and C-terminus of CA, is important for particle maturation and viral infectivity, as mutations that block the cleavage at either end of CA result in the formation of noninfectious particles with distinctly abnormal morphologies (Gottlinger et al., 1989; Pettit et al., 1994; Wiegers et al., 1998). Specifically, these mutations prevent the condensation of CA core, and instead result in a thin electron-dense layer near the viral membrane. Therefore, it is believed that proteolytic liberation of both the N- and C-terminus of CA triggers capsid rearrangements by altering the structure of CA. Two molecular switches may function during the maturation transition: 1) cleavage at MA-CA junction frees the N-terminus of CA to initiate condensation of the conical core, and 2) cleavage at CA-p2 somehow frees the C-terminal of CA, to allow core assembly to proceed to completion (Gross et al., 2000; Wiegers et al., 1998).

Following proteolysis, the virion undergoes morphological changes (maturation), characterized by the condensation of CA protein into a conical core encasingNC and RNA genome of HIV-1. CA dissociating from the spherical shell to form the central conical capsid is the hallmark of the mature, infectious virus. (for a review of Gag see H-G Krausslich Ed. Morphogenesis and Maturation of Retroviruses Vol 214 Current Trends in Microbiology and Immunology (Springer-Verlag, Berlin 1996) and Swanstrom R. And Willis J. W. in Retroviruses J. M. Coffin S. H. Hughes, and H. E. Varmus Eds. (Cold Spring harbor Laboratory press Plainview N.Y., 1997 pp 263-334) both of which are herein incorporated by reference for at least the discussions of the Gag polypeptide.). The Gag polypeptide is processed by the viral protease which cleaves the polypeptide into the three discreet proteins (and three smaller peptides) which then interact to form the infectious viral particle.

Virus assembly often involves a maturation step, where the procapsid of the immature virion undergoes a large-scale, irreversible conformational change to form the capsid of the mature virion. Such maturation transitions have been characterized for many viruses, including dsRNA phages, insect viruses, and herpesviruses as well as retroviruses (Butcher et al., 1997; Canady et al., 2000; Trus et al., 1996; Turner and Summers, 1999). These transitions are triggered by various signals, including DNA packaging, receptor binding, and proteolytic processing of the coat protein (Chow et al., 1997; Duda et al., 1995; von Schwedler et al., 1998). Electron microscopy and image reconstruction analyses have revealed that maturation usually involves dramatic structural rearrangements of the coat proteins, and the coat proteins can adopt different conformations and intersubunit interactions in procapsid and capsid structures. For example, in bacteriophage HK97, capsid maturation involves large subunit rotations and local refolding (Conway et al., 2001). These studies have also revealed that even in the static structure of fully mature viral capsids, individual protein subunits often adopt different structures that allow the capsid to form a closed structure of defined morphology.

Recombinant CA proteins exhibit similar structural polymorphism in vitro, with long helical tubes favored at high pH, short tubes and cones favored at low pH, and spheres favored by CA proteins with N-terminal MA extensions.

Three dimensional structures of fully processed HIV-1 MA, CA, and NC proteins have been determined, and these structures presumably represent mature protein conformations (reviewed in 4). The 14 kDa MA protein is composed of an N-myristolyated membrane targeting segment, a globular central domain (residues 7-105) and a disordered C-terminal tail (105-132). This domain directs Gag to assembly sites on the plasma membrane (5-15) and helps recruit the viral envelope protein onto the virion surface(16-21),but does not appear to play a critical structural role, as Gag mutants that are missing the MA domain can still assemble and bud from cells, and are even infectious under some conditions (22).

In vitro assembly systems using recombinant Gag proteins have been utilized to study the structures of immature and mature HIV-1 particles. CA and CA-p2-NC proteins form cylinders and cones (Campbell and Vogt, 1995; Ganser et al., 1999; Gross et al., 1997; Li et al., 2000), that resemble the mature capsid, while constructs in which the N-terminus of CA is extended (by as few as four MA residues) assemble into spheres (Campbell et al., 2001; Campbell and Rein, 1999; Gross et al., 1998; Gross et al., 2000; von Schwedler et al., 1998), that apparently mimick the immature virion. In addition, deletion of the p2 peptide in the context of MA-CA-NC proteins can revert spherical assemblies to cylinders. These results further indicate that cleavage sites at either end of CA act as conformational switches to determine the morphology of HV-1 particles.

The dramatic morphological changes that accompany HIV-1 assembly and maturation imply that the structures and protein-protein interactions of the different Gag subunits must also change as the virus matures. Each Gag cleavage event is essential for viral replication, and blockage of the different cleavage sites arrests maturation at morphologically distinct stages, suggesting that Gag processing proceeds through a temporally defined pathway in which the five Gag cleavage events facilitate distinct steps in viral maturation (45). Assembly studies indicate that the proteolytic cleavage sites at either end of CA function as structural “switches” that alter the equilibrium between mature and immature CA conformations. Thus, the removal of either N-terminal MA (36,38) or C-terminal SP1 extensions (14, 37) (e.g., by proteolysis) tends to formation of “mature” CA assemblies.

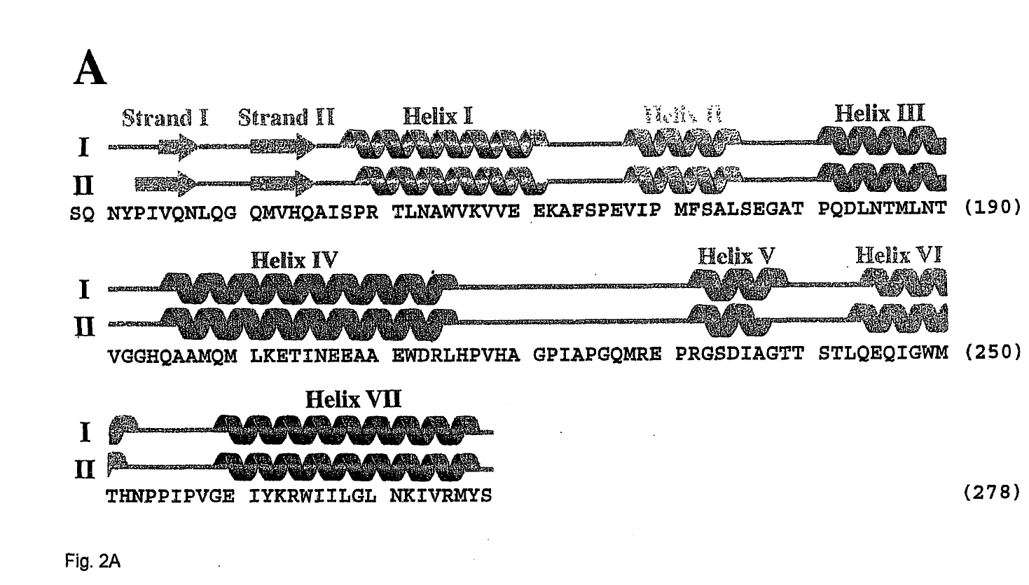

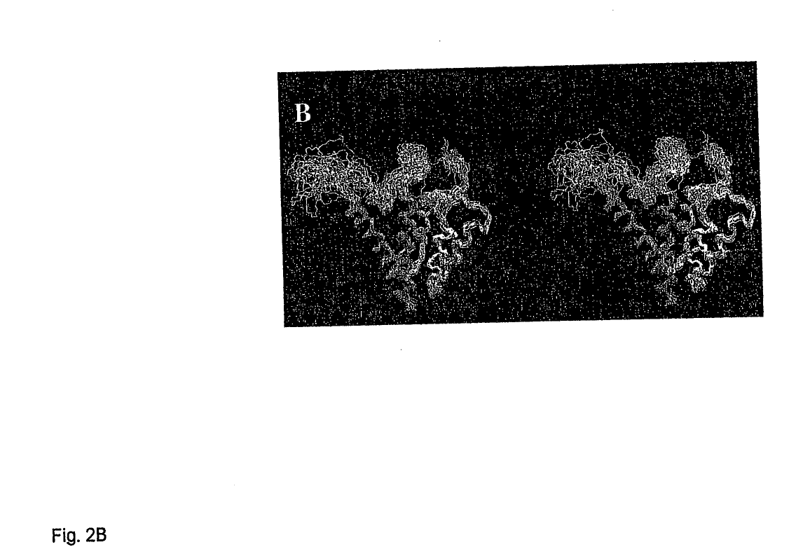

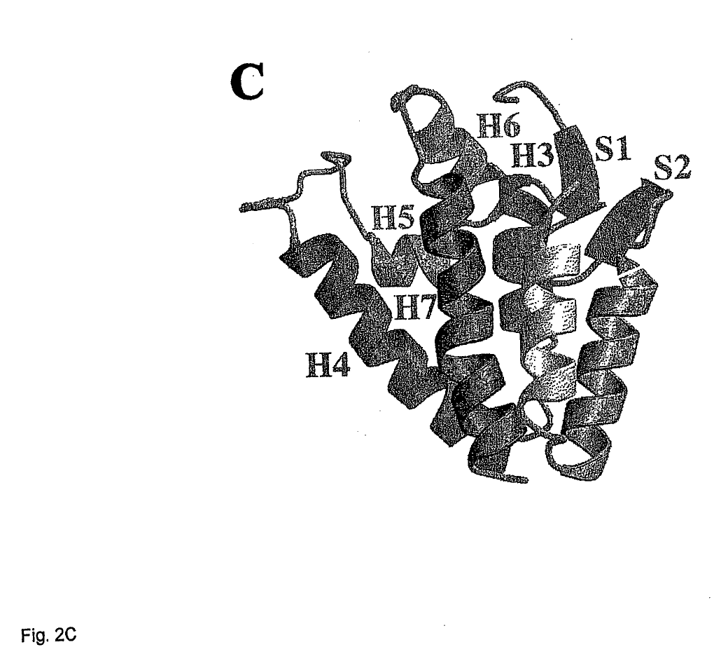

Mutational studies have shown that proteolytic processing at the N-terminus of CA is essential for viral replication. In addition, conformational changes at the N-terminus of CA upon proteolysis have been structurally characterized and are disclosed herein. Therefore, antiviral drugs can be based on the inhibition of CA conformational change. Disclosed is an assay that can be used to detect the CA conformational change and screen for small-molecule inhibitors by probing the accessibility of residues in different CA conformations (FIG. 2).

Also, a consequence of the Gag cleavage event is assembly of a central conical structure, termed the core, that is formed by the CA and NC proteins, as well as the viral RNA. This core structure is necessary for the assembly of infectious viral particles because mutations that block core formation inhibit infectious particle assembly. (e.g., see von Schwedler et al (1998) which is herein incorporated by reference for material related to assembly of the core and of infectious viral particle.) Another interaction that is necessary for infectious viral particle formation is dimerization between two CA proteins. If this dimerization is prevented the formation of infectious viral particle is inhibited. (Gamble et al. Science 278:849-853 (1997).

In vitro screening assays for the isolation of inhibitors of viral capsid maturation and in particular HIV viral capsid maturation, as well as for the isolation of inhibitors of core particle assembly and dimerization are needed so that these processes themselves can be studied and for the identification of additional HIV therapeutic agents. The disclosed compositions and methods disclosed herein address these needs.

Disclosed herein are the structures of proteins in which the final four MA residues were retained on the N-terminal domain of CA (₁₂₉MA-CA₂₇₈). The ₁₂₉MA-CA₂₇₈ structure differs significantly from that of the fully processed CA domain in that the N-terminal β-hairpin has rotated through ˜140° to pack against the protein's globular domain and the register between the first two helices has shifted by one helical repeat. In addition, the cationic half of a salt bridging interaction between CA Asp 183 and the N-terminus of the fully processed CA has been replaced by the protonated imidazole of His144. Overall, the structure of ₁₂₉A-CA₂₇₈ suggests how conformational flexibility at the CA N-terminus can result in the polymorphic CA assemblies observed in vitro and in vivo.

Also disclosed are structures of proteins that suppress the aggregation of the CA-NC protein so that lower concentrations of the protein can be used in in vitro viral assembly assays.

Disclosed are a variety of CA variants having various MA extensions. Disclosed herein it is shown that even short MA extensions cause significant rearrangement of the structural elements that surround the MA-CA junction and affect the structure of the N-terninal domain of CA. Furthermore, this rearrangement is similar to the rearrangement that takes place on maturation of the CA protein through proteolytic processing of the N-terminal end of CA.

Disclosed are methods and compositions which can be used in a CA maturation assay. For example, a high-throughput light scattering assay is disclosed which can be used to monitor CA maturation. In one embodiment of the method, a modified CA protein, as disclosed herein, and compounds from a chemical library can be added into a reaction mixture. The reaction mixture can be incubated for a period of time at a given temperature (for example overnight at 4° C.), and the amount of the modified CA protein which is reactive with a diagnostic reagent.is determined. The more reactive the CA protein is, the more likely molecules in the library are inhibitors of maturation. The initial library can be fractionated and re-tested in an iterative manner enriching for the molecules that inhibit assembly.

Screening for small molecule inhibitors of the CA conformational change using a high-throughput scintillation proximity assay (SPA) can be performed as follows. The following reagents will be added sequentially: 1) immature ₁₀₅MA-CA₂₇₈(His)₆ protein, 2) compounds from a chemical library, 3) HIV-1 protease, 4) [³] N-Ethylmaleimide (NEM), 5) Ni²⁺ SPA beads. Molecules that inhibit the CA conformational switch are expected to increase the light signal by enhancing the reactivity of CA₁₃₃₋₂₇₈(His)₆ protein with [³H]NEM.

The processing of the Gag molecule to form the infectious viral particle requires the assembly of distinct viral components including the CA and NC proteins as well as the viral RNA. This assembly forms a conical infectious core particle. Disclosed are methods and compositions which can be used in a CA-NC/DNA assembly assay. For example, a high-throughput light scattering assay is disclosed which can be used to monitor CA-NC/DNA assembly. In one embodiment of the method, CA-NC(G94D) protein, d(TG)₅₀ oligonucleotides, and compounds from a chemical library can be added into a reaction mixture. The reaction mixture can be incubated for period of time at a given temperature (for example overnight at 4° C.), and light scattering of the solution mixture will be performed and monitored for each reaction at for example, 312 nm. In this type of assay, inhibitors of CA-NC/DNA assembly which are present in the library will reduce the light scattering by reducing the cylinder formation of the CA-NC(G94D) protein. If there is a reduction in the light scattering, relative to controls, indicating that compounds in the library inhibit assembly, the initial library can be fractionated and re-tested in an iterative manner enriching for the molecules that inhibit assembly.

In addition, formation of the viral core structure requires that a CA dimer be formed. Inhibition of this dimerization leads to inhibition of viral infectivity. Also disclosed are compositions and methods for performing a CA dimerization assay. This assay allows for the screening and or testing of compounds for the inhibition of CA dimerization which then can be used as inhibitors for infectious viral particle formation. For example, a high-throughput scintillation proximity assay (SPA) can also be used in the CA dimerization assay. A typical reaction mixture can comprise: 1) anti-FLAG antibody-derivatized SPA beads, 2) (CA-CID)₂-FLAG protein, 3) ³H-(CA-CTD)₂, and 4) compounds from the chemical library. ³H-(CA-CTD)₂/(CA-CTD)₂-FLAG complex formation via dimerization will bring ³H into close proximity to the scintillant and give rise to a light signal. Inhibitors of CA dimerization will be detected via reduction of this light signal.

B. Compositions

Disclosed are compositions related to HIV-1 capsid protein and variants of the capsid protein. The disclosed variants of the capsid protein are characterized in that they can be assayed for whether, for example, amino acids in the approximately 600 cubic angstrom (˜600 Å³) cavity or whether amino acids associated with the unprocessed N-terminal tail of the capsid protein or whether amino acids in the alpha helix VI of the capsid protein are accessible to, for example, chemicals which can derivatize the accessible amino acids.

Also disclosed are compositions which interact with the ˜600 Å³ cavity of the capsid protein. These compositions can prevent the maturation of the capsid protein which can prevent infectious viral formation.

Disclosed are compositions comprising a modified CA protein, wherein the modified CA protein can be used to determine whether the ˜600 Å³ cavity of the modified CA protein is accessible.

Disclosed are compositions, wherein the modified CA protein comprises the amino acid sequence set forth in SEQ ID NO: 15 or a conserved variant or fragment thereof.

Disclosed are compositions, further comprising the amino acid sequence set forth in SEQ ID NO: 11.

1. CA Protein

The CA protein is typically a 230 residue polypeptide that is processed from the Gag polypeptide of HIV. The CA protein comprises two domains, an N-terminal domain and a C-terminal domain. The C-terminal domain is involved in correct viral packaging, Gag oligomerization, CA dimerization, and viral assembly. (Gamble et al., Science 1997) herein incorporated by reference for material related to the structure of the Gag polypeptide and the structure of the proteolytic products of the Gag polypeptide.).

Typically there can be two different numbering systems that reference CA. One is based on the Gag sequence and the position of CA in Gag. In Gag, CA typically contains residues from 133 to 363, the N-terminal domain of CA typically contains residues from 133 to 278, and the C-terminal domain typically contains residues from 278 to 363, for example. In the second numbering scheme, just the CA protein is referred to. In the CA numbering scheme, CA contains residues from 1 to 231, the N-terminal domain contains residues from 1 to 146, and the C-terminal domain contains residues from 146 to 231, for example.

The N-terminal and C-terminal domains of CA can be defined by the fimctions that each domain possesses which are discussed herein. For example, the C-terminal domain of CA could be considered the set of amino acids possessing the property of dimerization. It is understood that the precise point of where the C-terminal domain and the N-terminal domain intersect does not have to be a single amino acid. Rather the intersection can be considered a region. For example, the N-terminal domain can be considered to be defined by CA amino acids 1- 151 (corresponding to residues 133-283 in the unprocessed Gag polyprotein (Gamble, Cell 1996)), however the N-terminal domain can also be defined by amino acids 1-142, 1-143, 1-144, 1-145 or 1-146 or 1-147 or 1-148 or 1-149 or 1-150 or 1-151 or 1-152 or 1-153 or 1-154 or 1-155 or 1-156 ofthe CAprotein (133-363) SEQ ID NO: 1 for example. In other embodiments the N-terminal domain is defined by amino acids 1-144. (133-363) SEQ ID NO:1.

The N-terninal region also typically contains seven alpha helices. A CA protein that contains the seven alpha helices of the N-terminal domain, which are normally found in the first 145 N-terminal amino acids. However, as long as the seven alpha helices remain functional, N-terminal domains having less than 145 amino acids are contemplated. (Gamble et al., Cell 1996, herein incorporated by reference for material related at least to the structure of HIV proteins). The N-terminal domain could also be defined by the region containing the first seven alpha helices from the N-terminal end of the CA protein

The C-terminal domain is the region of the CA protein not defined as the N-terminal domain. Another way to define the C-terminal domain is by indicating that it can be amino acids 145-231 of the CA protein. In other embodiments the C-terminal domain can be defined as amino acids should it be 145-231 or 144-231 or 143-231 or 142-231 or 141-231 or 140-231 or 146-231 or 147-231 or 148-231 149-231 or 150-231 or 151-231 (133-363) SEQ ID NO:1 of the CA protein. The C-terminal domain of CA can also be defined as the amino acids residing on the C-terminal side of amino acid 140 or 141 or 142 or 143 or 144 or 145 or 146 or 147 or 148 or 14 9or 150or 151 or 152or 153or 154or 155(133-363)SEQIDNO:1 of the CA protein. The C-terminal domain can also be defined as the region of the CA protein that contains the 4 most C-terminal alpha helices of the CA protein.

Three-dimensional structures of the N-terminal domain of CA (CA₁₃₃₋₂₇₈) and the N-terminal domain of CA fused with the final four MA residues (₁₂₉MA-CA278) have been solved by NMR and X-ray crystallography (Gamble et al., 1996; Gitti et al., 1996; Stemmler et al., 2001) (incorporated by reference herein at least for material related to structure of HIV related proteins). Comparison of these structures reveals significant conformational changes at the N-terminal end of CA upon proteolysis, with the N-terminal β-hairpin and the surrounding helices 1, 3, and 6 oriented differently in CA₁₃₃₋₂₇₈ and ₁₂₉MA-CA₂₇₈. In ₁₂₉MA-CA₂₇₈, the N-terminal β-hairpin packs down against the globular domain of the protein. Upon removal of the MA residues, the hairpin projects away from the globular domain, allowing the new N-terminal Pro133 to form aburied salt bridge with side chain ofAsp183. The β-hairpin structure stabilized by salt bridge between Pro133 and Asp183 is important for mature particle formation, as mutation of Asp183 to Ala inhibits cylinder formation in vitro and blocks conical capsid assembly and viral replication in vivo (von Schwedler et al., 1998). Upon removal of the MA residues, the register between helices 1 and 2 also shifts by one helical repeat. cryoEM and image reconstruction of CA tubes reveals that CA hexamer, the building block of the mature viral capsid, is formed by six N-terminal domains of CA and stabilized by intermolecular packing of twelve helices 1 and 2 (Li et al., 2000). Therefore, the shift of register is thought to position the two helices correctly for CA hexamer formation and capsid maturation.

The CA protein is composed of two distinct domains. The elongated N-terminal domain (NTD) binds cyclophilin A (23-25) and plays an essential role in capsid formation, but is not absolutely required for immature particle formation (26). Nevertheless, point mutations within the domain can diminish particle formation, suggesting that the correct intermolecular packing interactions of the N-terminal domain of CA may contribute to Gag assembly (27). The globular C-terminal domain (CTD) of CA dimerizes in solution and in the crystal (28,29) and performs essential roles in both immature and mature particle assembly (30-32).

Studies of higher order structures formed by recombinant Gag and CA proteins have helped to define the structures and determinants of immature and mature HIV-1 particle assembly. In vitro, recombinant CA and CA-p2-NC can form long helical cylinders and cones that appear to be analogues of the mature viral capsid (33-40). Nucleic acid templates facilitate the assembly of constructs containing the NC domain, but are not absolutely required for either cylinder or cone formation (35, 38, 40). The CA and CA-NC tubes are composed of helices of CA hexamers, and image reconstructions and modeling analysis suggest that the CA NTD forms the hexameric rings and the CTD forms dimeric interactions that link the hexamers into a p6 surface lattice (40). These observations are consistent with the proposal that the conical viral capsid assembles following the principles of a “fullerene cone” (39) in which the body of the cone is composed of CA hexamers and the ends of the cone close via inclusion of a total of 12 pentameric defects. Thus, this model implies that the mature HIV-1 CA protein can form both hexameric and pentameric complexes that are analogous to their counterparts in complex icosahedral viruses⁴¹. The spherical immature HIV-1 particle is also an irregular object, although in this case the underlying lattice symmetry is not yet known (42-44).

One embodiment of the disclosed compositions involving the CA protein is shown in SEQ ID NO: 1 which discloses a particular variant of the CA protein. There are hundreds of variations of the CA protein. The Los Alamos National Laboratory, for example, keeps a comprehensive database of all of the known HIV variants, not only of CA protein, but of the entire HV genome. This database can be accessed by the public at Los Alamos Data base: http://hiv-web.lanl.gov/ and the material related to the HIV variant sequence, particularly variants related to CA protein are herein incorporated by reference. The regions of high homology, for example, “Major Homology Region” (MHR) can be readily identified in various sequences and strains of HIV. The MHR is the most conserved sequence in CA, and is a stretch of 20 amino acids, from residues 152 to 171 (or the corresponding residues in a variant or other HIv strain) in CA (SEQ ID NO: 11, IRQGPKEPFRDYVDRFYKTL). It has approximately 90% identity (without allowing for conservative mutations) amongst sequences of HIV-1 and HIV-2. Furthermore there are numerous other repositories of this type of information which are readily available to the skilled artisan. The disclosed compositions in certain embodiments include all known variants of the CA domain, in so far as each variant is capable of forming the approximately 600 cubic angstrom (˜600 Å³) cavity and can be used in or be the basis of a protein which can be used in the disclosed methods. Also, the disclosed compositions in certain embodiments include all known variants of the CA domain, in so far as each variant is capable of dimerizing or assembling in the disclosed assembly methods. Each of the specific known CA-domain variants is expressly described herein by reference to the Los Alamos database. It is understood that while the modified CA proteins disclosed herein include particular preferred embodiments, all functional CA proteins are disclosed herein.

a) CA-protein dimerization

Mutations that inhibit dimerization also inhibit viral replication. Mutations of amino acids trp184 or met 185 to ala resulted in a loss of dimerization with a reduction in viral replication. Ganser et al. Science, 1999, 283:80-83 which is herein incorporated by reference for material related to the structure of the CA C-terminal domain.). Further structural analysis of the CA C-terminal domain (CA-CID) has provided significant insight into particular amino acids involved in the dimerization of the CA-CTD. Worthylake et al. Acta Cryst. Biological Crystallograpy 1998 D55:85-92 which is herein incorporated by reference at least for material related to the structure of the capsid protein dimerization domain.

b) Protein Variants

As discussed herein there are numerous variants of the HIV-1 CA protein that are known and herein contemplated. In addition, to the known functional HIV-1 strain variants there are derivatives of the capsid and nucleocapsid proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well kiown, for example M13 primer mutagenesis. Amino acid substitutions are typically of single residues; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary MRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutainine | Gln | Q |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acid | pGlu | |
| serine | Ser | S |
| threonine | Thr | T |
| tyrosine | Tyr | Y |
| tryptophan | Trp | W |
| valine | Val | V |

TABLE 2
Amino Acid Substitutions

| | Exemplary Conservative Substitutions, |
|---|---|
| Original Residue | others are known in the art. |
| Ala | ser |
| Arg | lys, gln |
| Asn | gln; his |
| Asp | glu |
| Cys | ser |
| Gln | asn, lys |
| Glu | asp |
| Gly | ala |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed nucleic acids and proteins herein is through defining the variants and derivatives in terms of homology to specific known sequences. For example, SEQ ID NO:3 and 12 set forth a particular sequence of a modified CA protein and SEQ ID NO: 15 sets forth a particular sequence of a CA-CTD and SEQ ID NO: 18 sets forth a particular sequence of a capsid-nucleocapsid (CA-NC) protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids including, degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, a genus of sequences that can encode the protein sequence set forth in SEQ ID NO:3 is set forth in SEQ ID NO:4. SEQ ID NO:4 sets forth a population of sequences, all of which encode SEQ ID NO:3, that represents the degeneracy at the third position of each codon encoding each amino acid in SEQ ID NO:3. Each of these sequences is also individually disclosed and described. In addition, for example, a disclosed conservative derivative of SEQ ID NO:3 is shown in SEQ ID NO: 7, where the isoleucine (I) at position 6 is changed to a valine (V). It is understood that for this mutation all of the nucleic acid sequences that encode this particular derivative of the CA protein are also disclosed including for example, SEQ ID NO:8, which sets forth a population of sequences, all of which encode SEQ ID NO:7, that represents the degeneracy at the third position of each codon encoding each amino acid in SEQ ID NO:7.

As another example, one of the many nucleic acid sequences that can encode the protein sequence set forth in SEQ ID NO:15 is set forth in SEQ ID NO:23. Another nucleic acid sequence that encodes the same protein sequence set forth in SEQ ID NO:15 is set forth in SEQ ID NO:14 third position codons. SEQ ID NO:24 sets forth a population of sequences, all of which encode SEQ ID NO:15, that represents the degeneracy at the third position of each codon encoding each amino acid in SEQ ID NO:15. Each of these sequences is also individually disclosed and described. In addition, for example, a disclosed conservative derivative of SEQ ID NO:15 is shown in SEQ ID NO: 25, where the isoleucine (I) at position 6 is changed to a valine (V). It is understood that for this mutation all of the nucleic acid sequences that encode this particular derivative of the CA-CTD are also disclosed including for example SEQ ID NO:26 and SEQ ID NO:27, which sets forth a population of sequences, all of which encode SEQ ID NO:25, that represents the degeneracy at the third position of each codon encoding each amino acid in SEQ ID NO:25.

It is also understood that while no amino acid sequence indicates what particular DNA sequence encodes that protein within an organism, where particular variants of a disclosed protein are disclosed herein, the known nucleic acid sequence that encodes that protein in the particular strain of HIV from which that protein arises is also known and herein disclosed and described.

2. Compositions for Determining Structural State

Disclosed are compositions modified CA proteins which can be used to assess the conformation state of the CA protein.

NMR structures have revealed that the conformation of the N-terminal domain of CA changes dramatically when four MA residues are added to its N-terminus. These two CA conformations (CA₁₃₃₋₂₇₈ and ₁₂₉MA-CA₂₇₈) differ primarily in the orientations of the N-terminal β-hairpin and the surrounding helices 1, 3, and 6. In addition, a prominent cavity (˜600 Å³) in the structure of ₁₂₉A-CA₂₇₈ is filled in the structure of CA₁₃₃₋₂₇₈ by the new N-terminus formed upon removal of the MA residues. Disclosed are assays and compositions which determine whether small molecules bind in the cavity and block the conformational change. To screen for small-molecule inhibitors of the structural transition, a chemical probing assay is disclosed that can differentiate between CA in its two conformations.

The N-terminal P-hairpin packs down against the globular domain in the ₁₂₉MA-CA₂₇₈ structure, whereas it springs up and packs against helix 6 in the CA₁₃₃₋₂₇₈ structure. As a result, several residues in helix 6 are more exposed in the ₁₂₉MA-CA₂₇₈ structure.

Disclosed are compositions which take advantage of the exposure of helix 6 in the immature structure relative to the exposure of helix 6 in the mature structure.

Disclosed are compositions which comprise amino acids at the N-terminal end of a mature CA protein, such as CA₁₃₃₋₂₇₈ a version of which is set forth in amino acids 133-278 of SEQ ID NO:1. For example, disclosed are isolated molecules comprising a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid addition to the N terminus of CA₁₃₃₋₂₇₈, such as a four base extension in ₁₂₉MA-CA₂₇₈ or a 29 base extension in ₁₀₄MA-CA₂₇₈ Often these additional amino acids will be the amino acids or conserved variations which occur in the Gag sequence which is N-terminal to the CA₁₃₃₋₂₇₈. It is also understood that N-terminal extensions, including extensions up to the beginning of MA can also be used.

These compositions, for example, can be residues in or near helix 6 which are reactive to various reagents. For example, cysteines are reactive with many reagents which react with a free thiol. The CA protein in SEQ ID NO:1, for example, does not have a cysteine residue in the helix 6 region. Thus, compositions, comprising the CA₁₃₃₋₂₇₈ structure with one of the amino acids within or near the helix 6 substituted with a cysteine residue are disclosed (for example, see the protein set forth in SEQ ID NO:12 and SEQ ID NO:14). Helix 6 residues are typically amino acid residues from about 244 to about 252 of SEQ ID NO:9. Thus disclosed are CA compositions that comprise a cysteine substitution at one of amino acid residues about 242 to about 255 of SEQ ID NO:9 or the analogous position in another CA variant. Those of skill in the art can readily determine which residues are related to the helix 6 residues.

Also disclosed is the substitution of any residue that is more exposed in the immature conformation than in the mature conformation.

It is also recognized that as significant structural changes take place between the immature and mature conformations, residues that are disclosed here which are more exposed in the mature conformation than in the immature conformation can also be substituted with cysteines.

It is understood that there are other chemically reactive amino acids, for example methioneine, which can also be used as a substitute. It is also understood that more than one reactive substitution can be made in a given composition.

For example, disclosed are compositions that have had the Ile247 substituted to a Cys in CA helix 6. For example, disclosed are ₁₀₅MA-CA₂₇₈ compositions and CA332-278 compositions that have had a cysteine substitution within the helix 6, for example, at position 247 of SEQ ID NO:9.

If a cysteine is the substituted amino acid (or happens to be within a naturally occurring variant) a variety of reagents can be reacted with the cysteine. For example, ³H-N-ethyl maleimide can be used and the amount of ³H incorporated into the CA molecule is analyzed. Any reagent that can react with a cysteine can be used by those of skill in the art.

Disclosed is a composition comprising a modified form of the HIV-1 CA protein, wherein the modified form allows for detection of conformational changes that take place in the modified form of the protein. These conformational changes are related to the conformational changes that take place during maturation of the CA protein.

In some embodiments the composition comprises the HIV-1 modified CA protein which comprises the amino acid sequence set forth in SEQ ID NO: 3 or SEQ ID NO:12 or a conserved variant thereof or fragments thereof.

The modified CA protein can be formed in a number of ways. What is required, typically, is that the modified form facilitate the determination of whether the ˜600 Å³ cavity in the structure of ₁₂₉MA-CA₂₇₈ is occupied by a molecule, such as a small molecule. This determination can be made by, for example, observing the differential accessibility of amino acids making up the N-terminal domain of the modified CA protein or making up the ˜600 Å³ cavity of the modified CA protein. For example, by mutating Ile247 to Cys, which is in the CA protein and which is understood to be within alpha helix VI of the CA protein, an amino acid which can be reacted with reagents sensitive to sulfur can be used. As amino acid 247 of the CA protein can either be accessible to reagent or not accessible to reagent correlated to the occupation of the ˜600 Å³ cavity the level of chemical modification that occurs at the Cys247 of the CA protein correlates with the extent to which the ˜600 Å³ cavity is occupied or not occupied. A greater chemical modification of amino acid 247 indicates less occupation of the ˜600 521 ³ cavity and a lesser chemical modification indicates a greater occupation of the ˜600 Å³ cavity.

Typically the finctional requirement of the modified CA proteins is that they have properties which allow for the determination of whether the ˜600 Å³ cavity is occupied by the N-terminal amino acids of the CA protein. It is understood that occupied does not mean static or constant, but rather indicates that the ˜600 Å³ cavity over time is filled. This is understood to be a continuum from over time it is never filled to over time it is always filled. Typically, the extent the ˜600 Å³ cavity is occupied is a relative average over time of how much the ˜600 Å³ cavity was occupied.

The CA protein can be any CA protein or protein fragment or conserved variant of the CA protein which is capable of determining whether the ˜600 Å³ cavity is occupied. It is understood that the variants of the CA protein refer to the known HIV alleles within the CA protein and non-natural variants of the CA protein that retain the ability to form the ˜600 Å³ cavity. The ˜600 Å³ cavity comprises a number of amino acids. Pro 133 is part of the ˜600 Å³ cavity as well as Val-135, His-144, Gln-145, Ile-147, Ser-148, and Thr-151, Trp-155, Phe-172, Leu-175, Ser-176, and Ala-179. These residues are generally well ordered in the structure and conserved in other HIV-1 isolates (Los Alamos database).

It is understood that the CA protein can be modified by for example having one or more molecules attached to it, for example, other protein molecules that can be useful in detection of the occupation of the ˜600 Å³ cavity. These detection molecules may be, for example, finction in a pair, such as a ligand or hapten, that binds to or interacts with another compound, such as a ligand-binding molecule or an antibody. Preferred indirect linker pairs include for example biotin and streptavidin or avidin which can be incorporated into proteins. A preferred hapten for use as one part of an indirect linker is digoxygenin (Kerkhof, Anal. Biochem. 205:359-364 (1992)).

Methods for attaching molecules, particularly protein based molecules, to other proteins, such as the CA protein are well established. Attachment can be accomplished by attachment, for example, to aminated groups, carboxylated groups or hydroxylated groups using standard attachment chemistries. Examples of attachment agents are cyanogen bromide, succinimide, aldehydes, tosyl chloride, avidin-biotin, photocrosslinkable agents, epoxides and maleimides. A preferred attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992) both of which are herein incorporated by reference for at least material related to protein derivatization.

One way of attaching proteins is through free amino groups present on the proteins. Proteins can be coupled by chemically cross-linking a free amino group on the protein to reactive side groups present within the linker. For example, proteins may be chemically cross-linked to linkers that contain free amino or carboxyl groups using glutaraldehyde or carbodiimides as cross-linker agents. In this method, aqueous solutions containing free proteins, such as CA units, are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

For example, disclosed are CA compositions that comprise a histidine tag, comprising 6 histidine residues. For example, disclosed are compositions ₁₀₅MA-CA₂₇₈(His)₆ and CA₁₃₃₋₂₇₈(His)₆.

3. Compositions that Inhibit Maturation

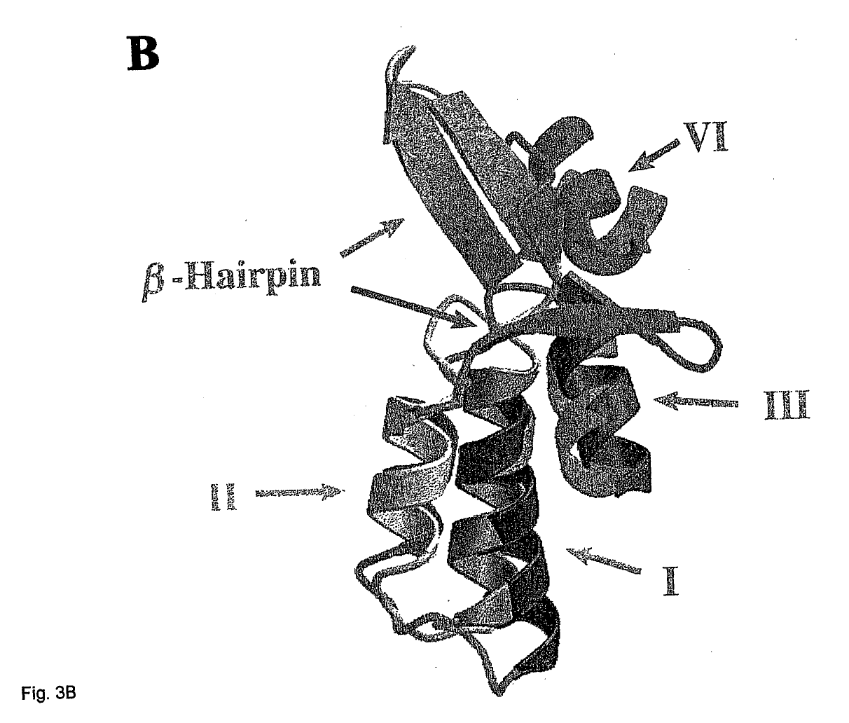

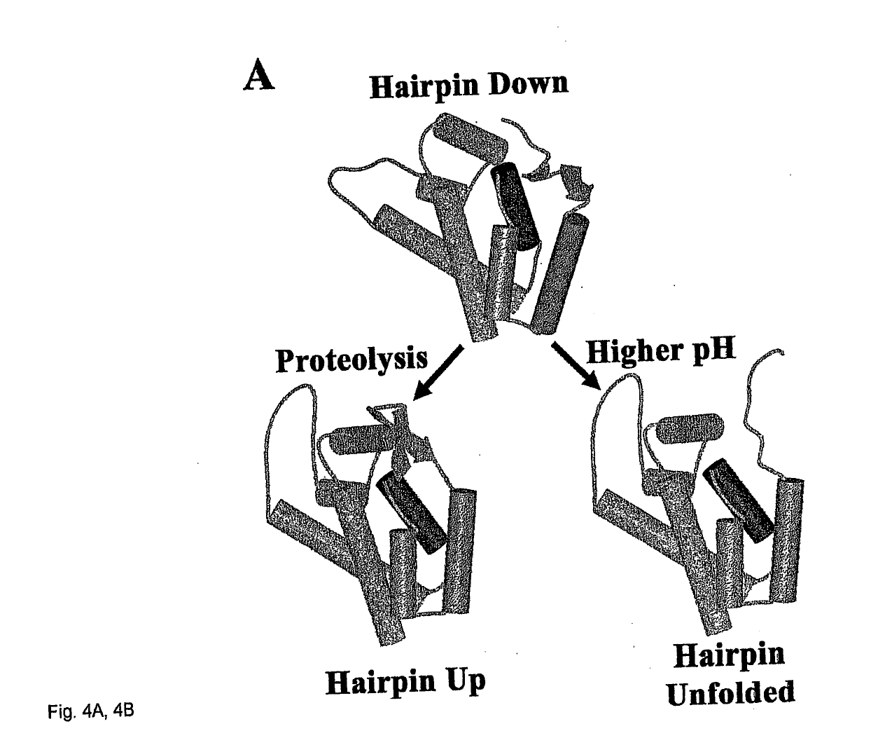

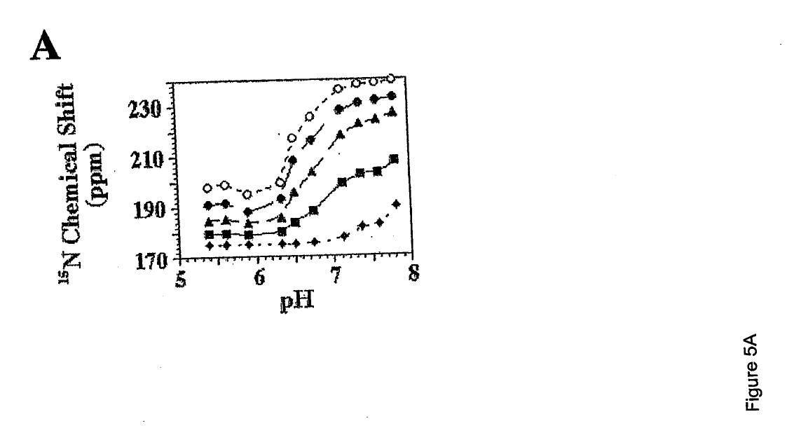

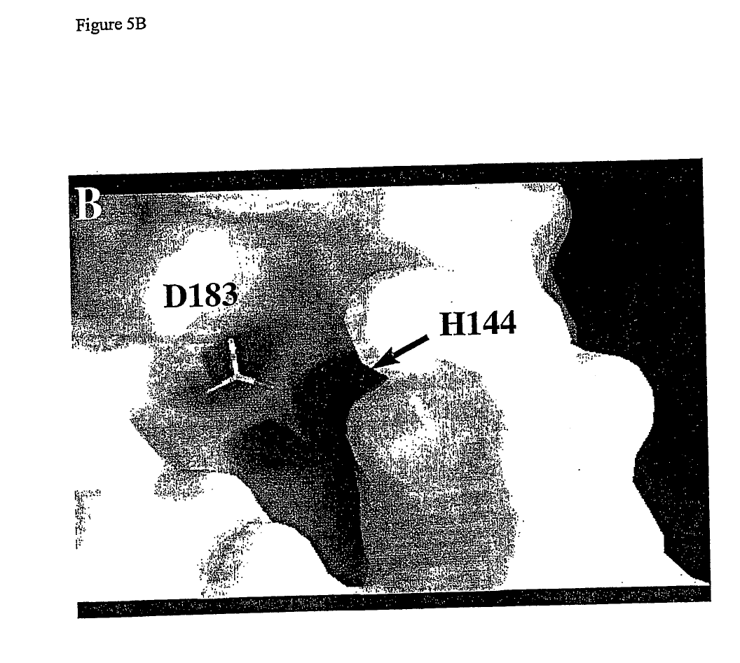

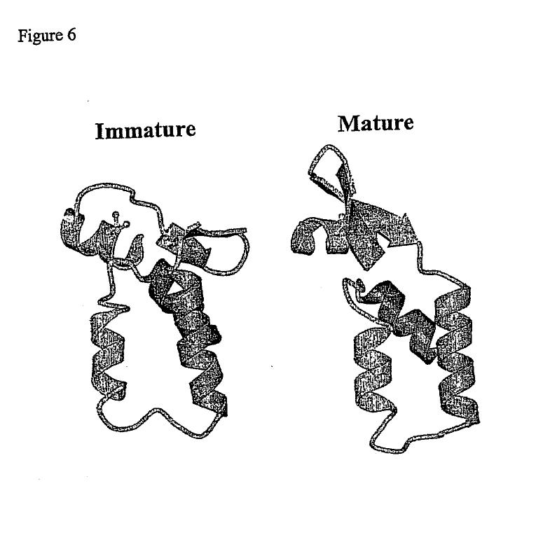

Capsid assembly can be altered by small molecules that bind specifically and stabilized the hairpin down conformation. The surface topology of the protein exhibits at least two unique cavities that are possible binding sites for such small molecule inhibitors. The larger (˜600 Å³), corresponds to the approximate binding site for Pro 133 in the hairpin up conformation (FIG. 5B). The His144 Asp183 salt bridge forms the base of this cavity, and the other residues that define the walls are generally well conserved in different HIV isolates. Thus the size, conservation, and apparent functional importance of the cavity make it a target for inhibitor design.

The disclosed compositions which can be used in the disclosed methods are based on the CA protein. The CA protein undergoes maturation and during this process there is a stage where the N-terminal amino acids of the CA protein interact with the ˜600 Å³ cavity of the CA protein. When the N-terminal amino acids interact with the ˜600 Å³ cavity of the CA protein amino acids that make up the N-terminal sequence are protected from either chemical or enzymatic manipulation. Therefore, this chemical probe assay can be used to detect the CA conformational change and can be used to isolate molecules that interact with the ˜600 Å³. Furthermore, the assay can be adapted for high-throughput screening of small molecules that inhibit the structural transition.

Disclosed are compositions that inhibit the maturation of the CA protein produced by the process of screening for interaction with the ˜600 Å³ cavity of the CA protein. Disclosed are products produced using the disclosed methods that use any of the disclosed compositions herein. For example, disclosed are compositions that inhibit the maturation of the CA protein produced by the process of screening for interaction with the ˜600 Å³ cavity of the CA protein wherein the screening is performed with a CA protein reactive in helix 6 with chemical reagent. For example, disclosed are products produced by the disclosed screening methods, wherein the helix 6 of the CA protein used in the screening method comprises a cysteine residue.

Disclosed are compositions which interact with the the ˜600 Å³ cavity of the CA, such as the amino acids of the cavity. Also disclosed are compositions that interact with Pro 133 or Asp 183. Disclosed are compositions that interact with Pro 133 or Asp 183 and prevent the formation of a salt bridge between Pro 133 and Asp 183. Disclosed are compositions that prevent the formation of a salt bridge between Pro 133 and Asp 183. Also disclosed are compositions that interact with Pro 133 or Asp 183 and/or prevent the formation of a salt bridge between Pro 133 and Asp 183 which are isolated using the disclosed modified CA proteins disclosed herein and the screening methods based on the modified CA proteins disclosed herein.

4. Modified CA Protein Dimers

The CA protein as discussed herein is capable of dimerizing. A dimer of a native CA protein thus comprises two CA molecules that are interacting with each other. The disclosed compositions are based on taking at least two CA units and stabilizing an association between the CA units such that a modified CA dimer is formed. These stabilized CA dimers are capable of themselves interacting with at least one more CA molecule, particularly if the modified CA dimer is formed at least in part through interactions not based on the dimerization domain of the CA protein. Thus, the disclosed compositions are “CA dimers” in so far as they comprise at least two CA molecules, but the formation of the modified CA dimers occurs such that the stability of the modified CA dimer is greater than the stability of the natural dimer of CA proteins formed through the dimerization domains of the CA proteins.

Disclosed is a composition comprising a modified dimer of the HIV-1 CA carboxy terminal domain (CA-CTD), wherein the dimer is more stable than the dimer naturally.

In certain embodiments the modified dimer has a Kd for dimer formation of less than 20 μM or 10 μM or 5 μM or 2.5 μM or 1 μM or 500 nM or 250 nM or 100 nM or 10 nM or 1 nM.

In other embodiments the modified dimer further comprises two HIV-1 CA carboxy terminal domains in tandem.

In some embodiments the dimer comprises the HIV-1 CA carboxy terminal domain which comprises the amino acid sequence set forth in SEQ ID NO: 15 or a conserved variant thereof.

In certain embodiments the dimer further comprises the amino acid sequence set forth in SEQ ID NO: 16 which adds the flag sequence, DYKDDDDK.

The modified CA dimers can be formed in a number of ways. What is required is that at least two CA monomers or CA-CTD monomers that form the modified CA dimer or modified CA-CTD dimer be connected in such a way that the connection between the monomers forming the modified dimer is stronger than the connection that would be formed between the monomers through the natural dimerization domain of the CA or CA-CTD monomers. For example, the CA or CA-CTD monomers can be covalently linked together by for example a small stretch of amino acids or a chemical linkage such as a disulfide linkage. The monomers can also be linked together via non-covalent interactions between for example a biotin that is attached to one monomer and a streptavidin protein that is conjugated to another monomer. Also disclosed herein are heterogenous combinations of these disclosed compounds, such as a linker composed of amino acids and a disulfide linkage.

The functional requirement of the modified dimers is that they are formed with a K_{d} that is smaller than the K_{d} of natural dimerization. In certain embodiments the modified dimers must be formed with a K_{d} less than 40 μM or 20 μM or 10 μM or 5 μM or 2.5 μM or 1 μM or 500 nM or 250 nM or 100 nM or 10 nM or 1 nM or 0.1 nM or 0.01 nM.

In preferred embodiments the monomers are covalently linked together forming the modified dimer. In certain embodiments the modified dimer is formed by covalently attaching two CA protein monomers or CA-CTD monomers together in tandem through an amino acid linker. It is understood that this linker can be any amino acid sequence and in fact can be any amino acid sequence lining, for example to CA residue Ala217, (or beyond) and ranging in length from 0 to 50 amino acids long, as long as the requirements set forth herein are maintained. In more preferred embodiments the dimer is defined by the sequence set forth in SEQ ID NO:16 or a conserved variant thereof. The CA-CTD monomers of this composition have the sequence set for in SEQ ID NO:15 or a conserved variant thereof and are connected together via a two amino acid connector having the sequence, PW.

Another preferred embodiment is the modified dimer set forth in SEQ ID NO:17 or a conserved variant thereof This dimer construct contains the same dimer construct set forth in SEQ ID NO:16, however, a Flag sequence (SEQ ID NO:22) has been added. The Flag sequence allows a scintillation proximity assay to be performed during a method of screening for inhibitors of dimerization as a detection step.

a) CA-L-CA

A general way of describing the modified dimers is that the modified dimers have the structure CA-L-CA. Each of these parts is discussed in detail herein.

(1) CA

The CA portion of the structure can be any CA protein, CA protein variant, CA protein derivative, CA-CTD, CA-CTD variant, or CA-CTD derivative capable of forming a CA dimer. It is understood that the variants of the CA protein and CA-CTD protein refer to the known HIV alleles within the CA CTD domain. A derivative of the CA protein or CA-CTD includes non-natural derivatives of the CA protein and CA-CTD that retain the ability to dirnerize.

In some embodiments the CA portion of the structure can be any CA protein, CA protein variant, CA-CTD, or CA-CTD variant capable of forming a CA dimer.

(2) Linker

The part of the structure designated by L can be any molecule or combination of molecules that cause any two CA units to interact with a stability greater than the stability of natural CA protein dimerization. L can be a variety of molecules including macromolecule(s) such as amino acid(s), chemical linkers such as polyethyleneglycol (PEG) and indirect linkers such as a biotin-streptavidin pair. Also disclosed herein are heterogenous combinations of these disclosed compounds, such as a linker composed of amino acids and PEG.

One preferred way of defining the linker is by defining the length of the linker. While the linker can be any length that allows the CA units and the modified CA or CA-CTD dimer to function as described in some embodiments the linker is less than 360 Å or 300 Å or 250 Å or 200 Å or 150 Å or 100 Å or 75 Å or 50 Å or 36 Å or 30 Å or 25 Å or 20 Å or 15 Å or 10 Å or 9 Å or 8 Å or 7 Å or 6 Å or 5 Å or 4 Å or 3 Å or 2 Å or 1 Å or 0 Å. Those of skill in the art can easily determine the length of any linker that is used in the disclosed compositions or methods.

(a) Amino Acids

If L is an amino acid or amino acids it preferably will be less than 50 or 25, 24, 23, 22, 21, 20, 19, 18, 17, 16, 15, 14, 13, 12, 11, 10, 9, 8, 7, 6, 5, 4, 3, or 2 amino acids long. The sequence of the linker can be any sequence that does not prohibit the dimerization domains of the CA units from dimerizing with a CA protein or CA-CTD. Preferred linker sequences are PW, or sequences that are rich in glycine. Glycine becomes more preferred as the linker increases in length. Sequences that are rich in glycine, proline, and serine are preferred to minimize unwanted secondary structure. Thus, when amino acids are used as the linker, the modified CA dimers in essence can function as a fusion protein and can be made through standard recombinant biotechnology techniques.

(b) Chemical Linkers

As used herein, “chemical linker” or “linker” means a flexible, essentially linear molecular strand. Preferably, the molecular strand comprises a polymer. Most preferably, the polymer strand has at least two finctionalized ends. After undergoing chemical bonding with the compounds to be linked, the linker residue may be referred to as a “chemical tether,” “molecular tether,” or “tether.”

As used herein, the term “soluble” refers to an article which, upon contacting with an appropriate liquid at an appropriate temperature, dissolves partially or completely into the liquid to form a solution. As used herein and in the claims, the term “dispersable” refers to an article which, while not necessarily “soluble,” is subject to structural weakening and breakup when subjected to a suitable liquid at a suitable temperature.

As used herein, the term “alkyl” is used to designate a straight or branched chain substituted or unsubstituted aliphatic hydrocarbon radical containing from 1 to 12 carbon atoms. As used herein and in the claims, the term “aryl” is used to designate a substituted or unsubstituted aromatic hydrocarbon radical. Aliphatic and aromatic hydrocarbons include both substituted and unsubstituted compounds, wherein the substitution can occur in the backbone or pendent groupings of the hydrocarbon.

As used herein, the term “functionalized” means having a chemically reactive moiety capable of undergoing a chemical reaction.

As used herein, the term “hydrophilic” means having an affinity for water; that is, hydrophilic compounds or functionalities are soluble, or at least dispersable, in water.

The L can also be any standard chemical linker, such as PEG or molecules similar to PEG. Typically the chemical linkers will be water soluble carbon based linkers.

One type of chemical linker is a sulfur based linker which can form disulfide bonds with Cys contained in the CA units. The Cys contained within the CA units can either be native or can be engineered onto for example, the carboxy terminal portion of the CA unit.

While any polymer strand may be used as a chemical linker, hydrophilic polymers are preferred linkers. Also, it is preferred that the strand, after linking, is inert toward the compounds that are linked thereby.

Examples of hydrophilic polymers suitable as linkers include polyethylene glycol (PEG), polypropylene glycol (PPG), polysaccharides, polyamides (nylon), polyesters, polycarbonates, polyphosphates, and polyvinyl alcohol. Most preferred is polyethylene glycol.

Examples of other polymers that can be used as linkers include hydrocarbons such as polyethylene and polypropylene, polymethacrylic acids, and polysiloxanes.

Copolymers containing moieties found in the above polymers are also suitable as linkers; examples include poly(ethylene-co-vinyl alcohol) and poly(propylene-co-vinyl alcohol).

Various substituents can also be incorporated into the polymer (within the backbone or on pendant groups) or complexed with the polymer to affect the properties of the polymer (e.g. solubility).

The length of the chemical linker typically would be less than 200 or 150 or 100 or 90 or 80 or 70 or 60 or 40 or 30 or 20 or 10 or 5 units in length.

(c) Indirect Linkers

When the L is formed by an indirect linker, it is typically composed of two molecules, which interact with each other in a specific way. Typically one of these molecules would be attached to one CA unit and the cognate molecule would be attached to another CA unit and the interaction of the two molecules forming the indirect linker would bring the two CA units in close proximity.

Typically, it is preferred that the indirect linkers form strong linkages. In certain embodiments the linkage has a K_{d} more than 10 fold, 100 fold, 1,000 fold, 10,000 fold, 100,000 fold, or 1,000,000 fold lower than the K_{d} of natural dimerization of a CA unit. In certain embodiments the linkage itself has a K_{d} of less than 100 nM, 10 nM, 1 nM, 100 pM, 10 pM, 1 pM, 100 fM, 10 fM, or 1 fM.

One example of an indirect linker pair is a compound, such as a ligand or hapten, that binds to or interacts with another compound, such as a ligand-binding molecule or an antibody. Preferred indirect linker pairs include for example biotin and streptavidin or avidin which can be incorporated into proteins. A preferred hapten for use as one part of an indirect linker is digoxygenin (Kerkhof, Anal. Biochem. 205:359-364 (1992)).

Methods for attaching molecules, particularly protein based molecules, to other proteins, such as the CA units are well established. Attachment can be accomplished by attachment, for example, to aminated groups, carboxylated groups or hydroxylated groups using standard attachment chemistries. Examples of attachment agents are cyanogen bromide, succinimide, aldehydes, tosyl chloride, avidin-biotin, photocrosslinkable agents, epoxides and maleimides. A preferred attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992) both of which are herein incorporated by reference for at least material related to protein derivatization.

One way of attaching proteins is through free amino groups present on the proteins. Proteins can be coupled by chemically cross-lining a free amino group on the protein to reactive side groups present within the linker. For example, proteins may be chemically cross-linked to linkers that contains free amino or carboxyl groups using glutaraldehyde or carbodiinides as cross-linker agents. In this method, aqueous solutions containing free proteins, such as CA units, are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

(d) Number

The disclosed compositions and methods in certain embodiments can have more than two CA units. Typically when there are more than two CA units there will always be one less linker than the total number of CA units. For example, if there were 5 CA units there would typically be 4 linker units. The combinations of CA units can be any combination of the different types of CA units. For example one could have a modified CA dimer with four CA units where two CA units were native CA proteins and two CA units were CA-CTD units. Furthermore, there can be any combination of linkers used. For example, if there were two linkers in a particular embodiment, one linker could be an amino acid linker and the other linker could be a PEG.

5. Inhibitor Library

The disclosed methods in some embodiments involve the use of chemical or combinatorial libraries to search for inhibitors of the occupation of 600 Å³ cavity, for example, inhibitors of the of the occupation of ˜600 Å³ cavity in the construct set forth in, for example, SEQ ID NO:3 or 5 or 12. The disclosed methods in some embodiments involve the use of chemical or combinatorial libraries to search for inhibitors of dimerization of CA units or inhibitors of assembly of the cone or conical assembly formation. Any type of chemical or combinatorial library which contains molecules which may inhibit the occupation of ˜600 Å³ cavity, for example, inhibitors of the of the occupation of 600 Å³cavity in the construct set forth in SEQ ID NO:3 or 5 or 12 or which inhibit CA dimerization or cone or conical formation, can be used in the present methods.

Typically libraries contain macromolecules, such as proteins, nucleic acids, or various sugar based macromolecules, or the libraries contain small molecules that are based on any workable functionality, such as carboxcylic acids, esters, amides, pyrimidinediones; benzodiazepindiones, benzofurans, indoles, or morpholinos, dihydrobenzopyrans, sulfonamides, substituted and unsubstituted heterocyclics, pyrimidines, purines, carbohydrates, conjugated systems and conjugated ring systems, and other moieties capable of directed synthesis leading to complex mixtures of compounds.

Libraries which contain molecules that can be used in the disclosed methods are well know in the art. For example, libraries and methods are disclosed in, for example, de Julian-Ortiz JV, “Virtual Darwinian drug design: QSAR inverse problem, virtual combinatorial chemistry, and computational screening,” Comb Chem High Throughput Screen. 2001 May;4(3):295-310; Chauhan PM, Srivastava SK, “Recent developments in the combinatorial synthesis of nitrogen heterocycles using solid technology,” Comb Chem High Throughput Screen. February 2001;4(1):35-51; Huc I, Nguyen R, “Dynamic combinatorial chemistry, Comb Chem High Throughput Screen. February 2001;4(1):53-74; Barkley A, Arya P, “Combinatorial chemistry toward understanding the function(s) of carbohydrates and carbohydrate conjugates, Chemistry. 2000 Feb. 2;7(3):555-63; Curran D P, Josien H, Bom D, Gabarda A E, Du W, “The cascade radical annulation approach to new analogues of camptothecins,” Combinatorial synthesis of silatecans and homosilatecans, Ann N Y cad Sci. 2000;922:112-21. 21; Houghten R A., “Parallel array andmixture-based synthetic combinatorial chemistry: tools for the next millennium, “Annu Rev Pharmacol Toxicol. 2000;40:273-82; Weber L., “High-diversity combinatorial libraries, Curr Opin Chem Biol.2000 June;4(3):295-302; Bohm H J, Stahl M., “Structure-based library design: molecular modeling merges with combinatorial chemistry,” Curr Opin Chem Biol. 2000 June;4(3):283-6; Floyd C D, Leblanc C, Whittaker M., “Combinatorial chemistry as a tool for drug discovery,” Prog Med Chem. 1999;36:91-168; 45: Nestler H P, Liu R., “Combinatorial libraries: studies in molecular recognition,” Comb Chem High Throughput Screen. 1998 October;1(3):113-26; 48: Kirkpatrick D L, Watson S, Ulhaq S., “Structure-based drug design: combinatorial chemistry and molecular modeling,” Comb Chem High Throughput Screen. 1999 August;2(4):211-21; Furka A, Bennett W D, “Combinatorial libraries by portioning and mixing,” Comb Chem High Throughput Screen. 1999 April;2(2):105-22; Schweizer F, Hindsgaul O., “Combinatorial synthesis of carbohydrates,” Curr Opin Chem Biol. 1999 June;3(3):291-8; and Oliver S F, Abell C., “Combinatorial synthetic design,” Curr Opin Chem Biol. 1999 June;3(3):299-306 all of which are herein incorporated by reference for at least material related to combinatorial libraries and methods and synthesis and use of the same.

Chemical libraries and methods of using the same are also disclosed in, for example, U.S. Pat. No. 6,255,120 for “Combinatorial library of substituted statine esters and amides via a novel acid-catalyzed Rearrangement;” U.S. Pat. No. 6,207,820 for “Combinatorial library of moenomycin analogs and methods of producing same;” U.S. Pat. No. 6,168,912 for “Method and kit for making a multidimensional combinatorial chemical library;” U.S. Pat. No. 6,114,309 for “Combinatorial library of moenomycin analogs and methods of producing same;” U.S. Pat. No. 6,025,371 for “Solid phase and combinatorial library syntheses of fused 2,4-pyrimidinediones;” U.S. Pat. No. 6,017,768 for Combinatorial dihydrobenzopyran library;” U.S. Pat. No. 5,962,337 for Combinatorial 1,4-benzodiazepin-2,5-dione library;” U.S. Pat. No. 5,919,955 for “Combinatorial solid phase synthesis of a library of benzofuran derivatives;” U.S. Pat. No. 5,856,496 for “Combinatorial solid phase synthesis of a library of indole derivatives;” for U.S. Pat. No. 5,821,130 for “Combinatorial dihydrobenzopyran library;” U.S. Pat. No. 5,712,146 for “recombinant combinatorial genetic library for the production of novel polyketides;” for U.S. Pat. No. 5,698,685 for “Morpholino-subunit combinatorial library and method;” U.S. Pat. No. 5,688,997 for “Process for preparing intermediates for a combinatorial dihydrobenzopyran library;” and U.S. Pat. No. 5,618,825 for Combinatorial sulfonamide library” all of which are herein incorporated by reference for at least material related to combinatorial libraries and methods and synthesis and use of the same.

The disclosed methods can be used to test any number of compounds contained within a given combinatorial library.

C. Methods

Disclosed are methods of using compositions comprising a modified CA protein, wherein the modified CA protein can be used to determine whether the ˜600 Å³ cavity of the modified CA protein is accessible.

Disclosed are methods, wherein the modified CA protein comprises the amino acid sequence set forth in SEQ ID NO: 15 or a conserved variant or fragment thereof.

Disclosed are methods wherein the composition, further comprises the amino acid sequence set forth in SEQ ID NO: 11.

Disclosed are methods of screening for molecules that inhibit maturation of HIV-1 CA protein comprising interacting a target molecule with the modified HIV-1 CA protein, forming a molecule-HIV-1 CA mixture and collecting the molecules that reduce the occupation of the ˜600 Å³ cavity of the modified CA protein.

Disclosed are methods of screening for molecules that inhibit maturation of HIV-1 CA protein comprising (a) interacting a target molecule with a modified HIV-1 CA protein as disclosed herein, forming a molecule-HIV-1 CA mixture, (b) removing unbound molecules, (c) determining whether the cysteine at position 247 of SEQ ID NO:12 or SEQ ID NO:14 is reactive and (d) collecting the molecules that make the cysteine at position 247 reactive.

Disclosed are methods, further comprising the step of repeating steps a-d with the collection of carboxy terminal domain molecules.

Disclosed are methods of screening for molecules that inhibit the N-terminal domain of a CA protein comprising forming a mixture of the CA protein and a target molecule making a modified CA protein-target molecule solution, and determining the reactivity of an amino acid in helix VI of the modified CA protein.

Disclosed are methods of testing a molecule for the potential to inhibit HIV-1 capsid maturation comprising incubating the molecule with a modified HIV-1 CA protein comprising a ˜600 Å³ cavity of the modified CA protein, and determining whether the molecule binds the ˜600 Å³ cavity of the modified CA protein.

Disclosed are methods, wherein the CA protein comprises SEQ ID NO:1 or a conserved variant or fragment thereof Disclosed methods, wherein the modified CA protein comprises substitution of amino acid of the I at position 115 of SEQ ID NO:1 or a conserved variant or fragment thereof.

Disclosed are methods, wherein the mutation produces a cysteine at position 115 of SEQ ID NO:1 or a conserved variant or fragment thereof.

It is understood that the disclosed methods can be performed by, for example, incubating the disclosed compositions with a possible inhibitor or a library of molecules, and then addition of the HIV protease can cause cleavage of the remaining CA-MA N-terminal amino acids, which would typically allow the CA protein to undergo a conformational change. If however, an inhibitor prevents this, the disclosed compositions can indicate the lack of a conformational change and this would indicate that a conformational change inhibitor was present in the assay. Those of skill in the art would understand how to perform appropriate controls, for example, showing that the inhibitor does not inhibit protease activity.

1. CA Capsid Maturation Assay

Disclosed are methods for testing a molecule for the potential to inhibit HIV-1 capsid maturation comprising incubating the molecule with a modified HIV-1 CA protein, and determining whether the molecule inhibits ˜600 Å³ cavity occupation in vitro. The HIV-1 CA protein can be produced using any recombinant biotechnology or synthetic method.

Disclosed are methods that utilize modified CA proteins that have amino acids in the alpha VI helix which are reactive and their reactivity can be assayed. Any naturally occurring variations of the CA protein which possess such amino acids can also be used in the disclosed methods.

Disclosed are screening assays for isolating inhibitors of the occupation of the ˜600 Å³ cavity. Such inhibitors can inhibit maturation of the CA protein. Typically the screening assay would be performed as a high-throughput, batch assay in which a chemical library would be screened (e.g., in a 384 well plate format) and light scattering can be used to monitor the occupation of the ˜600 Å³ cavity. For example, the modified CA protein can be used for determination of the occupation of ˜600 Å³ cavity, and compounds from the chemical library can be incubated together. The reaction mixture can be incubated overnight at 4° C., and light scattering at 312 nm measured for each reaction or incorporation of label, such as flourophore or radiolabel attached to a reactant with the modified amino acid or acids can be measured. Inhibitors of the occupation of ˜600 Å³ cavity will reduce the light scattering by reducing the cylinder formation or they will decrease the amount of incorporation of the label and thereby score as “positives” in the assay.

a) CA Protein

The disclosed methods can use the CA proteins disclosed herein. In some embodiments the modified CA protein comprises amino acids 133-433 of the HIV gag protein (denoted CA-NC). Preferred formns of the protein are those set forth in SEQ ID NOS: 1, 3, 5, and 12, particularly when these sequences contain a reactive amino acid in the helix VI region of the CA protein. A preferred form of the modified CA protein are forms derived from the HIV-1 strain NL4-3 . However, there are a very large number of HIV-1 strain variants, as discussed above, which can be found at for example the Los Alamos database which also produce CA-NC proteins that function in the disclosed methods.

As with the CA protein set forth in SEQ ID NO:1, and the representative nucleic acids that encode them set forth in SEQ ID NO:2, the nucleic acid sequences encoding the modified CA proteins disclosed herein are also described and disclosed, including all degenerate sequences. The nucleic acids encoding the disclosed and described variants of the modified CA protein including SEQ ID NOS: 3 and 5 are also disclosed including all degenerate sequences.

b) Reaction Conditions

Any reaction conditions that allow of the occupation of ˜600 Å³ cavity in constructs capable of doing this in the absence of an inhibitor or test molecule can be used for the disclosed assay. For example, the reaction conditions can be varied for both salt content and buffer content. For example the salt content can be less than 2M, 1.5M, 1M, 0.9M, 0.8M, 0.7M, 0.6M, 0.5M, 0.4M, 0.3M, 0.2M, 0.1M, 0.05M, or 0.02M. In certain embodiments the salt concentration is 500 mM or 150 mM.

There is no requirement for the particular salt. The salt can be Mg⁺² Mn⁺² Na⁺, K⁺ or other common mono-, di, or trivalent salts.

The methods can be performed at a variety of pH levels. For example, the methods can be performed at pH levels less than 10, 9, 8, 7, 6, 5 or greater than 5, 6, 7, 8, 9, 10 or between about 5 and 10 or about 6 and 9 or 6 and 8. In certain embodiments the pH level is about 9 or about 8 or about 7 or about 6 or about 5. Preferred pH levels are 8.0 and 7.2.

The methods can be performed at a variety of temperatures. For example the methods can be performed at temperatures ranging from 4-40° C. Typically, the methods will be performed at for example, less than 35° C. or 30° C. or 25° C. or 20° C. or 15° C. or 10° C. or 9° C. or 8° C. or 7° C. or 6° C. or 5° C. or 4° C.

In some embodiments the mixture further comprises 500 mM NaCl, and 50 mM Tris-HCl.

c) Inhibition Determination

For methods related to the modification of a particular amino acid contained in the CA protein, such that the modified amino acid or surrounding amino acids can be modified in a chemical or enzymatic way, the extent of chemical or enzymatic manipulation of the modified amino acid or CA protein containing the modified amino acid can be observed in any capable way. For example, if a chemical reaction takes place at the modified amino acid if the modified amino acid is accessible, the reaction can be monitored through, for example, radioactivity, fluorography, or any other detection means. If the modified amino acid can be involved in a protease reaction that takes place if the modified amino acid or surrounding amino acids are accessible, this proteolytic reaction can be monitored, by the release of radiolabel or fluor labeled peptide product.

In certain methods, the CA proteins assemble into cylindrical shapes, or the CA proteins assemble into conical shapes or the CA proteins assemble into a mixture of conical and cylindrical shapes based on whether the ˜600 Å³ cavity is occupied. This assembly can be monitored in any way that allows one to determine whether the conical or cylindrical shapes have assembled. Other ways to determine conical or cylindrical formation is through the use of transmission electron microscopy (TEM). It is preferred when using TEM that negatively stained samples are used. Formation can also be monitored by measuring light scattering at 312 nm (Abs₃₁₂=0.3-0.4 with a pathlength of 1 cm). When the assembly is monitored through light scattering, the reduction of assembly will register as a reduction in the amount of light scattering. Thus, molecules inhibit assembly will reduce the amount of light scattering in the light scattering measurement. Assays that use measure light scattering to determine the extent of cone formation can be performed under any conditions that allow the cone formation to be monitored. For example, the light scattering methods can be measured at different wavelengths from for example, 300 nm to 400 nm. Preferred wavelengths to are between 300 and 330 or 305 and 320 or 305 and 315 or 306 and 314 or 307 and 313 or 308 and 312 or 309 and 311. Preferred wavelengths are 309, 310, 311, 312, 313, 315, or 316. It is important that regardless of the wavelength the assay is performed at, the signal to noise ratios are low enough that formed structures can be detected. Pathlengths can be from 0.05 nm to 2 cm, but are preferred to be 1 cm.

Disclosed are methods of screening for molecules that inhibit HIV-1 capsid maturation comprising incubating a set of molecules with HIV-1 modified capsid proteins as disclosed herein, forming a molecule-capsid protein mixture, determining whether the capsid proteins have an immature conformation in vitro, by determining derivatization of an helix VI amino acid, for example, with increased derivatization indicating occupation of ˜600 Å³ cavity in the construct set forth in, for example, SEQ ID NO:3 or 5 or 12, and enriching the molecules that inhibit derivatization of a helix VI amino acid.

It is preferred that screens for inhibitors for screens have the capability to be high through put screens such as a batch assay or the use of a 96 or 384 well microtiter plate.

As discussed above, chemical libraries are well known in the art and any library may be used which may contain molecules that occupy ˜600 Å³ cavity of the CA protein.

2. CA-NC Capsid Assembly Assays

Disclosed are methods for testing a molecule for the potential to inhibit HIV-1 capsid formation comprising incubating the molecule with HIV-1 CA-NC protein together with a nucleic acid scaffold, and determining whether the molecule inhibits CA-NC assembly in vitro. For example, in certain embodiments the HIV-1 CA-NC protein can be produced using any recombinant biotechnology or synthetic method.

Disclosed are screening assays for isolating inhibitors of capsid assembly. Typically the screening assay would be performed as a high-throughput, batch assay in which a chemical library would be screened (e.g., in a 384 well plate format) and light scattering can be used to monitor CA-NC/DNA assembly. For example, the CA-NC(G94D) protein can be mixed together with the d(TG)₅₀ oligonucleotides, and compounds from the chemical library can be added into the reaction. The reaction mixture can be incubated overnight at 4° C., and light scattering at 312 nm measured for each reaction. Inhibitors of CA-NC/DNA assembly will reduce the light scattering by reducing the cylinder formation, and thereby score as “positives” in the assay.

a) CA-NC Protein

In some embodiments the CA-NC protein comprises amino acids 133-433 of the HIV gag protein (denoted CA-NC). A preferred form of the CA-NC protein are forms derived from the HV-1 strain NLA-3. However, there are a very large number of HV-1 strain variants, as discussed above, which can be found at for example the Los Alamos database which also produce CA-NC proteins that function in the disclosed methods.

There are known specific mutations in CA that block capsid assembly in the virus and these mutations also block CA-NC assembly in the disclosed assay.

In some embodiments, the gag protein contains a mutation of G to D at position 94 of SEQ ID NO:18 or a conserved variant thereof. It is understood that this G to D mutation can take place in any HIV-1 strain and that while the absolute position of the this variant may not stay the same in all strains, one of skill in the art understands which G corresponds to G94 of SEQ ID NO:18.

In certain embodiments the amino acids have the sequence set forth in SEQ ID NO:19 or a conserved variant thereof.

As with the CA-CTD domains set forth in SEQ ID NOS:15, 16, 17, and 25 and the representative nucleic acids that encode them set forth in SEQ ID NOS:23, 24, 26, and 27, the nucleic acid sequences encoding the CA-NC polypeptides disclosed herein are also described and disclosed, including all degenerate sequences. The nucleic acids encoding the disclosed and described variants of the CA-NC protein including SEQ ID NOS: 19-21 are also disclosed including all degenerate sequences.

b) Nucleic Acid Scaffold

The disclosed methods require a nucleic acid scaffold. This nucleic acid scaffold can be any template that promotes cylinder or conical formation. The nucleic acid scaffold may be comprised of native HIV nucleic acid or recombinant HIV nucleic acid. If the nucleic acid scaffold is HIV related, it may be any length that promotes formation of the cylinder or conical structure formation. In some embodiments the nucleic acid scaffold is less than 15,000 or 14,000 or 13,000 or 12,000 or 11,000 or 10,000 or 9,000 or 8,000 or 7,000 or 6,000 or 5,000 or 4,000 or 3,000 or 2000 or 1900 or 1800 or 1700 or 1600 or 1500 or 1400 or 1300 or 1200 or 1100 or 1000 or 900 or 800 or 700 or 600 or 500 or 400 or 300 or 200 or 100 nucleotides long.

For example, a nucleic acid such as the 6400-nt RNA from tobacco mosaic virus (TMV) functions in the disclosed methods as well as a 1400-nt fragment from the Bacillus stearothermophilus 16S ribosomal RNA. Thus, nucleic acid requirements are neither sequence specific or size specific. The symmetry of the cone produced in these methods is not specific for the viral RNA sequences or structures (for example, the DIS structure) available.

In general, any RNA or DNA sequence will work, but in order to run the assay at low concentrations of nucleic acid for example, between 1 and 10 uM, it should have stretches of alternating GT (or GU) residues.

Random sequences also function as the nucleic acid scaffold. This is indicated by the ability to use nucleic acid scaffolds obtained from different organisms but also from the fact that random sequence can be used as a nucleic acid scaffold also. For example, the lOOmer random sequence set forth in SEQ ID NO:22. While there are no particular sequence requirements to the nucleic acid some sequences are preferred. Certain embodiments preferably comprise a poly d(TG) sequence. The nucleic acid can comprise any number of d(TG) units. In certain embodiments, the nucleic acid comprises 300 or 250 or 200 or 150 or 100 or 90 or 80 or 70 or 60 or 50 or 45 or 40 or 35 or 30 or 25 or 20 or 15 or 10 or 5 d(TG) units. Preferred embodiments may have 50 or 38 or 25 units. In a preferred embodiment the nucleic acid scaffold has the sequence set forth in SEQ ID NO:19.

In certain embodiments the presence of the nucleic acid scaffold is not required. As the amount and or length of template is decrease, the salt requirements increase. For example when no nucleic acid scaffold is used in the disclosed methods the salt concentration should be at least 1 M, and 1 M NaCl is preferred.

### c) Reaction Conditions

Any reaction conditions that allow CA-NC assembly in the absence of an inhibitor or test molecule can be used for the disclosed assay. For example, the reaction conditions can be varied for both salt content and buffer content. For example the salt content can be less than 2M, 1.5M, 1M, 0.9M, 0.8M, 0.7M, 0.6M, 0.5M, 0.4M, 0.3M, 0.2M, 0.1M, 0.05M, or 0.02M. In certain embodiments the salt concentration is 500 mM or 150 mM.

There is no requirement for the particular salt. The salt can be Mg⁺² Mn⁺² Na⁺, K⁺ or other common mono-, di, or trivalent salts.

The methods can be performed at a variety of pH levels. For example, the methods can be performed at pH levels less than 10, 9, 8, 7, 6, 5 or greater than 5, 6, 7, 8, 9, 10 or between about 5 and 10 or about 6 and 9 or 6 and 8. In certain embodiments the pH level is about 9 or about 8 or about 7 or about 6 or about 5. Preferred pH levels are 8.0 and 7.2.

The methods can be performed at a variety of temperatures. For example the methods can be performed at temperatures ranging from 4-40° C. Typically, the methods will be performed at for example, less than 35° C. or 30° C. or 25° C. or 20° C. or 15° C. or 10° C. or 9° C. or 8° C. or 7° C. or 6° C. or 5° C. or 4° C. A preferred temperature to perform the methods at 4° C.

In some embodiments the mixture further comprises 500 mM NaCl, and 50 mM Tris-HCl.

In certain embodiments the mixture comprises 9 μM capsid protein and 1 μM d(TG)₅₀.

d) Inhibition Determination

In certain methods, the CA-NC proteins assemble into cylindrical shapes, or the CA-NC proteins assemble into conical shapes or the CA-NC proteins assemble into a mixture of conical and cylindrical shapes.

This assembly can be monitored in any way that allows one to determine whether the conical or cylindrical shapes have assembled.

Other ways to determine conical or cylindrical formation is through the use of transmission electron microscopy (TEM). It is preferred when using TEM that negatively stained samples are used.

Formation can also be monitored by measuring light scattering at 312 nm (Abs₃₁₂=0.3-0.4 with a pathlength of 1 cm). When the assembly is monitored through light scattering, the reduction of assembly will register as a reduction in the amount of light scattering. Thus, molecules inhibit assembly will reduce the amount of light scattering in the light scattering measurement.

Assays that measure light scattering to determine the extent of cone formation can be performed under any conditions that allow the cone formation to be monitored. For example, the light scattering experiments can be measured at different wavelengths from for example, 300 nm to 400 nm. Preferred wavelengths to are between 300 and 330 or 305 and 320 or 305 and 315 or 306 and 314 or 307 and 313 or 308 and 312 or 309 and 311. Preferred wavelengths are 309, 310, 311, 312, 313, 315, or 316. It is important that regardless of the wavelength the assay is performed at, the signal to noise ratios are low enough that formed structures can be detected. Pathlengths can be from 0.05 nm to 2 cm, but are preferred to be 1 cm.

3. Screening Methods for Inhibitors of CA-NC Assembly

Disclosed are methods of screening for molecules that inhibit of HIV-1 capsid formation comprising incubating a set of molecules with HIV-1 CA proteins forming a molecule-CA protein mixture, determining whether the CA proteins assemble in vitro, and enriching the molecules that inhibit capsid formation.

4. CA Dimerization Assay

a) Dimerization of CA

Disclosed are compositions and methods for performing a CA dimerization assay. The CA protein is capable of forming dimers and the formation of these dimers is required for core assembly and the assembly of infectious viral particles.

The HIV-1 CA protein comprises two domains separated by a flexible linker sequence. The N-terminal domain is essential for capsid formation, whereas the C-terminal dimerization domain is essential for forming both the immature particle and the mature capsid (Dorfman et al. J Virol. 68:8180-8187 (1994). High-resolution structures of both domains have been determined Gitti, R. K. et al. Science 273, 231-235 (1996); Momany, C. et al. Nature Struct. Biol. 3:763-770 (1996); Gamble, T. R. et al. Cell 87:1285-1294 (1996); Berthet-Colominas, C. et al. EMBO J 18:1124-1136 (1999); and Worthylake, D. K., Wang, H. Yoo, S., Sundquist, W. I. & Hill, C. P. Acta Crystallogr. D55:85-92 (1999) allowing molecular modeling of the reconstructed CA helix. A proposed model for the structure of the core is presented in Li et al., Nature 407:409-413 (2000) which is herein incorporated by reference for material related to the structure of the viral core.

Mutations that block dimerization also block viral replication. For example, changing Trp184 or Met185 to Ala results in loss of both dimerization and in viral replication.

Unique variations of the CA carboxy terminal domain (CTD) are disclosed which are capable of dimerizing with a higher affinity than the native CA CTD. The dimerization affinity of the native CA-CTD is inherently weak having a K_{d} for dimerization of approximately 20 μM. This low affinity makes it difficult to use the native CA protein in dimerization inhibitor screens because the typical inhibitor screen isolates molecules at or near the K_{d} of the competitive inhibitor in the assay, which in the case of a CA dimerization screen would typically be the CA dimerization domain.

Disclosed are compositions and methods for lowering the K_{d} of dimerization of the CA-CTD which increases the effectiveness of any competitive inhibitor screen. The disclosed compositions link two CA-CTD domains in tandem. This composition greatly lowers the K_{d} of dimerization. Tandem CA-CTD molecules have K_{d}s for another CA molecule which are typically less than 20 μM, 10 μM, 5 μM, 1 μM, 500 nM, 100 nM, 50 nM, 10 nM, 5 nM, 1 mM, 0.5 nM, 0.1 nM, 0.05 nM,or 0.01 nM.

The dimerization assay in one embodiment comprises: mixing various concentrations of CA-CTDs or derivatives of CA-CTDs together and determining whether dimerization has occurred. This assay can be used to test a variety of conditions related to dimerization, such as ionic requirements or nucleic acid requirements for dimerization.

A preferred form of the dimerization assay includes the step of determining dimerization formation through analysis of light scattering.

A scintillation proximity assay can also be used to determine whether dimer formation has occurred.

b) Screening for Inhibitors of Dimerization

Also disclosed are compositions and methods for using a CA dimerization assay to screen for inhibitors of dimerization. It is preferred that screens for inhibitors for screens have the capability to be high through put screens such as a batch assay or the use a 96 or 384 well microtiter plate.

The disclosed screening assay can use a scintillation proximity assay (SPA) to detect molecules that interfere with CA dimerization.

The screening assay for example can comprise mixing a library of molecules with native CA-CTD in a reaction mixture could comprising: 1) anti-FLAG antibody-derivatized SPA beads, 2) (CA-CTD))₂-FLAG protein, 3) ³H-(CA-CTD)₂, 4) compounds from the chemical library. ³H-(CA-CTD)₂/(CA-CTD)₂-FLAG complex formation via dimerization will bring ³H into close proximity to the scintillant and give rise to a light signal. Inhibitors of CA dimerization will be detected via reduction of this signal.

Disclosed is a method of screening for molecules that inhibit HIV-1 CA carboxy terminal domain dimerization comprising interacting a target molecule with a HIV-1 CA carboxy terminal domain forming a molecule-HIV-1 CA mixture and then interacting the mixture with a composition comprising a modified dimer of the HIV- CA CID, wherein the dimer is more stable than the dimer naturally.

Also disclosed is a method of screening for molecules that inhibit HIV-1 CA carboxy terminal domain dimerization comprising (a) interacting a target molecule with a HIV-1 CA carboxy terminal domain forming a molecule-HIV-1 CA mixture, (b) removing unbound molecules, (c) interacting the mixture with a modified CA carboxy terminal domain dimer as disclosed herein, and (d) collecting the molecules that interact with a composition comprising a modified dimer of the HIV-1 CA CTD, wherein the dimer is more stable than the dimer naturally forming a collection of HIV-1 CA carboxy terminal domain molecules.

Also disclosed are methods further comprising the step of repeating steps a-d above with the collection of carboxy terminal domain molecules.

Also disclosed is a method of screening for molecules that inhibit HIV-1 CA carboxy terminal domain dimerization comprising forming a modified dimer of the HIV-1 CA CTD, wherein the dimer is more stable than the dimer naturally making a dimer solution, interacting a target molecule with the dimer solution, and determining the dimer content of the dimer solution.

As discussed above, chemical libraries are well known in the art and any library may be used which may contain molecules that inhibit dimerization of the CA-CTD).

D. Sequences

Amino acid is numbered for Gag, that is, MA contains residues 1 to 132, and CA contains residues 133 to 363.

InDNASequence: R=A or G; Y=C or T; M=A or C; K=G or T; S=C or G; W=A or T; H=A or C or T; B=C or G or T; V=A or C or G; D=A or G or T; N=A or C or G or T.

```
1. SEQ ID NO:1 CA₃₆₃ (full-length CA) Protein
Sequence
        PIVQNLQGQMVHQAISPRTLNAWVKVVEEKAFSPEVIPMFSALSE
GATPQDLNTMLNTVGGHQAAMQMLKETINEEAAEWDRLHPVHAGPIAPGQ
MREPRGSDIAGTTSTLQEQIGWMTHNPPIPVGEIYKRWIILGLNKIVRMY
SPTSILDIRQGPKEPFRDYVDRFYKTLRAEQASQEVKNWMTETLLVQNAN
PDCKTILKALGPGATLEEMMTACQGVGGPGHKARVL
2. SEQ ID NO:2: DNA Sequence for full length CA
        CCNATHGTNCARAAYYTNCARGGNCARATGGTNCAYCARGCNATHW
SNCCNMGNACNYTNAAYGCNTGGGTNAARGTNGTNGARGARAARGCNTTY
WSNCCNGARGTNATHCCNATGTTYWSNGCNYTNWSNGARGGNGCNACNCC
NCARGAYYTNAAYACNATGYTNAAYACNGTNGGNGGNCAYCARGCNGCNA
TGCARATGYTNAARGARACNATHAAYGARGARGCNGCNGARTGGGAYMGN
YTNCAYCCNGTNCAYGCNGGNCCNATHGCNCCNGGNCARATGMGNGARCC
NMGNGGNWSNGAYATHGCNGGNACNACNWSNACNYTNCARGARCARATHG
GNTGGATGACNCAYAAYCGNCCNATHCCNGTNGGNGARATHTAYAARMGN
TGGATHATHYTNGGNYTNAAYAARATHGTNMGNATGTAYWSNCCNACNWS
NATHYTNGAYATHMGNCARGGNCCNAARGARCCNTTYMGNGAYTAYGTNG
AYMGNTTYTAYAARACNYTNMGNGCNGARCARGCNWSNCARGARGTNAAR
AAYTGGATGACNGARACNYTNYTNGTNCARAAYGCNAAYCCNGAYTGYAA
RACNATHYTNAARGCNYTNGGNCCNGGNGCNACNYTNGARGARATGATGA
CNGCNTGYCARGGNGTNGGNGGNCCNGGNCAYAARGCNMGNGTNYTN
3. SEQ ID NO:3 ₁₂₉MA-CA₂₇₈ Protein Sequence
        SQNYPIVQNLQGQMVHQAISPRTLNAWVKVVEEKAFSPEVIPMFSA
LSEGATPQDLNTMLNTVGGHQAAMQMLKETINEEAAEWDRLHPVHAGPIA
PGQMEPRGSDIAGTTSTLQEQIGWMTHNPPIPVGEIYKRWIILGLNKIVR
MYS
4. SEQ ID NO:4 ₁₂₉MA-CA₂₇₈ DNA Sequence
        WSNCARAAYTAYCCNATHGTNCARAAYYTNCARGGNCARATGGTNC
AYCARGCNATHWSNCCNMGNACNYTNAAYGCNTGGGTNAARGTNGTNGAR
GARAARGCNTTYWSNCCNGARGTNATHCCNATGTTYWSNGCNYTNWSNGA
RGGNGCNACNCCNCARGAYYTNAAYACNATGYTNAAYACNGTNGGNGGNC
AYCARGCNGCNATGCARATGYTNAARGARACNATHAAYGARGARGCNGCN
GARTGGGAYMGNYTNCAYCCNGTNCAYGCNGGNCCNATHGCNCCNGGNCA
RATGMGNGARCCNMGNGGNWSNGAYATHGCNGGNACNACNWSNACNYTNC
ARGARCARATHGGNTGGATGACNCAYAAYCCNCCNATHCCNGTNGGNGAR
ATHTAYAARMGNTGGATHATHYTNGGNYTNAAYAARATHGTNMGNATGTA
YWSN
5. SEQ ID NO:5 ₁₀₅MA-CA₂₇₈ Protein Sequence
        EEEQNKSKKKAQQAAADTGNNSQVSQNYPIVQNLQGQMVHQAISPR
TLNAWVKVVEEKAFSPEVIPMFSALSEGATPQDLNTMLNTVGGHQAAMQM
LKETINEEAAEWDRLHPVHAGPIAPGQMREPRGSDIAGTTSTLQEQIGWM
THNPPIPVGEIYKRWIILGLNKIVRMYS
6. SEQ ID NO:6 ₁₀₅MA-CA₂₇₈ DNA Sequence
        GARGARGARCARAAYAARWSNAARAARAARGCNCARCARGCNGCNG
CNGAYACNGGNAAYAAYWSNCARGTNWSNCARAAYTAYCCNATHGTNCAR
AAYYTNCARGGNCARATGGTNCAYCARGCNATHWSNCCNMGNACNYTNAA
YGCNTGGGTNAARGTNGTNGARGARAARGCNTTYWSNCCNGARGTNATHC
CNATGTTYWSNGCNYTNWSNGARGGNGCNACNCCNCARGAYYTNAAYACN
ATGYTNAAYACNGTNGGNGGNCAYCARGCNGCNATGCARATGYTNAARGA
RACNATHAAYGARGARGCNGCNGARTGGGAYMGNYTNCAYCCNGTNCAYG
CNGGNCCNATHGCNCCNGGNCARATGMGNGARCCNMGNGGNWSNGAYATH
GCNGGNACNACNWSNACNYTNCARGARCARATHGGNTGGATGACNCAYAA
YCCNCCNATHCCNGTNGGNGARATHTAYAARMGNTGGATHATHYTNGGNY
TNAAYAARATHGTNMGNATGTAYWSN
7. SEQ ID NO:7 CA₃₆₄₋₁₂₉MA-CA₂₇₈ Protein Sequence I
to V at position 6
        SQNYPVVQNLQGQMVHQAISPRTLNAWVKVVEEKAFSPEVIPMFSA
LSEGATPQDLNTMLNTVGGHQAAMQMLKETINEEAAEWDRLHPVHAGPIA
PGQMREPRGSDIAGTTSTLQEQIGWMTHNPPIPVGEIYKRWIILGLNKIV
RMYS
8. SEQ ID NO:8: DNA Sequence encoding SEQ ID
NO:7
        WSNCARAAYTAYCCNGTNGTNCARAAYYTNCARGGNCARATGGTNC
AYCARGCNATHWSNCCNMGNACNYTNAAYGCNTGGGTNAARGTNGTNGAR
GARAARGCNTTYWSNCCNGARGTNATHCCNATGTTYWSNGCNYTNWSNGA
RGGNGCNACNCCNCARGAYYTNAAYACNATGYTNAAYACNGTNGGNGGNC
AYCARGCNGCNATGCARATGYTNAARGARACNATHAAYGARGARGCNGCN
GARTGGGAYMGNYTNCAYCCNGTNCAYGCNGGNCCNATHGCNCCNGGNCA
RATGMGNGARCCNMGNGGNWSNGAYATHGCNGGNACNACNWSNACNYTNC
ARGARCARATHGGNTGGATGACNCAYAAYCCNCCNATHCCNGTNGGNGAR
ATHTAYAARMGNTGGATHATHYTNGGNYTNAAYAARATHGTNMGNATGTA
YWSN
9. SEQ ID NO:9: Protein sequence for full length
MA-CA
        MGARASVLSGGELDKWEKIRLRPGGKKQYKLKHIVWASRRLERFAV
NPGLLETSEGCRQILGQLQPSLQTGSEELRSLYNTIAVLYCVHQRIDVKD
TKEALDKIEEEQNKSKKKAQQAAADTGNNSQVSQNYPIVQNLQGQMVHQA
ISPRTLNAWVKVVEEKAFSPEVIPMFSALSEGATPQDLNTMLNTVGGHQA
AMQMLKETINEEAAEWDRLHPVHAGPIAPGQMREPRGSDIAGTTSTLQEQ
IGWMTHNPPIPVGEIYKRWIILGLNKIVRMYSPTSILDIRQGPKEPFRDY
VDRFYKTLRAEQASQEVKNWMTETLLVQNANPDCKTILKALGPGATLEEM
MTACQGVGGPGHKARVL
10. SEQ ID NO:10; DNA sequence for full length MA-
CA CA
        ATGGGNGCNMGNGCNWSNGTNYTNWSNGGNGGNGARYTNGAYAART
GGGARAARATHMGNYTNMGNCCNGGNGGNAARAARCARTAYAARYTNAAR
CAYATHGTNTGGGCNWSNMGNGARYTNGARMGNTTYGCNGTNAAYCCNGG
NYTNYTNGARACNWSNGARGGNTGYMGNCARATHYTNGGNCARYTNCARC
CNWSNYTNCARACNGGNWSNGARGARYTNMGNWSNYTNTAYAAYACNATH
GCNGTNYTNTAYTGYGTNCAYCARMGNATHGAYGTNAARGAYACNAARGA
RGCNYTNGAYAARATHGARGARGARCARAAYAARWSNAARAARAARGCNC
ARCARGCNGCNGCNGAYACNGGNAAYAAYWSNCARGTNWSNCARAAYTAY
CCNATHGTNCARAAYYTNCARGGNCARATGGTNCAYCARGCNATHWSNCC
NMGNACNYTNAAYGGNTGGGTNAARGTNGTNGARGARAARGCNTTYWSNC
CNGARGTNATHCCNATGTTYWSNGCNYTNWSNGARGGNGCNACNCGNCAR
GAYYTNAAYACNATGYTNAAYACNGTNGGNGGNCAYCARGCNGCNATGCA
RATGYTNAARGARACNATHAAYGARGARGCNGCNGARTGGGAYMGNYTNC
AYCCNGTNCAYGCNGGNCCNATHGCNCCNGGNCARATGMGNGARCCNMGN
GGNWSNGAYATHGCNGGNACNACNWSNACNYTNCARGARCARATHGGNTG
GATGACNCAYAAYCCNCCNATHCCNGTNGGNGARATHTAYAARMGNTGGA
THATHYTNGGNYTNAAYAARATHGTNMGNATGTAYWSNCCNACNWSNATH
YTNGAYATHMGNCARGGNCCNAARGARCCNTTYMGNGAYTAYGTNGAYMG
NTTYTAYAARACNYTNMGNGCNGARCARGCNWSNCARGARGTNAARAAYT
GGATGACNGARACNYTNYTNGTNCARAAYGCNAAYCCNGAYTGYAARACN
ATHYTNAARGCNYTNGGNCCNGCNGCNACNYTNGARGARATGATGACNGC
NTGYCARGGNGTNGGNGGNCCNGGNCAYAARGCNMGNGTNYTN
11. SEQ ID NO: 11, sequence of conserved MHR
region in CA
        IRQGPKEPFRDYVDRFYKTL
12. SEQ ID NO:12 ₁₀₅MA-CA₂₇₈ with I247C mutation
Protein Sequence
        EEEQNKSKKKAQQAAADTGNNSQVSQNYPIVQNLQGQMVHQAISPR
TLNAWVKVVEEKAFSPEVIPMFSALSEGATPQDLNTMLNTVGGHQAAMQM
LKETINEEAAEWDRLHPVHAGPIAPGQMREPRGSDIAGTTSTLQEQCGWM
THNPPIPVGEIYKRWIILGLNKIVRMYS
13. SEQ ID NO:13 ₁₀₅MA-CA₂₇₈ with I247C mutation
DNA Sequence encoding SEQ ID NO:12
        GARGARGARCARAAYAARWSNAARAARAARGCNCARCARGCNGCNG
CNGAYACNGGNAAYAAYWSNCARGTNWSNCARAAYTAYCCNATHGTNCAR
AAYYTNCARGGNCARATGGTNCAYCARGCNATHWSNCCNMGNACNYTNAA
YGCNTGGGTNAARGTNGTNGARGARAARGCNTTYWSNCCNGARGTNATHC
CNATGTTYWSNGCNYTNWSNGARGGNGCNACNCCNCARGAYYTNAAYACN
ATGYTNTAAYACNGTNGGNGGNCAYCARGCNGCNATGCARATGYTNAARG
ARACNATHAAYGARGARGCNGCNGARTGGGAYMGNYTNCAYCCNGTNCAY
GCNGGNCCNATHGCNCCNGGNCARATGMGNGARCCNMGNGGNWSNGAYAT
HGCNGGNACNACNWSNACNYTNCARGARCARTGYGGNTGGATGACNCAYA
AYCCNCCNATHCCNGTNGGNGARATHTAYAARMGNTGGATHATHYTNGGN
YTNAAYAARATHGTNMGNATGTAYWSN
14. SEQ ID NO:14 ₁₂₉MA-CA₂₇₈ Protein Sequence with
a C at 247
        SQNYPIYQNLQGQMVHQAISPRTLNAWVKVVEEKAFSPEVIPMFSA
LSEGATPQDLNTMLNTVGGHQAAMQMLKETINEEAAEWDRLHPVHAGPIA
PGQMREPRGSDIAGTTSTLQEQCGWMTHNPPIPVGEIYKRWIILGLNKIV
RMYS
15. SEQ ID NO:15 example of a CA-CTD
        MSPTSILDIRQGPKEPFRDYVDRFYKTLRAEQASQEVKNWMTETLL
VQNANPDCKTILKALGPGATLEEMMTACQGVG
16. SEQ ID NO:16 example of a modified dimer (CA-
CTD)₂
        MSPTSILDIRQGPKEPFRDYVDRFYKTLRAEQASQEVKNWMTETLL
VQNANPDCKTILKALGPGATLEEMMTACQGVGPWSPTSILDIRQGPKEPF
RDYVDRFYKTLRAEQASQEVKNWMTETLLVQNANPDCKTILKALGPGATL
EEMMTACQGVG
17. SEQ ID NO:17 example of modified dimer with
flag sequence (CA-CTD)₂-FLAG
        MSPTSILDIRQGPKEPFRDYVDRFYKTLRAEQASQEVKNWMTETLL
VQNANPDCKTILKALGPGATLEEMMTACQGVGPWSPTSILDIRQGPKEPF
RDYVDRLFYKTLRAEQASQEVKNWMTETLLVQNANPDCKTILKALGPGAT
LEEMMTACQGVGGGDYKDDDDK
18. SEQ ID NO:18 an example of a wild type version
of CA-NC
        PIVQNLQGQMVHQAISPRTLNAWVKVVEEKAFSPEVIPMFSALSEG
ATPQDLNTMLNTVGGHQAAMQMLKETINEEAAEWDRLHPVHAGPIAPGQM
REPRGSDIAGTTSTLQEQIGWMTHNPPIPVGEIYKRWIILGLNKIVRMYS
PTSILDIRQGPKEPFRDYVDRFYKTLRAEQASQEVKNWMTETLLVQNANP
DCKTILKALGPGATLEEMMTACQGVGGPGHKARVLAEAMSQVTNPATIMI
QKGNFRNQRKTVKCFNCGKEGHIAKNCRAPRKKGCWKCGKEGHQMKDCTE
RQAN
19.SEQ ID NO:19 an example of a derivative CA-NC
(G94D)
        PIVQNLQGQMVHQAISPRTLNAWVKVVEEKAFSPEVIPMFSALSEG
ATPQDLNTMLNTVGGHQAAMQMLKETINEEAAEWDRLHPVHAGPIAPDQM
REPRGSDIAGTTSTLQEQIGWMTHNPPIPVGEIYKRWIILGLNKIVRMYS
PTSILDIRQGPKEPFRDYVDRFYKTLRAEQASQEVKNWMTETLLVQNANP
DCKTILKALGPAGTLEEMMTACQGVGGPGHKARVLAEAMSQVTNPATIMI
QKGNFRNQRKTVKCFNCGKEGHIAKNCRAPRKKGCWKCGKEGHQMKDCTE
RQAN
20. SEQ ID NO: 20 an example of a mutant CA-NC
protein that block assembly in vitro (G94D/A42D)
        PIVQNLQGQMVHQAISPRTLNAWVKVVEEKAFSPEVIPMFSDLSEG
ATPQDLNTMLNTVGGHQAAMQMLKETINEEAAEWDRLHPVHAGPIAPDQM
REPRGSDIAGTTSTLQEQIGWMTHNPPIPVGEIYKRWIILGLNKIVRMYS
PTSILDIRQGPKEPFRDYVDRFYKTLRAEQASQEVKNWMTETLLVQNANP
DCKTILKALGPGATLEEMMTACQGVGGPGHKARVLAEAMSQVTNPATIMI
QKGNFRNQRKTVKCFNCGKEGHIANCRAPRKKGCWKCGKEGHQMKDCTER
QAN
21. SEQ ID NO:21 an example of a mutant CA-NC
protein that blocks assembly in vitro
(G94D/W184A/M185A)
        PIVQNLQGQMVHQAISPRTLNAWVKVVEEKAFSPEVIPMYSAISEG
ATPQDLNTMLNTVGGHQAAMQMLKETINEEAAEWDRLHPVHAGPIAPDQM
REPRGSDIAGTTSTLQEQIGWMTHNPPIPVGEIYKRWIILGLNKIVRMYS
PTSILDIRQGPKEPFRDYVDRFYKTLRAEQASQEVKNAATETLLVQNANP
DCKTILKALGPGATLEEMMTACQGVGGPGHKARVLAEAMSQVTNPATIMI
QKGNFRNQRKTVKCFNCGKEGHIAKNCRAPRKKGCWKCGKEGHQMKDCTE
RQAN
22. SEQ ID NO:22 a flag sequence
        GGDYKDDDDK
23. SEQ ID NO:23 an example of nucleic acid
sequence that encodes SEQ ID NO15
        ATGAGCCCTACCAGCATTCTGGACATAAGACAAGGACCAAAGGAAC
CCTTTAGAGACTATGTAGACCGATTCTATAAAACTCTAAGAGCCGAGCAA
GCTTCACAAGAGGTAAAAAATTGGATGACAGAAACCTTGTTGGTCCAAAA
TGCGAACCCAGATTGTAAGACTATTTTAAAAGCATTGGGACCAGGAGCGA
CACTAGAAGAAATGATGACAGCATGTCAGGGAGTGGGG
24. SEQ ID NO:24, a generic sequence listing
showing all degenerate nucleic acid sequences
based on the third position of the codons that
encode SEQ ID NO:15
        ATGWSNCCNACNWSNATHYTNGAYATHMGNCARGGNCCNAARGARC
CNTTYMGNGAYTAYGTNGAYMGNTTYTAYAARACNYTNMGNGCNGARCAR
GCNWSNCARGARGTNAARAAYTGGATGACNGARACNYTNYTNGTNCARAA
YGCNAAYCCNGAYTGYAARACNATHYTNAARGCNYTNGGNCCNGGNGCNA
CNYTNGARGARATGATGACNGCNTGYCARGGNGTNGGN
25. SEQ ID NO:25 amino acid sequence of CA with
Ile6 to Val mutation in a CA-CTD
        MSPTSVLDIRQGPKEPFRDYVDRFYKTLRAEQASQEVKNWMTETLL
VQNANPDCKTILKALGPGATLEEMMTACQGVG
26. SEQ ID NO:26 nucleic acid sequence that encodes
SEQ ID NO:25 with Ile6 to Val mutation
        ATGAGCCCTACCAGCGTTCTGGACATAAGACAAGGACCAAAGGAAC
CCTTTAGAGACTATGTAGACCGATTCTATAAAACTCTAAGAGCCGAGCAA
GCTTCACAAGAGGTAAAAAATTGGATGACAGAAACCTTGTTGGTCCAAAA
TGCGAACCCAGATTGTAAGACTATTTTAAAAGCATTGGGACCAGGAGCGA
CACTAGAAGAAATGATGACAGCATGTCAGGGAGTGGGG
27. SEQ ID NO:27 degenerate nucleic acid sequences
that encodes SEQ ID NO25 with Ile6 to Val mutation
showing all degenerate nucleic acid sequences
based on the third position of the codons that
encode SEQ ID NO:25.
        ATGWSNCCNACNWSNGTNYTNGAYATHMGNCARGGNCCNAARGARC
CNTTYMGNGAYTAYGTNGAYMGNTTYTAYAARACNYTNMGNGCNGARCAR
GCNWSNCARGARGTNAARAAYTGGATGACNGARACNYTNYTNGTNCARAA
YGCNAAYCCNGAYTGYAARACNATHYTNAARGCNYTNGGNCCNGGNGCNA
CNYTNGARGARATGATGACNGCNTGYCARGGNGTNGGN
28. SEQ ID NO:28 oligonucleotide d(TG)n
        TGTGTGTGTG TGTGTGTGTG TGTGTGTGTG TGTGTGTGTG
TGTGTGTGTG
```

E. References
Butcher, S. J., Dokland, T., Ojala, P. M., Bamford, D. H., and Fuller, S. D. (1997). Intermediates in the assembly pathway of the double-stranded RNA virus phi6, Embo J 16, 4477-87.
Campbell, S., Fisher, R. J., Towler, E. M., Fox, S., Issaq, H. J., Wolfe, T., Phillips, L. R., and Rein, A. (2001). Modulation of HIV-like particle assembly in vitro by inositol phosphates, Proc Natl Acad Sci U S A 28, 28.
Campbell, S., and Rein, A. (1999). In vitro assembly properties of human immunodeficiency virus type 1 Gag protein lacking the p6 domain, J Virol 73, 2270-9.
Campbell, S., and Vogt, V. M. (1995). Self-assembly in vitro of purified CA-NC proteins from Rous sarcoma virus and human immunodeficiency vims type 1, J Virol 69, 6487-97.
Canady, M. A., Tihova, M., Hanzlik, T. N., Johnson, J. E., and Yeager, M. (2000). Large conformational changes in the maturation of a simple RNA virus, nudaurelia capensis omega virus (NomegaV), J Mol Biol 299, 573-84.
Chow, M., Basavappa, R., and Hogle, J. M. (1997). The role of conformational transitions in poliovirus pathogenesis, In Structural Biology of Viruses, 157-86. Oxford University Press, New York.
Conway, J. F., Wikoff, W. R., Cheng, N., Duda, R. L., Hendrix, R. W., Johnson, J. E., and Steven, A. C. (2001). Virus maturation involving large subunit rotations and local refolding, Science 292, 744-8.
Duda, R. L., Hempel, J., Michel, H., Shabanowitz, J., Hunt, D., and Hendrix, R. W. (1995). Structural transitions during bacteriophage HK97 head assembly, J Mol Biol 247, 618-35.
Ellman, G. L. (1959). Tissue sulfhydryl groups, Arch Biochem Biophys 82, 70-77.
Erickson-Viitanen, S., Manfredi, J., Viitanen, P., Tribe, D. B., Tritch, R., Hutchison, C. A., 3rd, Loeb, D. D., and Swanstrom, R. (1989). Cleavage of HIV-1 gag polyprotein synthesized in vitro: sequential cleavage by the viral protease, AIDS Res Hum Retroviruses 5, 577-91.
Gamble, T. R., Vajdos, F. F., Yoo, S., Worthylake, D. K., Houseweart, M., Sundquist, W. I., and Hill, C. P. (1996). Crystal structure of human cyclophilin A bound to the amino-terminal domain of HIV-1 capsid, Cell 87, 1285-94.
Ganser, B. K., Li, S., Klishko, V. Y., Finch, J. T., and Sundquist, W. I. (1999). Assembly and analysis of conical models for the HIV-1 core, Science 283, 80-3.
Gitti, R. K., Lee, B. M., Walker, J., Summers, M. F., Yoo, S., and Sundquist, W. I. (1996). Structure of the amino-terminal core domain of the HIV-1 capsid protein, Science 273, 231-5.
Gottlinger, H. G., Sodroski, J. G., and Haseltine, W. A. (1989). Role of capsid precursor processing and myristoylation in morphogenesis and infectivity of human immunodeficiency virus type 1, Proc Natl Acad Sci U S A 86,5781-5.
Gross, I., Hohenberg, H., Huckhagel, C., and Krausslich, H. G. (1998). N-Terminal extension of human immunodeficiency virus capsid protein converts the in vitro assembly phenotype from tubular to spherical particles, J Virol 72, 4798-810.
Gross, I., Hohenberg, H., and Krausslich, H. G. (1997). In vitro assembly properties of purified bacterially expressed capsid proteins of human immunodeficiency virus, Eur J Biochem 249, 592-600.
Gross, I., Hohenberg, H., Wilk, T., Wiegers, K., Grattinger, M., Muller, B., Fuller, S., and Krausslich, H. G. (2000). A conformational switch controlling HIV-1 morphogenesis, Embo J 19, 103-13.
Johnson, J. E. (1996). Functional implications of protein-protein interactions in icosahedral viruses, Proc Natl Acad Sci U S A 93, 27-33.
Johnson, J. E.,andSpeir,J. A. (1997). Quasi-equivalentviruses:a paradigm for protein assemblies, J Mol Biol 269, 665-75.
Konvalinka, J., Heuser, A. M., Hruskova-Heidingsfeldova, O., Vogt, V. M., Sedlacek, J., Strop, P., and Krausslich, H. G. (1995). Proteolytic processing of particle-associated retroviral polyproteins by homologous and heterologous viral proteinases, Eur J Biochem 228, 191-8.
Krausslich, H. G. (1996). Morphogenesis and maturation of retroviruses, Current Topics in Immunology, Springer-verlag, Berlin.
Krausslich, H. G., Schneider, H., Zybarth, G., Carter, C. A., and Wimmer, E. (1988). Processing of in vitro-synthesized gag precursor proteins of human immunodeficiency virus (HIV) type 1 by HIV proteinase generated in Escherichia coli, J Virol 62, 4393-7.
Kunkel, T. A., Roberts, J. D., and Zakour, R. A. (1987). Rapid and efficient site-specific mutagenesis without phenotypic selection, Methods Enzymol 154, 367-82.
Li, S., Hill, C. P., Sundquist, W. I., and Finch, J. T. (2000). Image reconstructions of helical assemblies of the HIV-1 CA protein, Nature 407, 409-13.
Pettit, S. C., Moody, M. D., Wehbie, R. S., Kaplan, A. H., Nantermet, P. V., Klein, C. A., and Swanstrom, R. (1994). The p2 domain of human immunodeficiency virus type 1 Gag regulates sequential proteolytic processing and is required to produce fully infectious virions, J Virol 68, 8017-27.
Stemmler, T. L., Alam, S. L., Wang, H., Davis, D. R., and Sundquist, W. I. (2001).
Tritch, R. J., Cheng, Y. E., Yin, F. H., and Erickson-Viitanen, S. (1991). Mutagenesis of protease cleavage sites in the human immunodeficiency virus type 1 gag polyprotein, J Virol 65, 922-30.
Trus, B. L., Booy, F. P., Newcomb, W. W., Brown, J. C., Homa, F. L., Thomsen, D. R., and Steven, A. C. (1996). The herpes simplex virus procapsid: structure, conformational changes upon maturation, and roles of the triplex proteins VP19c and VP23 in assembly, J Mol Biol 263,447-62.
Turner, B. G., and Summers, M. F. (1999). Structural biology of HIV, J Mol Biol 285, 1-32.
von Schwedler, U. K., Stemmler, T. L., Klishko, V. Y., Li, S., Albertine, K. H., Davis, D. R., and Sundquist, W. I. (1998). Proteolytic refolding of the HIV-1 capsid protein amino-terminus facilitates viral core assembly, Embo J 17, 1555-68.
Wiegers, K., Rutter, G., Kottler, H., Tessmer, U., Hohenberg, H., and Krausslich, H. G. (1998). Sequential steps in human immunodeficiency virus particle maturation revealed by alterations of individual Gag polyprotein cleavage sites, J Virol 72, 2846-54.
Barldey, A, Arya P, “Combinatorial chemistry toward understanding the function(s) of carbohydrates and carbohydrate conjugates, Chemistry. 2001 Feb. 2;7(3):555-63.
Berthet-Colominas, C. et al. “Head-to-tail dimers and interdomain flexibility revealed by the crystal structure of HIV-1 capsid protein (;24) complexed with amonoclonal antibody Fab.,” EMBO J 18:1124-1136 (1999).
Bohm, H. J., Stahl, M., “Structure-based library design: molecular modelling merges with combinatorial chemistry,” Curr Opin Chem Biol. 2000 June ;4(3):283-6.
Chauhan PM, Srivastava SK, “Recent developments in the combinatorial synthesis of nitrogen heterocycles using solid technology,” Comb Chem High Throughput Screen. 2001 February ;4(1):35-51.
Curran, D P, Josien H, Bom D, Gabarda A E, Du W, “The cascade radical annulation approach to new analogues of camptothecins,” Combinatorial synthesis of silatecans and homosilatecans, Ann N Y Acad Sci. 2000;922: 112-21. 21.
de Julian-Ortiz J V, “Virtual Darwinian drug design: QSAR inverse problem, virtual combinatorial chemistry, and computational screening,” Comb Chem High Throughput Screen. 2001 May ;4(3):295-310.
Dorfinan et al. “Functional domains of the capsid protein of human immunodeficiency virus type 1,” J. Virol. 68:8180-8187 (1994).
Fisher, R. J., Rein, A., Fivash, M., Urbaneja, M. A., Casas-Finet, J. R., Medaglia, M., and Henderson, L. E. 1998. Sequence-specific binding of human immunodeficiency virus type 1 nucleocapsid protein to short oligonucleotides. J Virol., 72:1902-1909.
Floyd, C. D., Leblanc C, Whittaker M., “Combinatorial chemistry as a tool for drug discovery,” Prog Med Chem. 1999;36:91-168; 45:
Furka A, Bennett WD, “Combinatorial libraries by portioning and mixing,” Comb Chem High Throughput Screen. 1999 April ;2(2):105-22.
Gamble, T. R., Vajdos, F. F., Yoo, S., Worthylake, D. K., Houseweart, M., Sundquist, W. I. and Hill, C. P. 1996. Cell. 87:1285-1294.
Gamble, T. R., Yoo, S., Vajdos, F. F., von Schwedler, U. K., Worthylake, D. K., Wang, H., McCutcheon, J. P., Sundquist, W. I., and Hill, C. P. 1997. Science. 278:849-853.
Ganser, B. K., Li, S., Klishko, V. Y., Finch, J. T. Sundquist, W. I. “Assembly and Analysis of Conical Models for the HIV-1 Core,” 1999. Science 283:80-83.
Gitti, R. K et al. “Structure of the amino-terminal core domain of the HIV-1 capsid protein,” Science 273, 231-235 (1996).
Hermanson, C. T. et al., eds.,Inmobilized Affinity Ligands, (Academic Press, New York, 1992).
Houghten, R A., “Parallel array and mixture-based synthetic combinatorial chemistry: tools for the next millennium, “Annu Rev Pharmacol Toxicol. 2000; 40:273-82.
Huc, I., Nguyen, R. “Dynamic combinatorial chemistry, Comb Chem High Throughput Screen. 2001 February ;4(1):53-74.
Johnstone and Thorpe, Immunochemistry In Practice (Blackwell Scientific Publications, Oxford, England, 1987) pages 209-216 and 241-242.
Kerkhof, 1992 Anal. Biochem. 205:359-364.
Kirlpatrick, D L, Watson S, Ulhaq S., “Structure-based drug design: combinatorial chemistry and molecular modeling,” Comb Chem High Throughput Screen. 1999 August ;2(4):211-21.
Krausslich, H-G Ed. Morphogenesis and Maturation of Retroviuruses Vol 214 Current Trends in Microbiology and Immunology (Springer-Verlag, Berlin 1996).
Kunkel, T. A., Roberts, J. D., and Zakour, R. A. 1987. Rapid and efficient site-specific mutagenesis without phenotypic selection. Methods Enzymol., 154:367-382.
Li, S., Hill, C. P., Sundquist, W. I., and Finch, J. T. 2000. Image reconstructions of helical assemblies of the HIV-1 CA protein. Nature, 407:409-413.
Momany, C. et al. “Crystal structure of dirneric HIV-1 capsid protein,”. Nature Struct. Biol. 3:763-770 (1996).
Nestler, H. P. , Liu R., “Combinatorial libraries: studies in molecular recognition,” Comb Chem High Throughput Screen. 1998 October ;1(3):113-26; 48.
Oliver S F, Abell C., “Combinatorial synthetic design,” Cur Opin Chem Biol. 1999 June;3(3):299-306.
Schweizer F, Hindsgaul O., “Combinatorial synthesis of carbohydrates,” Curr Opin Chem Biol. 1999 June;3(3):291-8.
Swanstrom, R. and Willis J. W. in Retroviruses J. M. Coffin S. H. Hughes, and H. E. Varmus Eds. (Cold Spring harbor Laboratory press Plainview NY, 1997 pp 263-334.
Taylor, Richard F. ed. (M. Dekker, New Yorlc, 1991).
U.S. Pat. No. 6,255,120 for “Combinatorial library of substituted statine esters and amides via a novel acid-catalyzed Rearrangement;”
U.S. Pat. No. 6,207,820 for “Combinatorial library of moenomycin analogs and methods of producing same;”
U.S. Pat. No. 6,168,912 for “Method and kit for making a multidimensional combinatorial chemical library;”
U.S. Pat. No. 6,114,309 for “Combinatorial library of moenomycin analogs and methods of producing same;”
U.S. Pat. No. 6,025,371 for “Solid phase and combinatorial library syntheses of fused 2,4-pyrimidinediones;”
U.S. Pat. No. 6,017,768 for Combinatorial dihydrobenzopyran library;” U.S. Pat. No. 5,962,337 for Combinatorial 1,4-benzodiazepin-2,5-dione library;”
U.S. Pat. No. 5,919,955 for “Combinatorial solid phase synthesis of a library of benzofuran derivatives;”
U.S. Pat. No. 5,856,496 for “Combinatorial solid phase synthesis of a library of indole derivatives;”
U.S. Pat. No. 5,821,130 for “Combinatorial dihydrobenzopyran library;”
U.S. Pat. No. 5,712,146 for “Recombinant combinatorial genetic library for the production of novel polyketides;”
U.S. Pat. No. 5,698,685 for “Morpholino-subunit combinatorial library and method;”
U.S. Pat. No. 5,688,997 for “Process for preparing intermediates for a combinatorial dihydrobenzopyran library;”
U.S. Pat. No. 5,618,825 for Combinatorial sulfonamide library” von Schwedler, Uta. K., Stemmler, T. L., Klishko, V. Y., Li, S., Albertine, K., Davis, D. R., and Sundquist, W. I. 1997. EMBO J., 17(6):1555-1568.
Weber, L., “Hgh-diversity combinatorial libraries, Curr Opin Chem Biol. 2000 June;4(3):295-302.
Worthylake, D. K., Wang, H. Yoo, S., Sundquiest, W. I. & Hill, C. P. “Structures of the HIV- 1 capsid protein dimerization domain at 2.6A resolution,” Acta Crystallogr. D55:85-92 (1999).
Worthylake, D. K., Wang, H., Yoo, S., Sundquist, W. I., Hill, C. P. 1998. Structures of the HV-1 capsid protein dimerization domain at 2.6A resolution Biological Crystallography D55:85-92.
1. Krausslich, H. G. (ed.) Morphogenesis and Maturation of Retroviruses, (Springer-Verlag, Berlin, 1996).
2. Fu, W., Gorelick, R. J. & Rein, A. Characterization of human immunodeficiency virus type 1 dimeric RNA from wild-type and protease-defective virions. J Virol 68, 5013 (1994).
3. Gelderblom, H. R., Ozel, M. & Pauli, G. Morphogenesis and morphology of HIV. Structure-function relations. Arch Virol 106, 1 (1989).
4. Turner, B. G. & Summers, M. F. Structural biology of HIV. J Mol Biol 285, 1 (1999).
5. Facke, M., Janetzko, A., Shoeman, R. L. & Krausslich, H.G. A large deletion in the matrix domain of the human immunodeficiency virus gag gene redirects virus particle assembly from the plasma membrane to the endoplasmic reticulum. J Virol 67, 4972 (1993).
6. Yuan, X., Yu, X., Lee, T. H. & Essex, M. Mutations in the N-terminal region of human immunodeficiency virus type 1 matrix protein block intracellular transport of the Gag precursor. J Virol 67, 6387 (1993).
7. Freed, E. O., Orenstein, J. M., Buckler-White, A. J. & Martin, M. A. Single amino acid changes in the human immunodeficiency virus type 1 matrix protein block virus particle production. J Virol 68, 5311 (1994).
8. Freed, E. O., Englund, G. & Martin, M. A. Role of the basic domain of human immunodeficiency virus type 1 matrix in macrophage infection. J Virol 69, 3949 (1995).
9. Zhou, W. & Resh, M. D. Differential membrane binding of the human immunodeficiency virus type 1 matrix protein. J Virol 70, 8540 (1996).
10. Cannon, P. M. et al. Structure-function studies of the human immunodeficiency virus type 1 matrix protein, p17. J Virol 71, 3474 (1997).
11. Spearman, P., Horton, R., Ratner, L. & Kuli-Zade, I. Membrane binding of human immunodeficiency virus type 1 matrix protein in vivo supports a conformational myristyl switch mechanism. J Virol 71, 6582 (1997).
12. Paillart, J. C. & Gottlinger, H. G. Opposing effects of human immunodeficiency virus type 1 matrix mutations support a myristyl switch model of gag membrane targeting. J Virol 73, 2604 (1999).
13. Ono, A. & Freed, E. O. Binding of human immunodeficiency virus type 1 Gag to membrane: role of the matrix amino terminus. J Virol 73, 4136 (1999).
14. Morikawa, Y., Hockley, D. J., Nermut, M. V. & Jones, I. M. Roles of matrix, p2, and N-terminal myristoylation in human immunodeficiency virus type 1 Gag assembly. J Virol 74, 16 (2000).
15. Ono, A., Orenstein, J. M. & Freed, E. O. Role of the Gag matrix domain in targeting human immunodeficiency virus type 1 assembly. J Virol 74, 2855 (2000).
16. Dorfinan, T., Mammano, F., Haseltine, W. A. & Gottlinger, H. G. Role of the matrix protein in the virion association of the human immunodeficiency virus type 1 envelope glycoprotein. J Virol 68, 1689 (1994).
17. Freed, E. O. & Martin, M. A. Virion incorporation of envelope glycoproteins with long but not short cytoplasmic tails is blocked by specific, single amino acid substitutions in the human immunodeficiency virus type 1 matrix. J Virol 69, 1984 (1995).
18. Mammano, F. et al. Rescue of human immunodeficiency virus type 1 matrix protein mutants by envelope glycoproteins with short cytoplasmic domains. J Virol 69, 3824 (1995).
19. Freed, E. O. & Martin, M. A. Domains of the human immunodeficiency virus type 1 matrix and gp41 cytoplasmic tail required for envelope incorporation into virions. J Virol 70, 341 (1996).
20. Cosson, P. Direct interaction between the envelope and matrix proteins of HIV-1. Embo J15, 5783 (1996).
21. Murakami, T. & Freed, E. O. Genetic evidence for an interaction between human immunodeficiency virus type 1 matrix and alpha-helix 2 of the gp41 cytoplasmic tail. J Virol 74, 3548 (2000).
22. Reil, H., Bukovsky, A. A., Gelderblom, H. R. & Gottlinger, H. G. Efficient HIV-1 replication can occur in the absence of the viral matrix protein. Embo J17, 2699 (1998).
23. Franke, E. K., Yuan, H. E. & Luban, J. Specific incorporation of cyclophilin A into HIV-1 virions. Nature 372, 359 (1994).
24. Thali, M. et al. Functional association of cyclophilin A with HIV-1 virions. Nature 372, 363 (1994).
25. Gamble, T.R. et al. Crystal structure of human cyclophilin A bound to the amino-terminal domain of HIV-1 capsid. Cell 87, 1285 (1996).
26. Accola, M. A., Strack, B. & Gottlinger, H. G. Efficient particle production by minimal Gag constructs which retain the carboxy-terminal domain of human immunodeficiency virus type 1 capsid-p2 and a late assembly domain. J Virol 74, 5395 (2000).
27. von Schwedler, U., Stray, K Y & Sundquist, W. Functional surfaces of the HIV-1 CA protein., in preparation (2002).
28. Gamble, T. R. et al. Structure of the carboxyl-terminal dimerization domain of the HIV-1 capsid protein. Science 278, 849 (1997).
29. Worthylake, D. K. et al. Structures of the HIV-1 capsid protein dimerization domain at 2.6 A resolution. Acta Ciystallogr D Biol Crystallogr 55, 85 (1999).
30. Dorfinan, T. et al. Functional domains of the capsid protein of human immunodeficiency virus type 1. J Virol 68, 8180 (1994).
31. Reicin, A. S. et al. Linker insertion mutations in the human immunodeficiency virus type 1 gag gene: effects on virion particle assembly, release, and infectivity. J Virol 69, 642 (1995).
32. Reicin, A. S. et al. The role of Gag in human immunodeficiency virus type 1 virion morphogenesis and early steps of the viral life cycle. J Virol 70, 8645 (1996).
33. Ehrlich, L. S., Agresta, B. E. & Carter, C. A. Assembly of recombinant human immunodeficiency virus type 1 capsid protein in vitro. J Virol 66,4874 (1992).
34. Campbell, S. & Vogt, V. M. Self-assembly in vitro of purified CA-NC proteins from Rous sarcoma virus and human immunodeficiency virus type 1. J Virol 69, 6487 (1995).
35. Groβ, I., Hohenberg, H. & Krausslich, H. G. In vitro assembly properties of purified bacterially expressed capsid proteins of human immunodeficiency virus. Eur J Biochem 249, 592 (1997).
36. Groβ, I., Hohenberg, H., Huckhagel, C. & Krausslich, H. G. N-Terminal extension of human immunodeficiency virus capsid protein converts the in vitro assembly phenotype from tubular to spherical particles. J Virol 72, 4798 (1998).
37. Groβ, I. et al. A conformational switch controlling HIV-1 morphogenesis. Embo J 19, 103 (2000).
38. von Schwedler, U. K. et al. Proteolytic refolding of the HIV-1 capsid protein amino-terminus facilitates viral core assembly. Embo J17, 1555 (1998).
39. Ganser, B. K. et al. Assembly and analysis of conical models for the HIV-1 core. Science 283, 80 (1999).
40. Li, S., Hill, C. P., Sundquist, W. I. & Finch, J. T. Image reconstructions of helical assemblies of the HIV-1 CA protein. Nature 407, 409 (2000).
41. Johnson, J. E. & Speir, J. A. Quasi-equivalent viruses: aparadigm for protein assemblies. J Mol Biol 269, 665 (1997).
42. Fuller, S. D. et al. Cryo-electron microscopy reveals ordered domains in the immature HIV-1 particle. Curr Biol 7, 729 (1997).
43. Yeager, M. et al. Supramolecular organization of immature and mature murine leukemia virus revealed by electron cryo-microscopy: implications for retroviral assembly mechanisms. Proc Natl Acad Sci USA 95, 7299 (1998).
44. Wilk, T. et al. Organization of immature human immunodeficiency virus type 1. J Virol 75, 759 (2001).
45. Wiegers, K. et al. Sequential steps in human immunodeficiency virus particle maturation revealed by alterations of individual Gag polyprotein cleavage sites. J Virol 72, 2846 (1998).
46. Campbell, S. et al. Modulation of HIV-like particle assembly in vitro by inositol phosphates. Proc Natl Acad Sci USA 28, 28 (2001).
47. Gitti, R. K. et al. Structure of the amino-terminal core domain of the HIV-1 capsid protein. Science 273, 231 (1996).
48. Momany, C. et al. Crystal 'structure of dimeric HIV-1 capsid protein. Nat Struct Biol 3, 763 (1996).
49. Berthet-Colominas, C. et al. Head-to-tail dimers and interdomain flexibility revealed by the crystal structure of HIV-1 capsid protein (p24) complexed with a monoclonal antibody Fab. Embo J 18, 1124 (1999).
50. Cornilescu, C. C. et al. Structural analysis of the N-terminal domain of the human T-cell leukemia virus capsid protein. J Mol Biol 306, 783 (2001).
51. Conway, J. F. et al. Virus maturation involving large subunit rotations and local refolding. Science 292, 744 (2001).
52. Wikoff, W. R. et al. Topologically linked protein rings in the bacteriophage HK97 capsid. Science 289, 2129 (2000).
53. Campos-Olivas, R., Newman, J. L. & Summers, M. F. Solution structure and dynamics of the Rous sarcoma virus capsid protein and comparison with capsid proteins of other retrovimses. J Mol Biol 296, 633 (2000).
54. Kingston, R. L. et al. Structure and self-association of the rous sarcoma virus capsid protein. Structure Fold Des 8, 617 (2000).
55. Ehrlich, L. S. et al. HIV-1 capsid protein forms spherical (immature-like) and tubular (mature-like) particles in vitro: structure switching by pH-induced conformational changes. Biophys J 81, 586 (2001).
56. Caspar, D. L. Movement and self-control in protein assemblies. Quasi-equivalence revisited. Biophys J32, 103 (1980).
57. Rossmann, M. G. Constraints on the assembly of spherical virus particles. Virology 134, 1 (1984).
58. Berger, B., Shor, P. W., Tucker-Kellogg, L. & King, J. Local rule-based theory of virus shell assembly. Proc Natl Acad Sci USA 91, 7732 (1994).
59. Zlotnick, A. To build a virus capsid. An equilibrium model of the self assembly of polyhedral protein complexes. J Mol Biol 241, 59 (1994).
60. Prevelige, P. E., Jr. Inhibiting virus-capsid assembly by altering the polymerisation pathway. Trends Biotechnol 16, 61 (1998).
61. Zlotnick, A. et al. A theoretical model successfully identifies features of hepatitis B virus capsid assembly. Biochemistry 38, 14644 (1999).
62. Berthoux, L. et al. Mutations in the N-terminal domain of human immunodeficiency virus type 1 nucleocapsid protein affect virion core structure and proviral DNA synthesis. J Virol 71, 6973 (1997).
63. Tang, S. et al. Human immunodeficiency virus type 1 N-terminal capsid mutants that exhibit aberrant core morphology and are blocked in initiation of reverse transcription in infected cells. J Virol 75, 9351 (2001).
64. Forshey, B., Zhou, J., von Schwedler, U., Sundquist, W. I., Aiken, C. HIV-1 replication requires formation of a viral core of optimal stability., submitted for publication (2002).
65. Grzesiek, S. & Bax, A. J: Am. Chem. Soc. 115, 12593 (1-993).
66. Piotto, M., Saudek, V. & Sklenar, V. Gradient-tailored excitation for single-quantum NMR spectroscopy of aqueous solutions. J Bio mol NMR 2, 661 (1992).
67. Mori, S., Abeygunawardana, C., Johnson, M. O. & van Zijl, P. C. Improved sensitivity of HSQC spectra of exchanging protons at short interscan delays using a new fast HSQC (FHSQC) detection scheme that avoids water saturation. J Magn Reson B 108, 94 (1995).
68. Wittekind, M. HNCACB, A HIGH-sensitivity 3D NMR experiment to correlate amide-proton and nitrogen resonances with the alpha and beta carbon resonances in proteins. J Magn Reson B 101, 201 (1993).
69. Grzesiek, S. & Bax, A. Correlating backbone amide and side chain resonances in larger proteins by multiple relayed triple resonance NMR. J. Am. Chem. Soc. 114, 6291 (1992).
70. Kay, L. E., Xu, G. Y. & Yamazaki, T. Enhanced-sensitivity triple-resonance spectroscopy with minimal H20 saturation. J Mag. Res. 109, 129 (1994).
71. Vuister, G. W. Resolution enhancement and spectral editing of uniformly 13C-enriched proteins by homonuclear broadband 13C decoupling. J Mag. Res. 98,428 (1992).
72. Santoro, J. & King, G.C. A constant-time 2D overbodenhausen experiment for inverse correlation of isotopically enriched species. J. Mag. Res. 97, 202 (1992).
73. Zhang, O., Kay, L. E., Olivier, J. P. & Forman-Kay, J. D. Backbone 1H and 15N resonance assignments of the N-terminal SH3 domain of drk in folded and unfolded states using enhanced-sensitivity pulsed field gradient NMR techniques. J Bio mol NMR 4, 845 (1994).
74. Jeener, J., Meier, B. H., Bachmann, P. & Ernst, R. R. J Chem. Phys. 71, 4546 (1979).
75. Muhandiram, D. R., Xy, G. Y. & Kay, L. E. An enhanced-sensitivity pure absorption gradient 4D 15N, 13C-edited NOESY experimetn. J Biomol. NMR 3, 463 (1993).
76. Pascal, S. M. M., Yamazaki, D. R., Forman-Kay, J. D. & Kay, L. E. Simultaneousl acquisition of 15N- and 13C edited NOE spectr of proteins dissolved in H2O. J Mag. Res. 191, 197 (1994).
77. Vuister, G. W. C. et al. Increased resolution and improved spectral quality of 4D 13C/13C-separated HMQC-NOESY-HMQC spectra using pulsed field gradients. J Mag. Res. B101, 210 (1993).
78. Cornilescu, G., Delaglio, F. & Bax, A. Protein backbone angle restraints from searching a database for chemical shift and sequence homology. J Bio mol NMR 13, 289 (1999).
79. Felix-97. (Biosym Technologies, Molecular Simulations Inc.: San Diego, 1997).
80. Laskowski, R. A. et al. AQUA and PROCHECK-NMR: programs for checking the quality of protein structures solved by NMR. J Bio mol NMR 8, 477 (1996).
81. Wishart, D. S., Sykes, B. D. & Richards, F. M. Relationship between nuclear magnetic resonance chemical shift and protein secondary structure. J Mol Biol 222, 311 (1991).
82. Guntert, P., Mumenthaler, C. & Wuthrich, K. Torsion angle dynamics for NMR structure calculation with the new program DYANA. J Mol Biol 273, 283 (1997).
83. Kraulis, P. Molscript.,(1997).
84. Joshi, S. M. & Vogt, V. M. Role of the rous sarcoma virus p10 domain in shape determination of gag virus-like particles assembled In vitro and within escherichia coli. J Virol 74, 10260 (2000).
85. Massiah, M. A. et al. Three-dimensional structure of the human immunodeficiency virus type 1 matrix protein. J Mol Biol 244, 198 (1994).
86. Gottlinger, H. G., Sodroski, J. G. & Haseltine, W. A. Role of capsid precursor processing and myristoylation in morphogenesis and infectivity of human immunodeficiency virus type 1. Proc Natl Acad Sci USA 86, 5781(1989).
87. Pettit, S.C . et al. The regulation of sequential processing of HIV-1Gag by the viral protease. Adv Exp Med Biol 436, 15 (1998).
88. Pettit, S. C. et al. The p2 domain of human immunodeficiency virus type 1 Gag regulates sequential proteolytic processing and is required to produce fully infectious virions. J Virol 68, 8017 (1994).
89. Matthews, S. et al. Structural similarity between the p17 matrix protein of HIV-1 and interferon-gamma. Nature 370, 666 (1994).
90. Hill, C. P. et al. Crystal structures of the trimeric human immunodeficiency virus type 1 matrix protein: implications for membrane association and assembly. Proc Natl Acad Sci USA 93, 3099 (1996).
91. Wlodawer, A. & Erickson, J. W. Structure-based inhibitors of HIV-1 protease. Annu Rev. Biochem 62, 543 (1993).

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Mature and Immature CA Structures

a) Results

(1) Characterization of MA-CA Fusion Proteins

¹H, ¹⁵N HSQC NMR spectroscopy was used to survey a series of proteins in which MA extensions were fused to the N-terminus of the CA NTD. The intact MA domain appears to inhibit native particle formation in vitro and in vivo (22, 37, 46), but spherical immature particles form efficiently when MA extensions lacking the globular domain (denoted AMA, see FIG. 1B) are fused to either CA or CA-SP1-NC (22). NMR spectra revealed that the ΔMA extension also caused significant chemical shifts in a number of backbone amide protons throughout the N-terminal domain of CA, as compared to the fully processed protein (FIG. 1C).

A series of shorter proteins were then tested to determine the minimal MA extension required to produce this conformational change. MA-CA fusion protein constructs containing only the final 28, 6, and 4 MA residues caused the same set of diagnostic chemical shift changes within CA and therefore presumably effected the same conformational changes (FIG. 1C). In all cases, addition of recombinant viral protease (which cleaves at the MA-CA junction) reverted the spectrum to that of the mature CA NTD, confirming that covalent attachment of the MA extensions was responsible for the conformational change. 3D ¹⁵N-NOESY-HSQC and ¹⁵N-TOCSY-HSQC spectra, together with chemical shift analyses, provided no evidence for order within the MA residues of the longer proteins, and the shortest construct (denoted ₁₂₉M-CA₂₇₈) was therefore selected for full structure determination.

(2) Structure of ₁₂₉MA-CA₂₇₈

The ₁₂₉MA-CA₂₇₈ structure was calculated using 1531 nuclear Overhauser effect (NOE) distance restraints and 161 dihedral angle restraints. The 20 lowest energy structures superimpose on the mean structure with a backbone heavy atom rmsd of 0.55 Å in regions of regular secondary structure (Table 1).

### Table 1 Structural Statistics for ₁₂₉MA-CA₂₇₈

NMR-derived Restraints used in the Structure Calculation

Interproton restraints (total, ave/residue, intraresidue, sequential, medium¹, long-range²) 1531, 10.2, 184, 342, 707, 298
Backbone torsion angle restraints (phi, psi) 161
Hydrogen bond restraints 119
   Statistics for 20 Lowest Penalty Structures³
Average DYANA target function (Å²) 2.0±0.2
Maximum distance violations (Å)(upper limits, lower limits, van der Waals) 0.17, 0.14, 0.32
Maximum torsion angle violation (deg) 4.8
   Coordinate Precision for 20 Lowest Penalty Structures (Å)³
Rmsd of heavy backbone atoms (Å)(secondary structures only, all residues) 0.55±0.08, 2.3±0.5
Rmsd of all heavy atoms (Å)(secondary structures only, all residues) 0.98±0.10, 2.9±0.4
   Procheck Analysis of 20 Lowest Penalty Structures³
Most favored region 73.3%
Additional allowed region 19.4%
Generously allowed region 5.8%
Disallowed region 1.5%⁴
   Table Notes
(1) 2-4 residues.
(2) >4 residues.
(3) 20 lowest penalty structures selected from 220 structures annealed through 16,000 cooling steps. Superpositions are vs. the mean structure.
(4) Residues with dihedral angles in the disallowed region were all in disordered. loops, with the exception of Arg 232 (helix 5) in one of the 20 structures.

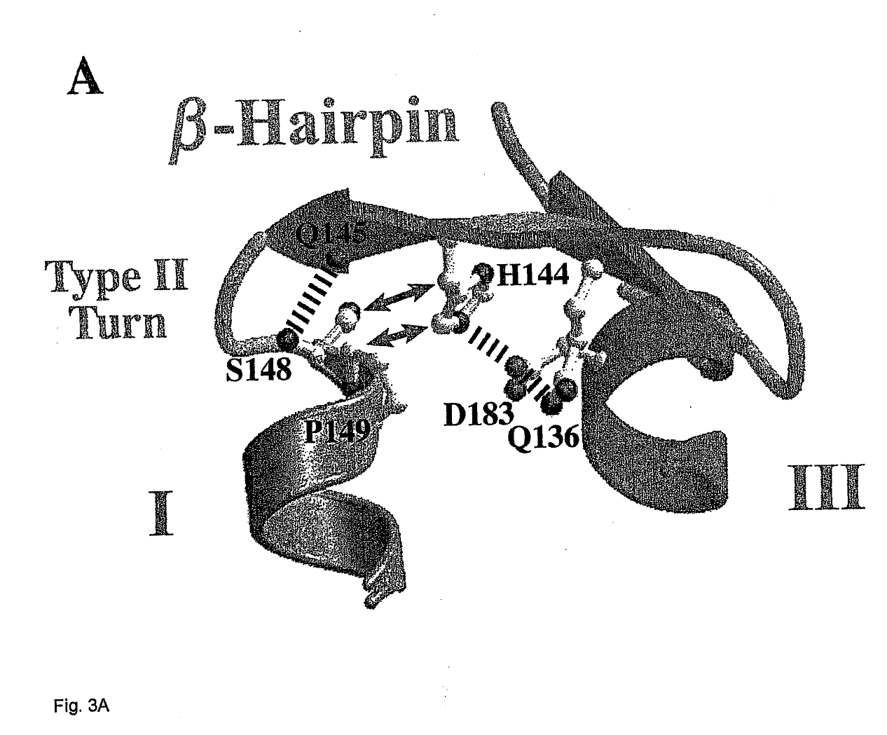

The ₁₂₉MA-CA₂₇₈ structure consists of an N-terminal, two-stranded antiparallel β-sheet (the “β-hairpin”) followed by seven α-helices (FIG. 2). Helices 1-3, 4, and 7 pack in roughly parallel orientations along the long dimension of the domain, spaying apart at the top of the structure to incorporate the perpendicular helices 5 and 6. Seven of the eight loops in the structure are small and well ordered, the exception being an extended loop that connects helices 4 and 5 and contains the cyclophilin A binding site. The protein's N-terminus projects into solution, and the final four MA residues contact helix 6, although these interactions are not extensive. The β-hairpin is oriented down against the globular domain by a type II (glycine) turn located between the hairpin and helix 1, and there are significant packing interactions between β-strand 2 and helices 1 and 3 (shown in FIG. 3A).

(3) Comparison with the Fully Processed CA Structure

Structures of the fully processed CA NTD are in good agreement (25, 47-49), and presumably represent a “mature” CA conformation. Comparison of the ₁₂₉MA-CA₂₇₈ and CA₂₇₈ structures reveals that all of the secondary structural elements are retained (FIG. 2A), but rearrange significantly in the absence of the MA extension. The biggest difference between the ₁₂₉MA-CA₂₇₈ and CA₂₇₈ structures is in the orientation of the N-terminal CA β-hairpin. Upon removal of the MA extension, the hairpin rotates up through an arc of ˜140° and twists about its long axis by ˜90°, displacing loop residues by up to 30Å. This is referred to as the “hairpin up” conformation, because the β-hairpin loop projects up and away from the domain. Residues Ile 147 and Ser 148 form the pivot points for rotation of the hairpin via changes in their backbone torsion angles. These two residues initially form the C-terminal half of the type II turn between the hairpin and helix 1 in the ₁₂₉MA-CA₂₇₈ structure, and then rotate to extend strand 2 (Ile 147) and cap helix 1 (Ser 148) in the mature protein.

Helices 1, 3, and 6, which surround the β-hairpin, also reorient significantly upon processing at the MA-CA junction (FIG. 3B, C). Remarkably, the packing register between helices 1 and 2 changes by one full helical repeat, with helix 1 displaced toward its N-terminus in the mature structure. Helix 1 also shifts toward the N-terminal end of the parallel helix 3. In spite of these movements, residues in helix 1 generally maintain analogous pairwise interactions with helix 2 and 3 residues, although their side chain packing interactions change significantly. Smaller adjustments in helices 3 and 6 are also observed, and appear coupled to the larger movements of the β-hairpin and helix 1. In particular, the axis of helix 6 tilts, allowing its N-terminus to buttress the base of the β-hairpin in both structures, while maintaining C-terminal packing interactions with helix 7 (FIG. 3B).

(4) A Salt Bridge Between Asp183 and His144

Analysis of the ₁₂₉M-CA₂₇₈ structure suggested that Asp 183, which forms a salt bridge with the N-terminus of the processed CA protein in the hairpin up conformation, might also form a salt bridge in the hairpin down conformation, in this case with the protonated side chain of His 144. At pH values less than 6.3 (structure determined at pH 5.5), all five CA histidines are protonated. This determination was based upon nitrogen and proton chemical shifts, as well as coupling patterns in HNBC spectra (Pelton et al 1993 and Blomberg et al. 1997). Since the histidine ring nitrogen shifts (Nδ1 and Nε2) are nearly the same at low pH (charged state), this form is easily distinguished from either of the two neutral tautomers. A pH titration was performed and revealed that the pKa of His 144 is elevated to at least pH 8.0 by the local protein environment, consistent with the postulated His 144 . . . Asp 183 salt bridge (FIG. 4A). Three of the remaining five histidines (195, 217 and 252) exhibited normal pKa values (˜6.7), and the pKa of His 219 was elevated slightly. Chemical shift changes indicated that His 144 does begin to deprotonate near pH 8.0, but it was not possible to complete the titration because the protein simultaneously aggregated and precipitated from solution, perhaps because the P-hairpin unfolds upon deprotonation of His 144.

As the ₁₂₉MA-CA₂₇₃ structure suggested the possibility that protonation of His 144 might serve as a conformational switch, the pH dependence of CA assembly was tested. CA spontaneously forms long helical tubes when incubated at pH 8 under high protein and ionic strength conditions (FIG. 4B). Conical structures are also occasionally observed under these conditions, but are rare. At pH 6.0, however, the CA tubes are significantly shorter, and cones are much more prevalent. Thus, it was concluded that pH does alter CA assembly in vitro, with more acidic conditions favoring structures with greater curvature. Our attempts to test whether His 144 was directly involved in this process were inconclusive because the CA H144A mutant protein did not assemble well under any conditions tested.

The closed, asymmetric shapes and structural polymorphism of the HIV-1 Gag and capsid shells imply that these proteins must adopt multiple conformations as the virus proceeds through its replication cycle. The ₁₂₉MA-CA₂₇₃ structure has revealed a second stable conformation for the N-terminal domain of CA, which is favored by even short MA extensions and by acidic conditions. Both of these conditions can also alter the morphology of CA assemblies formed in vitro, indicating that local conformational changes at the N-terminal end of CA can be propagated to change protein's higher order interactions. Hence, the CA hairpin down conformation reported here is consistent with an important role in viral capsid assembly in vivo.

The β-hairpin switch alters the packing register between helices 1 and 2. A pseudomolecular model for the structure of the mature HIV-1 capsid based upon cryo-EM reconstructions and modeling studies of the helical tubes formed by the CA protein has previously been proposed. In docked models, the N-terminal domain of CA forms a hexamer that is stabilized by intermolecular packing of helices 1 and 2 to form a hexameric ring (40). This is consistent with CA hexamerization being sensitive to the disposition of these helices and that the hairpin down configuration will disfavor hexamerization.

(5) The β-hairpin Switch

Comparison of the two CA conformations reported to date demonstrates the striking conformational flexibility of the N-terminal CA β-hairpin, since this element swings up through an arc of ˜140° upon proteolysis at the MA-CA junction. Helices 1 and 2 also shift register, and these two structural changes appear to be coupled because the upward rotation of the hairpin would “pull” up on helix 1, while simultaneously blocling the top of helix 2 and thereby preventing it from tracking along with helix 1. The analogous 1-hairpin at the N-terminus of the CA protein of HTLV-I adopts an orientation that is roughly half way between the two extremes observed so far for HIV-1 CA (50). The reorientation of an extended β-hairpin is also an important element in the conformational polymorphism exhibited by the gp5 coat protein of HK97 phage (51, 52). In that case, the orientation of the extended “E-loop” hairpin differs between the hexamer and pentamer conformations in the Head II structure, and also changes as individual gp5 subunits move during viral maturation. Thus, use of rotating β-hairpin “lever arms” may be used to accommodate conformational changes in many viral coat proteins.

(6) Interactions that Stabilize the Different CA Conformations

Although both hairpin conformations seem accessible to the fully processed CA protein, the hairpin up predominates in solution (47). This is presumably because N terminus makes a series of important contacts that stabilize the conformation. Specifically, the protonated Pro 133 amine forms a partially buried salt bridge with the side chain carboxylate of Asp183 and the proline ring binds in pocket defined by Ile 147, Gly178, and Gln145 (FIG. 3A). These interactions appear functionally important because mutation of Asp183 (to Ala) blocks viral-capsid assembly and replication. Moreover, the Pro133 and Asp183 pair is nearly invariant across retroviruses (38), and analogous salt bridges are observed in CA NTD of Rous sarcoma virus (53, 54).

MA extensions disfavor the hairpin up conformation because they remove the positive charge on the Pro 133 amine (now an amide) and create steric hindrance, which is removed by rotation of the β-hairpin. The hairpin down conformation appears to be stabilized by favorable packing interactions between hairpin strand 2 and helices 1 and 3, including the His 144 . . . Asp183 salt bridge. However, the type II turn that precedes the hairpin is likely to be energetically unfavorable because it has an Ile (rather than Gly) residue in the third position.

(7) pH Dependent Structural Changes

NMR is ideally suited for the study of protonation states in proteins, and can provide information on the tautomeric state of each observable histidine residue. Disclosed herein, His 144 is protonated at neutral pH in the hairpin down conformation, allowing the Asp 183 salt bridge. His 144 begins to deprotonate appreciably above pH 8, however, and this likely causes the hairpin structure to unfold. This agrees well with biochemical analyses of Groβ et al, who showed that the ΔMA-CA-NC-SP2 protein assembles into “mature” cones and tubes at pH 6, but forms “immature” spheres at pH 8 (37). This morphological switch correlates with pH dependent conformational changes within the N-terminal domain of CA as detected by monoclonal antibodies against defined linear epitopes. Two different antibodies that bind to determinants in helices 3 and 6 failed to recognize the protein at acidic pH, but bound well at alkaline pH. Our studies indicate that this is because antibody binding is sterically occluded at low pH when the MA extension packs down against helices 3 and 6, but allowed when the hairpin unfolds at high pH. In summary, it appears that residues in the CA hairpin region can adopt at least three energetically accessible conformational states: i.e., hairpin up, hairpin down, and hairpin unfolded (FIG. 5A).

Two other groups have tested the pH dependence of CA assembly in vitro, albeit under slightly different assembly conditions than those reported here. GroB et al reported that CA tube assembly was more efficient at alkaline pH, but noted no morphological changes as a function of pH (35). In contrast, Ehrlich et al used light scattering experiments to measure the ratio of particle size:hydrodynamic radii, and concluded that CA assembles into spherical particles at acidic pH (low ratio) and tubes at basic pH (high ratio)(55). The results disclosed herein can provide an alternative explanation for the increased particle size: hydrodynamic ratio at lower pH because disclosed herein long helical tubes predominated at pH 8.0 whereas shorter tubes and cones predominated at pH 6. This altered distribution of structures further suggests that the ratio of CA pentamers (which promote curvature) to CA hexamers (the building block of the long helical tubes) can increase at lower pH. As noted above, the hairpin down conformation is also favored by acidic conditions and therefore the hairpin down conformation is an attractive candidate for the conformation of CA in its pentameric state.

b) Methods

(1) Structural Studies

ΔMA-CA_{278, 105}MA-CA₂₇₈ and ₁₂₇MA-CA₂₇₈, and ₁₂₉MA-CA₂₇₈ were expressed and purified (38), and their purity and composition was analyzed by SDS-PAGE and electrospray mass spectrometry. NMR experiments were performed (26°, 1.0-1.5 mM protein, 25 mM sodium phosphate buffer (pH 5.5), 2 mM DTT and 10% D₂O) on unlabeled protein (100% D₂O), ¹⁵N-labeled protein (10% D₂O) and ¹³C/¹⁵N labeled protein (10% D₂O). Spectra were collected on Varian Unity 500 and Inova 600 MHz spectrometers equipped with Nalorac IDTG (¹H, ¹³C, ¹⁵N) triple resonance probes with z-axis pulsed-field gradients.

Solvent suppression was accomplished in all experiments using a water-flip-back pulse (65) and field gradient pulses (66). Sequential backbone assignments were made using the following NMR experiments: ¹⁵N HSQC (67), HNCACB (68), CBCACONH (69) and HNCO (70). Sidechain assignments were made using the following experiments: homonuclear 2D TOCSY (in D₂O), ¹³C CT-HSQC (71, 72), 3D ¹⁵N-TOCSY-HSQC (73), ¹³C-HCCH-TOCSY, ¹³C/¹⁵N H(CCO)NH, ¹³C/¹⁵N-C(CO)NH. The following NOE data were used to generate distance restraints: 2D homonuclear NOESY (74), 3D ¹⁵N-NOESY-HSQC (67,73), 3D ¹⁵N/¹⁵N-HSQC-NOESY-HSQC (75), 3D ¹³C-NOESY-HSQC (75,76), 4D ¹⁵N/¹³C HSQC-NOESY-HMQC (75) and 4D ¹³C/¹³C-HMQC-NOESY-HMQC (77). NOESY mixing times were 100 ms and TOCSY mixing times were 60 ms. Dihedral angle restraints were derived from chemical shift indices using the program TALOS (78). Raw data were processed off line using Felix 97 (79). Secondary structural elements were defined by combining data from PROCHECK-NMR (80), chemical shift indices (81), and hydrogen-bonding patterns. Structures were calculated using DYANA (82), energy minimized using CNS, assessed with PROCHECK-NMR, and displayed using MolScript (83).

(2) pH Titrations

Assignments for the Nδ1, Hδ2, Nε2 and Hε1 resonances of the 5 His residues in ₁₂₉MA-CA₂₇₈ were obtained from a long-range ¹H/¹⁵N HSQC experiment. The delay in which the 1H 15N signals become antiphase was set to 22ms to refocus the magnetization arising from the J_{NH} one-bond amide nitrogen-proton couplings. The nitrogen transmitter was set at 190 ppm while the proton transmitter was placed on water (4.77 ppm). A series of ten long-range HSQC were collected for ₁₂₉MA-CA₂₇₉ (0.5 mM in 20 mM NaPi, 1 mM DTT, 10%OD₂O) at ten different pH values (5.38, 5.61, 5.90, 6.33, 6.50, 6.74, 7.13, 7.37, 7.58, and 7.81). The pH was adjusted with small additions of 10-100 mM HCl or NaOH (10% D₂O) without accounting for deuterium isotope effects. The ₂₉MA-CA₂₇₈ protein aggregates and precipitates at pH values lower than 5.3 and higher than ˜8.0.

(3) Structural Comparisons

Comparisons between the hairpin up and down conformations were made by superimposing all backbone heavy atoms of helices 2 (168-174), 4 (196-213), and 7 (260-277) of the lowest penalty ₁₂₉MA-CA₂₇₈ structure with the same atoms in the 1.9Å crystal structure of ₁₃₃CA₂₇₈ (1.23 Å rmsd) (ref. 28 and F. Vajdos, personal communication). Note that the relative backbone atom position shifts in helix 1 were even larger when the ₁₂₉MA-CA₂₇₈ structure was compared to the solution structure of CA₁₃₃-₂₈₃ (47).

(4) CA Assembly Reactions

Assembly reactions were performed at 37° C. for 1 hour under the following conditions: 400 mM CA, 50 mM MES (pH 6.0), 1 M NaCl or 400 mM CA, 50 mM Tris (pH 8.0) 1 M NaCl. NMR assignments and atomic coordinates for ₁₂₉MA-CA₂₇₈ have been deposited in the Protein Database, and chemical shift data have been deposited to the BRMB and are herein incorporated by reference in their entireties for the material related to the structure of ₁₂₉MA-CA₂₇₈.

### 2. Example 2

### Inhibitor Development

To build a viral capsid structure of the correct morphology, the assembling subunits must have several energetically accessible conformations, and switch correctly and with high fidelity (56-59). Hence, the development of inhibitors that alter capsid assembly pathways can represent an attractive strategy for the development of new antivirals (60, 61). Even in wild type HIV-1, a large percentage of viral capsids exhibit aberrant morphologies, suggesting that accurate capsid assembly may represent a challenge for the virus. Moreover, all CA and NC mutations identified to date that inhibit capsid assembly or alter capsid stability also block viral replication (27, 30, 32, 62-64), indicating that small molecules that altered HIV-1 capsid assembly would likely inhibit viral replication.

Capsid assembly could be altered by small molecules that bound specifically and stabilized the hairpin down conformation. The surface topology of the protein exhibits two unique cavities that are possible binding sites for such small molecule inhibitors. The larger (˜600 Å³), corresponds to the approximate binding site for Pro 133 in the hairpin up conformation (FIG. 5B). The His144 . . . Asp183 salt bridge forms the base of this cavity, and the other residues that define the walls are generally well conserved in different HV isolates. Thus the size, conservation, and apparent functional importance of the cavity make it a target for inhibitor design.

a) Materials and Methods

(1) DNA Constructs

The gene encoding HIV-1_{NL4-3} Gag was mutated at codon 247 (Ile to Cys with primer 5′TGTCATCCATCCGCATTGTTCCTGAAG 3′; SEQ ID NO:29) using single-stranded mutagenesis by the Kunkel method (Kunkel et al., 1987). DNA encoding ₁₀₅MA-CA₂₇₈ (Gag residues 105 to 278) and CA₁₃₃₋₂₇₈ (133 to 278) with I247C mutation were amplified by PCR and subcloned into the NdeI/XhoI site of pET32a (Novagen) vector, which encodes an C-terminal (His)₆ sequence and was modified to contain an in-frame NdeI restriction site (forward primers 5′ GGATCGGATATACATATGGAAGAAGAACAAA ACAAAAGTAAG 3 ′ (SEQ ID NO:30) and 5′ GGATCGCCGCACCATATGCCGATCGTGCAGAACCT CCAGGGG 3∝ (SEQ ID NO:31), reverse primer 5′ GAATGCTCTCGAGGCTATACATTCTTACTATTTT 3′ (SEQ ID NO:32)). The resulting plasmids WISP0093 (encoding ₁₀₅MA-CA₂₇₈(His)₆) and WISP0099 (encoding CA₃₃₂₋₂₇₈(His)₆) were confirmed by dideoxy sequencing.

(2) Protein Expression and Purification

The ₁₀₅MA-CA₂₇₈(His)₆ and CA₁₃₃₋₂₇₈(His)₆proteins were expressed in freshly transformed BL21(DE3) cells (2 liters of culture, 4-hour induction with 0.4 mM isopropyl-β-D-thiogalacopyranoside, A₆₀₀˜0.4). Cells were harvested by centrifugation, resuspended in 50 ml of buffer A (25 mM Tris-HCl (pH 7.5), 300 mM NaCl, 10 mM Imidazole, 2 mM 2-mercaptoethanol), and lysed in a French press (this and all subsequent steps were performed at 4° C.). The cell lysate was sonicated to reduce viscosity, and centrifuged for 50 min at 39, 200g to remove insoluble cellular debris. The His-tagged protein was affinity purified on a 20 ml TALON™ Metal Affinity Resin column with immobilized Co²⁺ (Clontech). The protein was eluted at ˜150 mM imidazole from a linear gradient of 10 mM to 200 mM imidazole in buffer A. Fractions containing the protein were pooled, dialyzed overnight in 2 liters of buffer B (25 mM Tris-HCl (pH 8.0), 10 mM 2-mercaptoethanol), and chromatographed on a Q-Sepharose column (Pharmacia). The protein was eluted at ˜100 mM NaCl from a linear gradient of 0 to 1 M of NaCl in buffer B. Eluted protein was pooled, dialyzed overnight against 2 liters of buffer B, and concentrated in an Amicon centriprep.

The purified ₁₀₅MA-CA₂₇₈(His)₆ and CA₁₃₃₋₂₇₈(His)₆proteins were characterized by electrospray mass spectrometry (MW_{obs}=20,449 g/mol, MW_{calc}=20, 453 g/mol for ₁₀₅MA-CA₂₇₈(His)₆; MW_{obs}=17,240g/mol, MW_{calc}=17,243 g/mol for CA₁₃₃₋₂₇₈(His)₆).

(3) [³H] N-Ethylmaleimide (NEM) Labelinq

The ₁₀₅MA-CA₂₇₈(His)₆ and CA₁₃₃₋₂₇₈(His)₆proteins were reduced with 10 mM DTT for 30 min at 37° C. Excess DTT was removed by dialysis under N₂ against 10 mM phosphate buffer, pH 7.0, containing 50 mM NaCl. After dialysis, the free thiol concentrations were measured by the absorbance at 412 nm in a buffer containing 0.4 mg/ml 5,5′-dithiobis(2-nitrobenzoic acid) (DTNB), 100 mM phosphate (pH 7.2), 150 mM NaCl, and 1 mM EDTA (Ellman method) (Ellman, 1959). An equinolar mixture of ₁₀₅MA-CA₂₇₈(His)₆ and CA₁₃₃₋₂₇₈ (His)₆proteins (20 μM each) were labeled with 1 μM of [³H]NEM (NEN Life Science Products) for 1 hour (h) on ice. The reaction was carried out in 20 mM HEPES buffer, pH 7.0, containing 150 mM NaCl and 2 mM EDTA. The reaction was stopped by the addition of 2-mercaptoethanol to 1 mM. The two proteins were separated by SDS-PAGE, transferred to PVDF membrane (Applied Biosystem), stained with Coomassie blue, and exposed to film (Kodak).

b) Results

NMR structures have revealed that the conformation of the N-terminal domain of CA changes dramatically when four MA residues are added to its N-terminus. These two CA conformations (CA₁₃₃₋₂₇₈ and ₁₂₉MA-CA₂₇₈) differ primarily in the orientations of the N-terminal β-hairpin and the surrounding helices 1, 3, and 6. In addition, a prominent cavity (˜600 Å³) in the structure of ₁₂₉MA-CA₂₇₈ is filled in the structure of CA₁₃₃₋₂₇₈ by the new N-terminus formed upon removal of the MA residues. Disclosed are assays and compositions which determine whether small molecules bind in the cavity and block the conformational change. To screen for small-molecule inhibitors of the structural transition, a chemical probing assay is disclosed that can differentiate between CA in its two conformations.

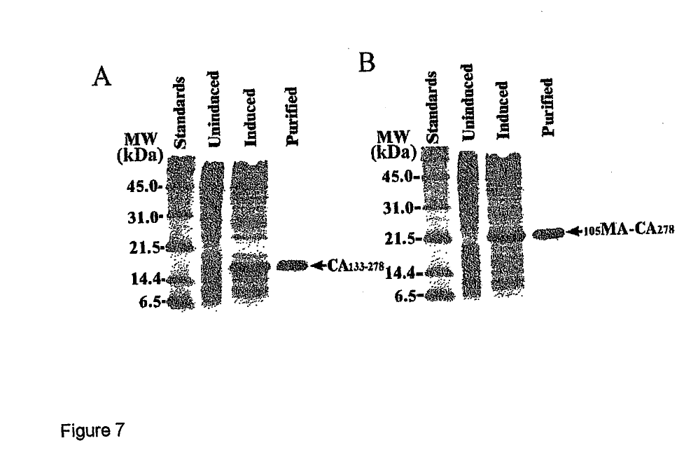

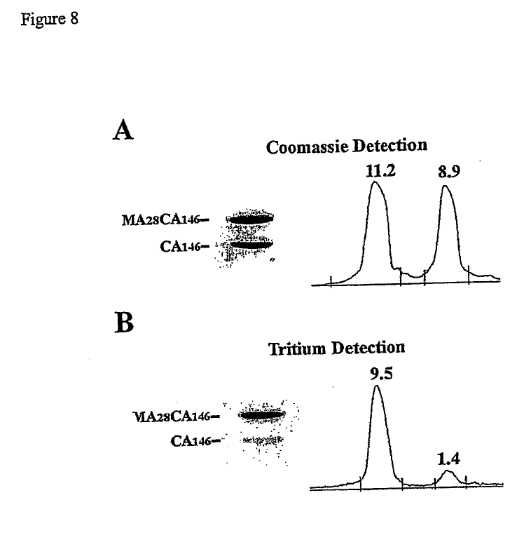

The N-terminal β-hairpin packs down against the globular domain in the ₁₂₉MA-CA₂₇₈ structure, whereas it springs up and packs against helix 6 in the CA₁₃₃₋₂₇₈ structure. As a result, several residues in helix 6 are more exposed in the ₁₂₉MA-CA₂₇₈ structure. Ile247 was mutated to a Cys in CA helix 6 for use in chemical probing analysis. The mutant ₁₀₅MA-CA₂₇₈(His)₆ and CA₁₃₃₋₂₇₈ (His)₆proteins were expressed and purified first (FIG. 7), and then the relative accessibility of Cys247 was tested by chemical probing with ³H-N-ethyl maleimide. The proteins were mixed in equimolar concentrations, reacted with [³]NEM, separated by SDS-PAGE, and detected by Coomassie blue staining (FIG. 8A) and fluorography (FIG. 8B). The Coomassie staining of ₁₀₅MA-CA₂₇₈(His)₆ is 20% darker than that of CA₁₃₃₋₂₇₈(His)₆, probably owning to the ˜20% greater mass of ₁₀₅MA-CA₂₇₈(His)₆. The fluorography analysis shows that the ₁₀₅MA-CA₂₇₈(His)₆ protein incorporates approximately 7-fold more ³H than the CA₁₃₃₋₂₇₈(His)₆protein. Thus, NEM reacts more readily with the “immature” conformation as designed. To rule out the possibility that the differential reactivity may reflect Cys247 oxidation, the two proteins were incubated under reducing conditions and near full Cys247 reduction was confirmed prior to the reaction (free thiol contents were 105±2% for CA₁₃₃₋₂₇₈ (His)₆ and 89±4% for ₁₀₅MA-CA₂₇₈(His)₆). CA₁₃₃₋₂₇₈(His)₆ alone, in the absence of competition from ₁₀₅MA-CA₂₇₈(His)₆, also reacted poorly with [³H]NEM, further indicating the intrinsic lack of reactivity of Cys247 in CA₁₃₃₋₂₇₈(His)₆. These observations, indicate that the differential chemical reactivity of Cys247 in the two CA proteins reflects the different environments in the two CA conformations, with the residue exposed in ₁₀₅MA-CA₂₇₈(His)₆ but less accessible in CA₁₃₃₋₂₇₈(His)₆. Therefore, this chemical probe assay can be used to detect the CA conformational change. Furthermore, the assay can be adapted for high-throughput screening of small molecules that inhibit the structural transition.

### 3. Example 3

### CA-NC Assembly Assay

a) Expression and Purification of Recombinant CA-NC Protein

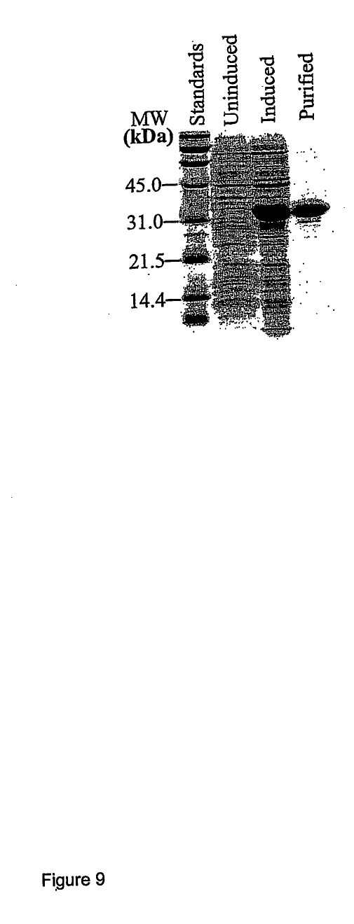

DNA encoding CA-NC protein (HIV-1_{NL4-3} Gag amino acids 133-433) with the point mutation G94D was introduced into the pET11a expression vector (Novagen). The resulting plasmid (WISP9868) was transformed into BL21(DE3) cells. The CA-NC(G94D) protein was expressed by 3-hour induction with 0.5 mM isopropyl-β-D-thiogalacopyranoside at room temperature (optical density at 600 nm (OD₆₀₀)=0.5). Cells (6 liters of culture) were harvested by centrifigation, resuspended in 60 ml of 0.5 M NaCl in buffer A [20 mM Tris-HCl (pH 7.5), 1 μM ZnCl₂, 10 mM 2-mercaptoethanol, 2 tablets of protease inhibitor (Boehringer Mannheim)] (this and all subsequent steps were performed at 4° C.). Cells were lysed by two passes through a French press, and then sonicated to reduce viscosity. Nucleic acids were precipitated from the lysate by the addition of 0.11 equivalents (v/v) of 0.2 M (NH₄)₂SO₄, followed by addition of the same volume of 10% polyethylenimine (pH 8.0). The mixture was stirred on ice for 20 min. Insoluble cellular debris and precipitated nucleic acids were removed by centrifugation at 25, 900 g for 15 min. Crude CA-NC(G94D) protein was precipitated by the addition of 0.35 equivalents saturated (NH₄)₂SO₄ solution, stirred on ice for 15 min, and collected by centrifugation at 9,820 g for 10 min. The pellet was redissolved in 40 ml of 0.1 M NaCl in buffer A, dialyzed twice against 2 liters of 0.05 M NaCl in buffer A, and clarified by centrifugation and filtration through a 0.2-μm filter. The protein was chromatographed on an SP-Sepharose column (Pharmacia) and eluted at ˜400 mM NaCl from a linear gradient of 0.05 to 1 M NaCl in buffer A. Fractions containing the protein were pooled, dialyzed overnight against 2 liters of buffer A, and concentrated in an Amicon centriprep. The expression and purification of CA-NC(G94D) protein were analyzed on a 15% SDS-PAGE, and stained with Coomassie blue (FIG. 9).

b) In vitro Assembly and Electron Microscopy Analysis

Oligonucleotides d(TG)₅₀ (50 repeats of alternating TG sequence) were synthesized at the University of Utah oligonucleotide core facility. The assembly was performed with incubation of the CA-NC(G94D) protein with d(TG)₅₀ for 16 h at 4° C. under the following conditions: 0.3 mg/ml (9 μM) CA-NC(G94D), 0.03 mg/ml (1 μM d(TG)₅₀ (approximately 11 nt/l protein molecule), 500 mM NaCl, 50 mM Tris-HCl.

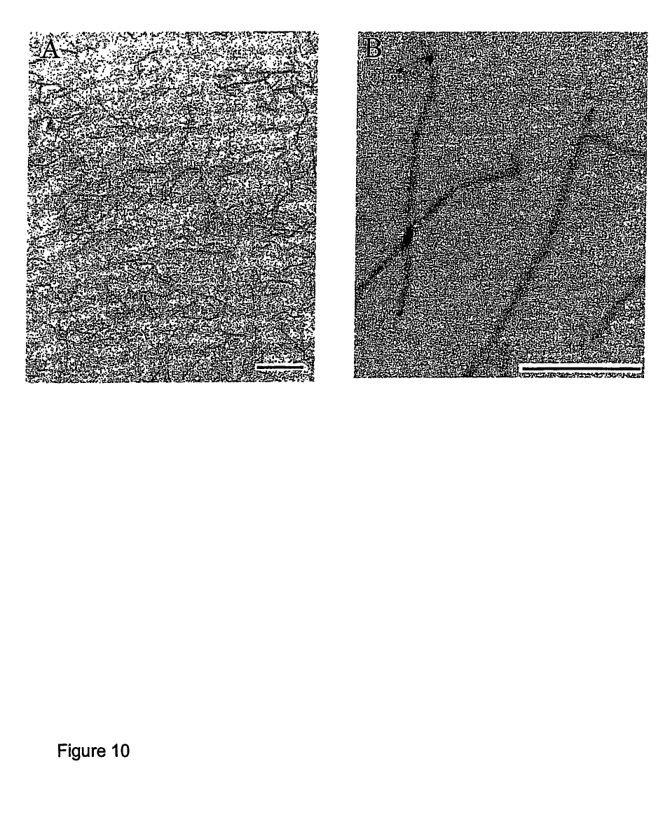

Cylinder formation was monitored by TEM in negatively stained samples (FIG. 10). For staining, 7.5 μl of assembled sample was applied on parafilm and covered with a Formvar/carbon-coated grid (200 μm in mesh size) for 30 sec, washed 3 times with a drop of 0.1 M KCl, and stained 3 times with a drop of 4% uranyl acetate. Cylinder formation was also measured by light scattering at 312 nm (Abs₃₁₂=0.3-0.4 with a pathlength of 1 cm).

### 4. Example 4

### CA Dimerization Assay

a) Expression Plasmids

DNA encoding the C-terminal domain of HIV-1 CA protein (HIV-1_{NL4-3} Gag amino acids 278-354) was amplified by the polymerase chain reaction (PCR) with a forward primer containing two restriction sites (NdeI and NcoI) and a reverse primer containing a BamHI site. The restricted product was ligated and cloned in-frame into the NdeI/BamHI sites of pET11a (Novagen), and the resulting plasmid is WISP0069. The C-terminal CA was amplified again with an NdeI site and an NcoI site introduced to the 5′ and 3′ ends of the gene, respectively. The restricted product was then ligated and cloned in-frame into the NdeI/NcoI sites of WISP0069. The resulting plasmid WISP0070 contains two copies of the CA C-terminal dimerization domain in tandem with an NcoI site in between.

DNA encoding C-terminal CA protein was extended by PCR at the 3′ end with the sequence encoding affinity FLAG epitope tag (DYKDDDK), and with NcoI and BamHI sites introduced to the 5′ and 3′ ends of the gene, respectively. The restricted product was ligated and cloned in-frame into the NcoI/BamHI sites of WISP0070. The resulting plasmid WISP00149 contains two copies of CA C-terminal domain with a C-terminal FLAG tag. The sequences of plasmids were all confirmed by dideoxy sequencing.

b) Expression and Purification of Recombinant Proteins

The expression and purification procedure was the same for both proteins, (CA-CTD)₂ MISP0070) and (CA-CTD)₂-FLAG (WISP00149). Protein was expressed in freshly transformed BL21(DE3) cells [2 liters of culture, 4-hour induction with 1 mM isopropyl-β-D-thiogalacopyranoside, A₆₀₀˜0.4]. Cells were harvested by centrifugation, resuspended in 50 ml of 50 mM NaCl in buffer A [25 mM Tris-HCl (pH 8.0), 5 mM 2-mercaptoethanol], and lysed in a French press (this and all subsequent steps were performed at 4° C.). The cell lysate was sonicated to reduce viscosity, and centrifuged for 50 min at 39,200 g to remove insoluble cellular debris. Protein was precipitated by the addition of saturated (NH₄)₂SO₄ solution to 50%(v/v), stirred on ice for 15 min, and collected by centrifugation at 9,820 g for 10 min. The pellet was redissolved in 40 ml of buffer A, and dialyzed overnight in 2 liters of buffer A. Protein was chromatographed on a Q-Sepharose column (Pharmacia), and eluted at 200 mM NaCl from a linear gradient of 0 to 1 M of NaCl in buffer A. Eluted protein was dialyzed overnight in 2 liters of 1 M (NH₄)₂SO₄ in buffer A, and chromatographed on a Phenyl-Sepharose column (Pharmacia). The protein was eluted at 500 mM (NH4)₂SO₄ from a linear gradient of 1 to 0 M of (NH₄)₂SO₄ in buffer A. Eluted protein was pooled, dialyzed overnight against 2 liters of buffer A, and concentrated by an Amicon centriprep. FIG. 11 shows the expression and purification of (CA-CTD)₂ and (CA-CTD)₂-FLAG.

The purified (CA-CTD)₂ and (CA-CTD)₂-FLAG proteins were characterized by electrospray mass spectrometry (MW_{obs}=17,599 g/mol, MW_{calc}=17,602 g/mol for (CA-CTD)₂; MW_{obs}˜18,709 g/mol; MW_{calc}=18,711 g/mol for (CA-CTD)₂-FLAG).

c) Dimerization Assays by Gel Filtration and Equilibrium Sedimentation

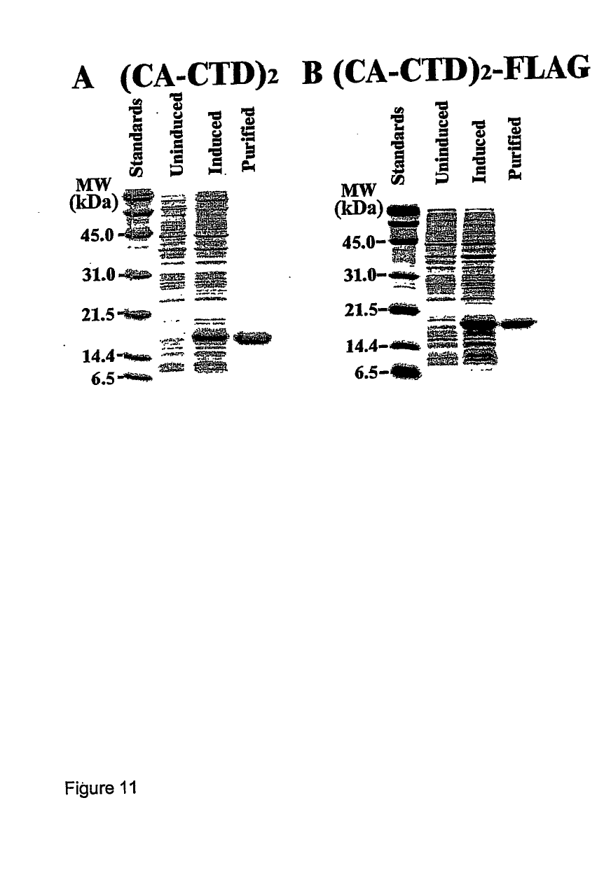

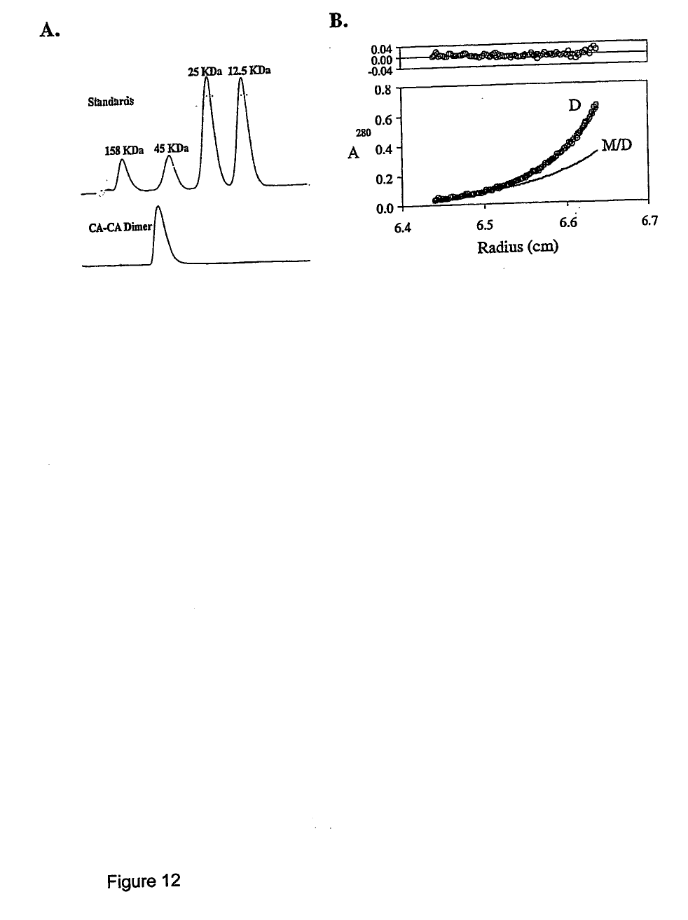

Superdex 75 gel filtration column (Pharmacia) was used to determine the oligomerization state of (CA-CTD)₂ (FIG. 12A). 1 ml of (CA-CTD)₂ protein was loaded at a concentration of 4 mg/ml in 25 mM phosphate (pH 7.2), 150 mM NaCl, 2 mM 2-mercaptoethanol. Protein standards were run under the same conditions. A plot of relative elution volume versus the logarithm of molecular mass was carried out using the protein standards, and an apparent mass of 59 kDa was obtained for (CA-CTD)₂.

Analytic ultracentrifigation was used to quantify the oligomerization state of (CA-CTD)₂. Centrifugation experiment was performed on a Beckman Optima XL-A ultracentrifuge at rotor speed of 22,000 rpm. (CA-CTD)₂ protein was centrifuged at 4° C. in 25 mM phosphate buffer, pH 7.0, containing 100 mM NaCl and 2 mM DTT. Equilibrium distributions were fitted to single homogeneous species assuming a simple dimer model with a fixed protein mass of 35.2 kDa (FIG. 12B). The data demonstrate that the dissociation constant for a monomer-dimer equilibrium must be less than K_{d}=10⁻⁶ M (and probably much less than this). A solvent density of 1.00785 g ml⁻¹ and a partial specific volume of 0.7252 ml g⁻¹were used.

### 5. Example 5

### The CA G94D Mutant CA-NC Protein Assembles into Longer Cylinders Than Wild Type CA-NC

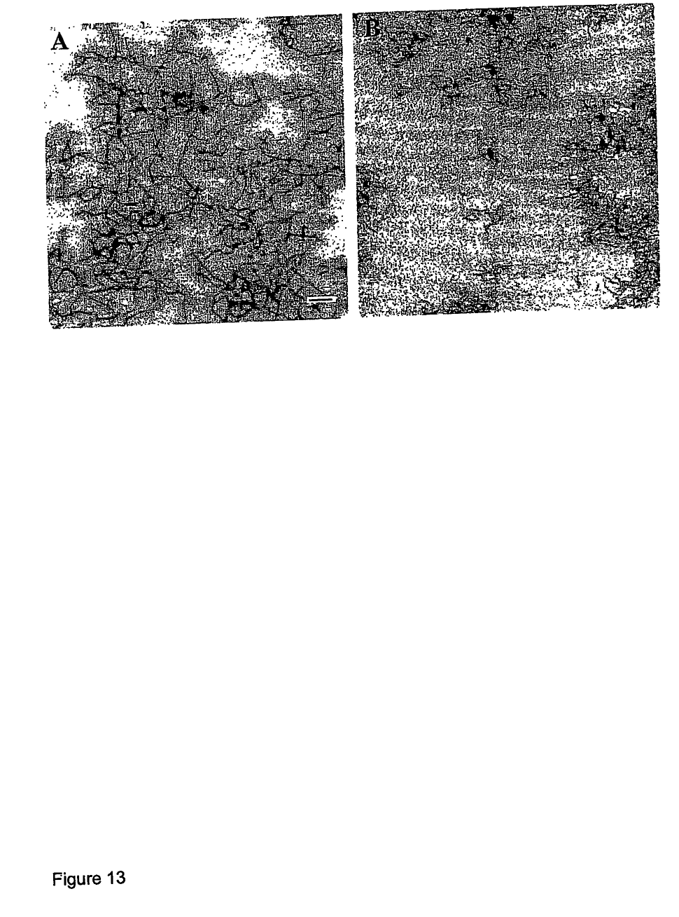

Previous in vitro assembly studies have shown that the CA G94D mutant protein forms longer cylinders than wild-type CA (Li et al., 2000). We therefore tested the assembly of both the CA G94D mutant and wild-type CA-NC on a d(TG)₅₀ template. Reactions were carried overnight at 4° C. under the following conditions: 0.3 mg/ml (9 μM) of protein, 0.03 mg/ml (1 μM of d(TG)₅₀ (approximately 11 nt/l protein molecule), 500 mM NaCl, 50 mM Tris-HCI (pH 8.0). Cylinder formation was measured by light scattering at 312 nm (Abs₃₁₂) (Table 3) and by negatively stained EM (FIG. 13).

TABLE 3
G94D and wild-type CA-NC assembly.

| Protein | Abs₃₁₂ |
|---|---|
| CA-NC (G94D) | 0.292 ± 0.029 |
| CA-NC (wild-type) | 0.735 ± 0.015 |

The light scattering signal of the assembly of wild-type CA-NC is significantly higher than that of the G94D mutant. However, EM images show that G94D mutant protein assembled into long cylinders (FIG. 13A) while wild-type CA-NC formed short cylinders that tended to aggregate (FIG. 13B). This aggregation explains the higher light scattering signal of wild-type CA-NC compared to the G94D mutant. For cylindrical formation the G94D mutant is preferred, and for aggregation formation wild type CA-NC is preferred.

### 6. Example 6

### CA-NC Assembly is Dependent on the Sequence and Length of the Single-Stranded Oligodeoxynucleotides

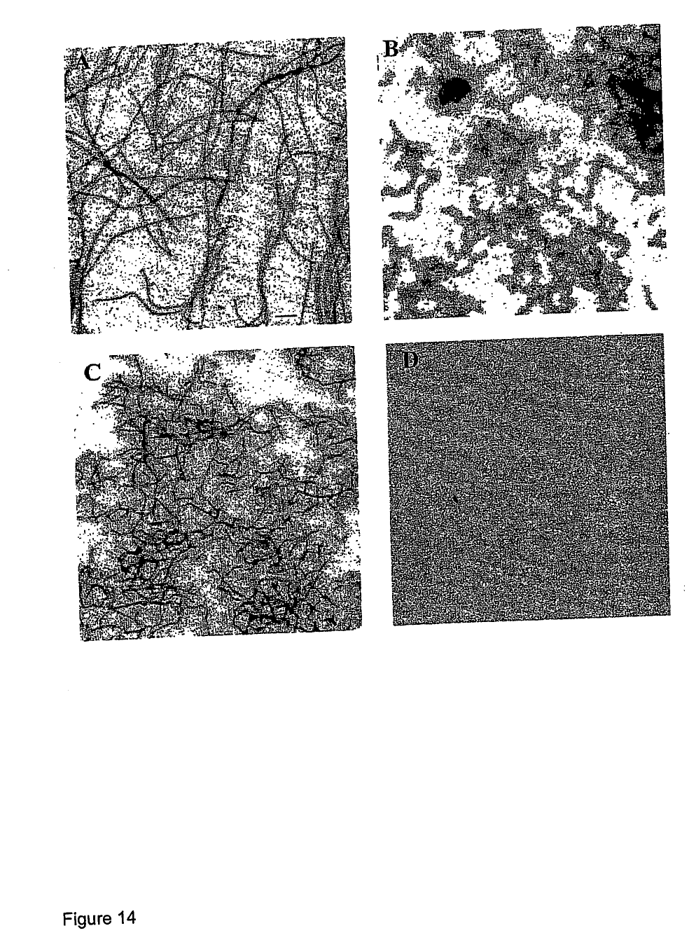

Studies have shown that nucleocapsid (NC) protein binds preferentially to the alternating base sequence d(TG)ₙ in vitro(Fisher et al., 1998). To test if d(TG)ₙ also promotes CA-NC assembly in vitro, assembly reactions were performed by incubating the CA-NC(G94D) protein with four different oligonucleotides: 1) d(TG)₂₅, a 50-base oligonucleotide with 25 repeats of alternating TG sequence; 2) d(TG)₃₈, a 76-base oligonucleotide with 38 repeats of alternating TG sequence; 3) d(TG)₅₀, a 100-base oligonucleotide with 50 repeats of alternating TG sequence; 4) d(N)₁₀₀, a random 100-base oligonucleotide (5′ GCAGTCGAGGAGCAGTCCTCAGTTTGCTTGGGTTACATTAGCCCTTGCTA GTGCTTGAAGGAGTATCGAAACGGAGGTAACCTGTTCGCTGTCCCAGGT G 3′ SEQ ID NO:8). The reactions were carried overnight at 4° C. under the following conditions: 0.3 mg/ml (9 μM) CA-NC(G94D), 0.03 mg/ml (1 μM) of oligonucleotide (approximately 11 nt/l protein molecule), 500 mM NaCl, 50 mM Tris-HCl (pH 8.0). Cylinder formation was measured by light scattering at 312 nm (Abs₃₁₂) (Table 4) and by negatively stained EM (FIG. 14).

TABLE 4
CA-NC (G94D) assembly with different oligonucleotides.

| Oligonucleotide | Abs₃₁₂ |
|---|---|
| d(TG)₂₅ | 0.200 ± 0.021 |
| d(TG)₃₈ | 0.279 ± 0.032 |
| d(TG)₅₀ | 0.292 ± 0.029 |
| d(N)₁₀₀ | 0.035 ± 0.008 |

Both assays reveal that CA-NC assembly is promoted by alternating TG repeats. With a random sequence of oligonucleotide, the assembly detected by light scattering was only slightly above background levels. In contrast, significant light scattering was observed for DNA templates containing alternating repeats of TG oligonucleotides (Abs₃₁₂>0.2). CA-NC assembly is also generally promoted by longer oligonucleotides. The Abs₃₁₂ rose from 0.200 to 0.292 when the length of oligonucleotides increased from 50 to 100. The light scattering data results were also confirmed by TEM in negatively stained samples (FIG. 14).

### 7. Example 7

### Mutations in CA Disrupt the CA-NC Assembly

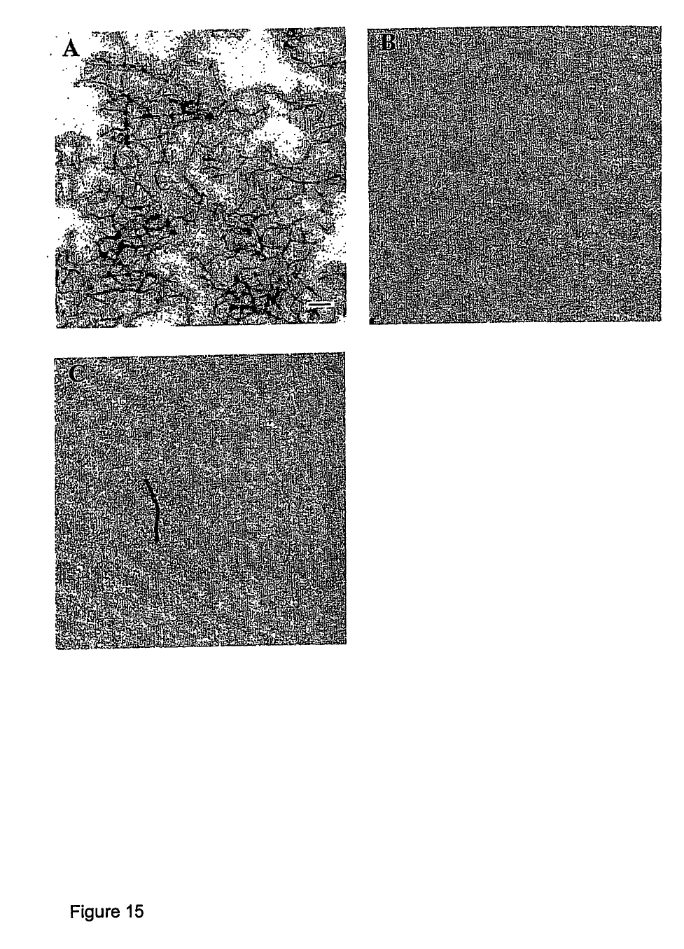

To determine whether the CA-NC cylinders assembled in vitro mimic the mature viral cores formed in vivo, surface point mutations that blocked viral cone formation and replication in vivo were introduced into CA-NC (von Schwedler et al., 1997; EMBO J., 17(6):1555-15, Gamble et al, 1997). The mutations tested were: 1) CA A42D, located in helix 2 of N-terminal of CA. This point mutation blocked cone formation in vivo and rendered the virions noninfectious. 2) CA W184A/M185A, located in the dimer interface of C-terminal of CA. This double point mutant abolished CA dimerization in vitro and blocked capsid assembly and viral replication in vivo. The two different mutations were introduced into the CA-NC (G94D) construct (WISP9868) using single-stranded mutagenesis (Kunkel et al., 1987). The resulting plasmids were named WISP01125 (A42D) and WISP01127 (W184A/M185A). The mutant recombinant proteins were expressed and purified as described previously. Assembly reactions were performed by incubating the mutant proteins with d(TG)₅₀ overnight at 4° C. under the following conditions: 0.3 mg/ml (9 μM) of mutant protein, 0.03 mg/ml (1 μM) of d(TG)₅₀ (approximately 11 nt/l protein molecule), 500 mM NaCl, 50 mM Tris-HCl (pH 8.0). Cylinder formation was measured by light scattering at 312 nm (Abs₃₁₂) (Table 5) and by negatively stained EM (FIG. 15).

TABLE 5
CA-NC(G94D) mutants assembly with d(TG)₅₀.

| Mutations in CA-NC(G94D) | Abs₃₁₂ |
|---|---|
| None | 0.292 ± 0.029 |
| A42D | 0.025 ± 0.013 |
| W184A/M185A | 0.008 ± 0.001 |

Both mutations in N-terminal (A42D) and C-terminal (W184A/M185A) domains of CA abolished CA-NC assembly. It was thus concluded that the sequence requirements for HIV-1 capsid assembly and CA-NC/DNA assembly in vitro are similar.

1. A composition for assaying conformational change of a CA protein comprising a CA protein which has a modification forming a modified CA protein, wherein the modified CA protein comprises a ˜600 Å³ cavity.

2. The composition of claim 1, wherein the composition comprises an N-terminal domain of CA.

3. The composition of claim 2, wherein the N-terminal domain comprises seven alpha helices.

4. The composition of 2, wherein the N-terminal domain comprises amino acids 1-142, 1-143, 1-144, 1-145, 1-146, 1-147, 1-148, 1-149, 1-150, 1-151, 1-152, 1-153, 1-154, 1-155, or 1-156 of SEQ ID NO:1:

5. The composition of claim 2, wherein the N-terminal domain comprises a Proline at the N-terminus.

6. The composition of claim 1, wherein the modified CA protein comprises a helix 6, and wherein the helix 6 is exposed more in the immature structure of the modified CA protein than in the mature structure of the modified CA protein.

7. The composition of claim 1, wherein the modification occurs in helix 1, 3, 6, or the β-hairpin of the CA protein.

8. The composition of claim 7, wherein the modification can react with a chemical reagent.

9. The composition of claim 8, wherein the chemical reagent comprises a thiol.

10. The composition of claim 8, wherein the modification is a cysteine or methioneine substitution.

11. The composition of claim 10, wherein the cysteine substitution occurs at the Ile at position 115 of SEQ ID NO: 1.

12. The composition of claim 6, wherein helix 6 is defined by residues 110 to about 123 of SEQ ID NO: 1.

13. The composition of claim 6, wherein helix 6 is defined by residues 112 to about 120 of SEQ ID NO: 1.

14. The composition of claim 1, wherein the modification occurs at a residue which is more exposed in the immature conformation of the CA protein than in the mature conformation of the CA protein.

15. The composition of claim 1, wherein the modified CA protein is modified by having a molecule attached to the CA protein.

16. The composition of claim 15, wherein the molecule is attached in the region of helix 1, 3, 6, or the β-hairpin.

17. The composition of claim 15, wherein the molecule is a ligand for an antibody.

18. The composition of claim 15, wherein the molecule is biotin.

19. The composition of claim 15, wherein the molecule is digoxygenin.

20. The composition of claim 1, wherein the modified CA protein comprises SEQ ID NO: 11.

21. The composition of claim 1, wherein the composition further comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid(s) added to the N terminus of the CA protein.

22. The composition of claim 21, wherein there are four amino acids added.

23. The composition of claim 22, wherein the modified CA protein has the sequence set forth in SEQ ID NO:3.

24. The composition of claim 21, wherein there are 29 amino acids added.

25. The composition of claim 24, wherein the modified CA protein has the sequence set forth in SEQ ID NO:5.

26. The composition of claim 1, wherein the modified CA protein comprises a histidine tag.

27. The composition of claim 26, wherein the histidine tag comprises 6 histidine residues.

28. A composition comprising a modified CA protein, wherein the modified CA protein can be used to determine whether the ˜600 Å³ cavity of the modified CA protein is accessible.

29. A method determining whether a molecule inhibits the mature conformation of a CA protein comprising incubating the molecule with a CA protein forming a molecule-CA protein mixture and assaying whether the molecule inhibits the hairpin up conformation of the CA protein.

30. A method determining whether a molecule inhibits the mature conformation of a CA protein comprising incubating the molecule with a modified CA protein forming a molecule-CA modified CA protein mixture and assaying whether the molecule inhibits the hairpin up conformation of the modified CA protein.

31. The method of claim 30, wherein the modified CA protein comprises the modified CA protein claim 1.

32. A method of screening for molecules that inhibit maturation of HIV-1 CA protein comprising interacting a target molecule with a modified HIV-1 CA protein, forming a molecule-HIV-1 CA protein mixture and collecting the molecules that reduce the occupation of the ˜600 Å³ cavity of the modified CA protein.

33. A method of testing a molecule for inhibition of maturation of CA protein comprising (a) interacting a target molecule with a CA protein, forming a molecule-CA protein mixture, (b) determining whether the molecule stabilizes the immature conformation of the CA protein.

34. A method of testing a molecule for inhibition of maturation of CA protein comprising (a) interacting a target molecule with a modified CA protein, thereby forming a molecule-modified CA protein mixture, (b) determining whether the molecule stabilizes the immature conformation of the modified CA protein.

35. The method of claim 34, wherein the modified CA protein is the modified CA protein claim 1.

36. The method of claim 35, wherein the modified CA protein is the modified CA protein of claim 10.

37. The method of claim 36, wherein the step of determining the reactivity of the cysteine or methioneine is reactive with a reagent comprising a thiol.

38. The method claim 34, further comprising the step of repeating steps a) and b) with a set of molecules.

39. The method of claim 38 further comprising the step of selecting the molecules which stabilize the immature conformation of the modified CA protein.

40. A method of testing a molecule for the potential to inhibit HIV-1 capsid maturation comprising incubating the molecule with a modified HIV-1 CA protein comprising a ˜600 Å³ cavity forming a molecule-modified CA protein mixture, and determining whether the molecule binds the ˜600 Å³ cavity of the modified CA protein.

41. A method for testing a molecule for the potential to inhibit HIV-1 capsid maturation comprising incubating the molecule with a modified HIV-1 CA protein forming a modified HIV-1 CA protein mixture, and determining whether the molecule inhibits ˜600 Å³ cavity occupation in vitro.

42. The method of claim 29, wherein the mixture further comprises a salt.

43. The method of claim 42, wherein the salt content is less than 2M, 1.5M, IM, 0.9M, 0.8M, 0.7M, 0.6M, 0.5M, 0.4M, 0.3M, 0.2M, 0.1M, 0.05M, or 0.02M.

44. The method of claim 42, wherein the salt content is 500 mM or 150 mM.

45. The method of claim 42, wherein the salt is a monovalent, divalent, or trivalent salt.

46. The method of claim 45, wherein the salt is Mg⁺² Mn⁺² Na⁺, or K⁺.

47. The method of claim 29, wherein the mixture is at a pH between 5 and 10.

48. The method of claim 47, wherein the pH is between 6 and 9.

49. The method of claim 47, wherein the pH is between 6 and 8.

50. The method of claim 29, wherein the mixture is a pH of about 7.2.

51. The method of any of claim 29, wherein the incubation is performed at a temperature of 4-40° C.

52. The method of claim 51, wherein the incubation is performed at 35° C., 30° C., 25° C., 20° C., 15° C., 10° C., 9° C., 8° C., 7° C., 6° C., 5° C. or 4° C.

53. The method of claim 52, wherein the incubation is performed at 4° C.

54. The method of claim 29, wherein the step of determining comprises monitoring a chemical reaction that occurs in the CA protein.

55. The method of claim 29, wherein the step of determining includes a chemical or enzymatic manipulation of the CA protein.

56. The method of claim 55, wherein the step of determining includes assaying radioactivity or fluorescence.

57. The method of claim 29, wherein the step of determining includes assaying radioactivity or fluorescence.

58. A composition comprising a molecule isolated from the method from the method of claim 29.

59. The composition of claim 57, wherein the molecule interacts with Prol or Asp 50 SEQ ID NO:1, wherein the interaction reduces a salt bridge between Pro1 and Asp 50.

60. A composition comprising a modified CA carboxy terminal domain dimer, wherein the dimer is more stable than the dimer naturally.

61. A composition comprising a modified CA carboxy terminal domain dimer, wherein the K_{d} of formation of the modified dimer is less than the K_{d} of formation of a non-modified dimer of the CA carboxy terminal domain.

62. The composition of claim 60, wherein the dimer comprises a sequence having 90% identity to the sequence set forth in SEQ ID NO: 11, or a conserved variant or fragment thereof.

63. The composition of claim 60, wherein the dimer comprises amino acids having 80% identity to amino acids 140-231, 141-231, 142-231, 143-231, 144-231, 145-231, 146-231, 147-231, 148-231 149-231, 150-231, 151-231 set forth in SEQ ID NO: 1, or a conserved variant or fragment thereof.

64. The composition of claim 60, wherein the dimer comprises two carboxy terminal domains.

65. The composition of claim 64, wherein the CA carboxy terminal domains are covalently linked.

66. The composition of claim 64, wherein the CA carboxy terminal domains are covalently linked by amino acids.

67. The composition claim 60, wherein the dimer or one of the CA carboxy terminal domains further comprises the amino acid sequence set forth in SEQ ID NO: 22.

68. A composition comprising a modified CA carboxy terminal domain dimer, wherein the modified CA carboxy terminal domain dimer comprises a first and a second carboxy terminal domain.

69. The composition of claim 68, wherein the K_{d} of formation of the modified dimer is less than or equal to 40 μM or 20 μM or 10 μM or 5 μM or 2.5 μM or 1 μM or 500 nM or 250 nM or 100 nM or 10 nM or 1 nM or 0.1 nM or 0.01 nM.

70. A composition comprising a dimer of CA proteins wherein the dimer comprises a first and a second carboxy terminal domain, wherein the dimer has a K_{d} of less than or equal to 10 μM or 5 μM or 2.5 μM or 1 μM or 500 nM or 250 nM or 100 nM or 10 nM or 1 nM or 0.1 nM or 0.01 nM.

71. The composition of claim 68, wherein the first and second carboxy terminal domains comprise a sequence having 90% identity to the sequence set forth in SEQ ID NO: 11, or a conserved variant or fragment thereof.

72. The composition of claim 68, wherein the dimer comprises amino acids having 80% identity to amino acids 140-231, 141-231, 142-231, 143-231, 144-231, 145-231, 146-231, 147-231, 148-231 149-231, 150-231, 151-231 set forth in SEQ ID NO: 1, or a conserved variant or fragment thereof

73. The composition of claim 68, wherein the first and second carboxy terminal domains are covalently linked.

74. The composition of claim 68, wherein the first and second carboxy terminal domains are covalently linked by amino acids.

75. The composition of claim 68, wherein one of the CA carboxy terminal domains further comprises the amino acid sequence set forth in SEQ ID NO: 22.

76. A composition comprising a modified dimer of CA comprising a molecule having the structure CA-L-CA.

77. The composition of claim 76, wherein the CA comprises a sequence having 90% identity to the sequence set forth in SEQ ID NO: 11, or a conserved variant or fragment thereof.

78. The composition of claim 76, wherein the dimer comprises amino acids having 80% identity to amino acids 140-231, 141-231, 142-231, 143-231, 144-231, 145-231, 146-231, 147-231, 148-231 149-231, 150-231, 151-231 set forth in SEQ ID NO: 1, or a conserved variant or fragment thereof.

79. The composition of claim 76, wherein the CAs are covalently linked.

80. The composition of claim 76, wherein the CAs are covalently linked by amino acids.

81. The composition of claim 76, further comprising the amino acid sequence set forth in SEQ ID NO: 22.

82. The composition of claim 76, wherein L comprises amino acid(s).

83. The composition of claim 76, wherein L comprises a biotin streptavidin pair.

84. The composition of claim 76, wherein L has a length less than or equal to 360 Å, 300 Å, 250 Å, 200 Å, 150 Å, 100 Å, 75 Å, 50 Å, 36 Å, 30 Å, 25 Å, 20 Å, 15 Å, 10 Å, 9 Å, 8 Å, 7 Å, 6 Å, 5 Å, 4 Å, 3 Å, 2 Å, or 1 Å.

85. The composition of claim 76, wherein L comprises 25, 24, 23, 22, 21, 20, 19, 18, 17, 16, 15, 14, 13, 12, 11, 10, 9, 8, 7, 6, 5, 4, 3, or 2 amino acids.

86. The composition of claim 85, wherein L comprises the amino acid glycine, proline, or serine.

87. The composition of claim 85, wherein L comprises 2 amino acids.

88. The composition of claim 87, wherein L comprises the amino acid sequence PW.

89. The composition of claim 76, wherein L comprises a polymer.

90. The composition of claim 89, wherein the polymer is polyethylene glycol (PEG), polypropylene glycol (PPG), polysaccharides, polyamides (nylon), polyesters, polycarbonates, polyphosphates, polyvinyl alcohol, polyethylene, polypropylene, polymethacrylic acids, polysiloxanes, or copolymers thereof.

91. The composition of claim 89, wherein the polymer is less than 200 or 150 or 100 or 90 or 80 or 70 or 60 or 40 or 30 or 20 or 10 or 5 units in length.

92. The composition of claim 76, wherein the CA-L-CA further comprises an additional L-CA.

93. The composition of claim 92, wherein the CA-L-CA further comprises an additional L-CA.

94. The composition of claim 93, wherein the CA-L-CA further comprises an additional L-CA.

95. A method of screening for molecules that inhibit CA carboxy terminal domain dimerization comprising interacting a target molecule with a CA carboxy terminal domain forming a molecule-CA carboxy terminal domain mixture and then interacting the mixture with the composition of claim 60.

96. A method of screening for molecules that inhibit carboxy terminal domain dimerization comprising (a) interacting a target molecule with a CA carboxy terminal domain forming a molecule-CA carboxy terminal domain mixture, (b) removing unbound molecules, (c) interacting the mixture with the composition of claim 60, and (d) collecting the molecules that interact with the composition of claim 60 forming a collection of CA carboxy terminal domain molecules.

97. The method of claim 96, further comprising the step of repeating steps a-d with a collection of the molecules.

98. A method of screening for molecules that inhibit CA carboxy terminal domain dimerization comprising forming a dimer of the composition of claim 60 making a dimer solution, interacting a target molecule with the dimer solution, and determining the amount of dimer present in the dimer solution.

99. A method of determining the effect of a compound on cylindrical formation of a CA-NC protein comprising incubating a modified CA-NC protein, an oligonucleotide, and the compound, and assaying the amount of cylindrical formation in the presence of the compound.

100. The method of claim 99, wherein the CA-NC protein comprises a modification, wherein the modification reduces aggregation of the CA-NC protein.

101. The method of claim 99, wherein the modified CA-NC protein comprises a sequence having 80% identity to SEQ ID NO:20, and wherein the modified CA-NC protein has a D at position 94 of SEQ ID NO:20.

102. The method of claim 99, wherein the CA-NC protein comprises a sequence having 90% identity to the sequence set forth in SEQ ID NO: 11, or a conserved variant or fragment thereof.

103. The method of claim 99, wherein the oligonucleotide is less than 15,000, 14,000, 13,000, 12,000, 11,000, 10,000, 9,000, 8,000, 7,000, 6,000, 5,000, 4,000, 3,000, 2000, 1900, 1800, 1700, 1600, 1500, 1400, 1300, 1200, 1100, 1000, 900, 800, 700, 600, 500, 400, 300, 200, or 100 nucleotides long.

104. The method of claim 103, wherein the oligonucleotide comprises a sequence of TGTG or GTGT.

105. The method of claim 104, wherein the oligonucleotide comprises 300, 250, 200, 150, 100, 90, 80, 70, 60, 50, 45, 40, 35, 30, 25, 20, 15, 10, or 5 d(TG) units.

106. The method of claim 105, wherein the oligonucleotide comprises the sequence set forth in SEQ ID NO:28.

107. The method of claim 99, wherein the concentration of the oligonucleotide is 1 μM.

108. The method of claim 99, wherein the step of assaying comprises monitoring light scattering.

109. The method of claim 105, wherein the monitoring occurs at 312 nM.

110. The method of claim 99, wherein the step of incubating occurs at between 4-40° C.

111. The method of claim 110, wherein the step of incubating occurs a 4 degrees C.

112. The method of claim 99, wherein the concentration of CA-NC protein is less than 10 uM.

113. The method of claim 99, wherein the salt content comprises a monovalent, divalent or trivalent salt.

114. The method of claim 1 13, wherein the salt content comprises Mg^{+2,} Mn^{+2,} Na⁺, or K⁺.

115. The method of claim 113, wherein the step of incubating occurs in a mixture having a salt content of less than 2M, 1.5M, 1M, 0.9M, 0.8M, 0.7M, 0.6M, 0.5M, 0.4M, 0.3M, 0.2M, 0.1M, 0.05M, or 0.02M.

116. The method of claim 115, wherein the step of incubating occurs in a mixture having a salt content less than 500 mM or 50 mM.

117. The method of claim 99, wherein the step of incubating is performed at a pH less than 10, 9, 8, 7, 6, or 5.

118. The method of claim 99, wherein the step of incubating is performed at a pH greater than 10, 9, 8, 7, 6, or 5.

119. The method of claim 99, wherein the step of incubating is performed at a pH of 8 or 7.2.

120. A method of screening for a molecule that inhibits of HIV-1 capsid formation comprising incubating a set of molecules with HIV-1 capsid proteins forming a molecule-capsid protein mixture, determining whether the capsid proteins assemble in vitro, and enriching the molecules that inhibit capsid formation.
